(19) United States
(12) Patent Application Publication
(10) Pub. No.: US 2003/0109498 A1
(43) Pub. Date: Jun. 12, 2003

(54) Antiviral agent for drug-resistant virus

(75) Inventors: Satoshi Yuasa, Kawasaki-shi (JP); Naohiro Kamiya, Kawasaki-shi (JP); Masao Omata, Funabashi-shi (JP)

Correspondence Address: GREENBLUM & BERNSTEIN, P.L.C., 1941 ROLAND CLARKE PLACE, RESTON, VA, 20191, US

(73) Assignee: MITSUBISHI PHARMA CORPORATION, Osaka (JP)

(21) Appl. No.: 09/984,214

(22) Filed: Oct. 29, 2001

(30) Foreign Application Priority Data
Jun. 20, 2001 (KR) 10-2001-0035172

(51) Int. Cl. A61K 31/675

(52) U.S. Cl. 514/81

(57) ABSTRACT

2-amino-6-arylthiopurinephosphonate used in the antiviral agent of the present invention has no toxicity such as bone marrow cell growth inhibition or mutagenicity, and has a high antiviral activity, oral absorbency and safety. Furthermore, the compound of the present invention is effective for a mutant virus which is known to exhibit resistance to the conventional antiviral agents. Therefore, the present invention can provide a useful antiviral agent which leads to the establishment of a method for treating hepatitis B, which is a medically important object.

### TECHNICAL FIELD

The present invention relates to a method for treating a YMDD mutant virus disease which shows a drug-resistance against lamivudine ((−)-β-L-2′,3′-dideoxy-3′-thiacytidine, which is referred to as 3TC) which is a conventional antiviral agent. More specifically, the present invention relates to a method for treating a virus disease which comprises administering a phosphonate nucleotide compound having the above-mentioned antiviral activity, or a salt thereof, or a hydrate thereof or a solvate thereof.

### BACKGROUND ART

Among virus diseases, especially human immunodeficiency virus (HIV) infection (i.e., acquired immune deficiency syndrome (AIDS)) and hepatitis B virus (HBV) infection (i.e., hepatitis B) are considered to be a medically crucial problem because of the high absolute number of patients presented in the world. For the purpose of treating these diseases, various agents having an antiviral activity has been actively developed. Among such agents, lamivudine was developed as the first antiviral agent which inhibits the replication of both HIV and HBV, and it has already been approved as an agent for HIV infection in large number of countries. Lamivudine has recently been approved also for HBV infection in some countries, and there are many reports that lamivudine has clinically been used. The greatest problem regarding the treatment of HBV infection with lamivudine discussed in these reports is that an emergence of drug-resistant viruses increases after the long term administration of lamivudine. It is known that these drug-resistant viruses, as is in the case of HIV, have the mutation of methionine residue in an amino acid sequence, tyrosine-methionine-aspartic acid-aspartic acid (YMDD) motif located in an active center of a viral DNA polymerase. In the case of HIV, there has been reported a result of the X-ray crystallographic analysis of a YMDD mutant polymerase (Sarafianos, S. G. et al., Proc. Natl. Acad. Sci., USA 96, 10027-10032 (1999)). This result suggests a possibility that the substitution of methionine by isoleucine or valine interferes the binding of lamivudine triphosphate to an active center and a possibility of cross-resistance to other nucleoside derivatives having β-L-ring occurs. On the other hand, in the case of HBV, the single substitution of leucine by methionine (L528M) on the B domain of HBV polymerase identified by the clinical trial of Famciclovir (2-[2-(2-amino-9H-purin-9-yl)ethyl]-1,3-propanediol diacetate) against HBV infection shows a resistance to Famciclovir (Seigneres, B., et al., J. Infect. Dis., 181, 1221-1233 (2000)). It is suggested, however, that since a YMDD mutant HBV also having a L528M mutation acquires a higher ability to replicate viral DNA than HBV having a single YMDD mutation, and emerges as a cross drug-resistant HBV (Ono, S. K., et al., J. Clin. Invest., 107, 449-455 (2001)). Summarizing several reports, the emergence rate of a YMDD mutant virus in hepatitis B patients is 17 to 46% after the continuous use of lamivudine for 1 year, and 65 to 75% after the use of lamivudine for 3 to 4 years (Ono, S. K., et al., J. Clin. Invest., 107, 449-455 (2001)). The emergence of a drug-resistant mutant virus reduces the efficacy of the agent. As a result, even if the administration of the agent is continued, proliferation of the virus occurs, resulting in the recurrence of hepatitis B. If the administration of the agent is stopped at this moment, a wild type of HBV having a higher replication ability than drug-resistant mutant virus proliferates again, resulting in the fear of the occurrence of fulminate hepatitis in some cases. However, since it is only lamivudine that has been approved as an antiviral agent against HBV, there is a therapeutic contradiction in that the patients is administered with lamivudine, even if lamivudine is ineffective, and this contradiction would cause some hesitation in administrating the agent to a new hepatitis B patient.

The present inventors have already found that some of phosphonate nucloetide compounds show a high oral absorbency (EP632048), and have obtained an antiviral agent having anti-HBV activity without toxicity such as bone marrow cell growth inhibition or mutagenicity by changing the base portion of those compounds to a specific structure (EP785208). These compounds having a several ten-fold activity as compared with lamivudine, have been developed as novel agents for treating hepatitis B.

The present invention is based on a new finding that the phosphonate nucleotides having a specific structure are effective even against a YMDD mutant HBV and can be a potent tool to overcome the above-stated contradiction regarding the treatment with lamivudine.

### DISCLOSURE OF THE INVENTION

The object of the present invention is to provide a method for treating a drug resistant virus disease by using an antiviral agent effective for the wild type viruses as well as for YMDD mutant viruses which are resistant to antiviral agents such as lamivudine.

Through intensive studies directed toward the above object, the present inventors have found that certain 2-amino-6-arylthiopurinephosphonates, which are not concretely disclosed in the above EP632048, have a high antiviral activity, and that these compound have an inhibitory activity against the proliferation of a YMDD mutant virus, thereby completing the present invention.

Thus, the present invention greatly improves the lamivudine-based method for treating hepatitis B, by using a phosphonate nucleotide compound represented by the following formula (I):

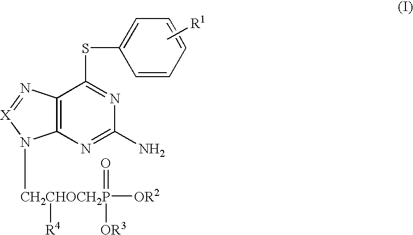

wherein

R¹ is a hydrogen atom, a hydroxyl group, a C₁-C₆ alkoxy group, a C₁-C₄ alkoxy group substituted by one or more halogen atoms, a halogen atom, an amino group or a nitro group;

each of R² and R³ is independently a hydrogen atom, a C₁-C₂₂ alkyl group, an acyloxymethyl group, an acylthioethyl group or an ethyl group substituted by one or more halogen atoms;

R⁴ is a hydrogen atom, a C₁-C₄ alkyl group, a C₁-C₄ hydroxyalkyl group or a C₁-C₄ alkyl group substituted by one ore more halogen atoms; and

X is CH or a nitrogen atom,

or a salt thereof, or a hydrate thereof or a solvate thereof.

### THE BEST MODE FOR CARRYING OUT THE INVENTION

The compounds used in the present invention are described in detail below.

In respect of the phosphonate nucleotide compound of the above formula (I), examples of a C₁-C₆ alkoxy group represented by R¹ include a methoxy group, an ethoxy group, an n-propoxy group, an isopropoxy group, an n-butoxy group, an isobutoxy group, a sec-butoxy group, a tert-butoxy group, an n-pentyloxy group, an n-hexyloxy group and the like. Examples of a C₁-C₄ alkoxy group substituted by one or more halogen atoms represented by R¹ include a monofluoromethoxy group, a difluoromethoxy group, a trifluoromethoxy group, a monofluoroethoxy group, a difluoroethoxy group, a trifluoroethoxy group, a tetrafluoroethoxy group, a pentafluoroethoxy group, a monofluoropropoxy group, a difluoropropoxy group, a trifluoropropoxy group, a tetrafluoropropoxy group, a pentafluoropropoxy group, a hexafluoropropoxy group, a heptafluoropropoxy group, a monofluoroisopropoxy group, a difluoroisopropoxy group, a trifluoroisopropoxy group, a tetrafluoroisopropoxy group, a pentafluoroisopropoxy group, a hexafluoroisopropoxy group, a heptafluoroisopropoxy group and the like.

Examples of a C₁-C₂₂ alkyl group represented by R²and R³ include a methyl group, an ethyl group, an n-propyl group, an isopropyl group, an n-butyl group, an isobutyl group, a sec-butyl group, a tert-butyl group, a pentyl group, a hexyl group, a heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group, an icosyl group, a henicosyl group, a docosyl group and the like.

Examples of an acyloxymethyl group represented by R² and R³ include an acetyloxymethyl group, a propionyloxymethyl group, a butyryloxymethyl group, an isobutyryloxymethyl group, a valeryloxymethyl group, an isovaleryloxymethyl group, a pivaloyloxymethyl group and the like.

Examples of an acylthioethyl group represented by R² and R³ include an acetylthioethyl group, a propionylthioethyl group, a butyrylthioethyl group, an isobutyrylthioethyl group, a valerylthioethyl group, an isovalerylthioethyl group, a pivaloylthioethyl group and the like.

In an ethyl group substituted by one or more halogen atoms represented by R² and R³, the type of the halogen atom may be any of a fluorine, chlorine, bromine or iodine atom. Examples of an ethyl group substituted by one or more halogen atoms include a 1-fluoroethyl group, a 2-fluoroethyl group, a 1-chloroethyl group, a 2-chloroethyl group, a 2-bromoethyl group, a 2,2-difluoroethyl group, a 2,2-dichloroethyl group, a 2,2-dibromoethyl group, a 2,2,2-trifluoroethyl group, a 2,2,2-trichloroethyl group, a 2,2,2-tribromoethyl group and the like. It is particularly preferable that the 2-position of an ethyl group is substituted, and the preferred halogen atom is a fluorine atom. At least one of R² and R³ is preferably an ethyl group substituted by one or more halogen atoms, and is particularly preferably a 2,2,2-trifluoroethyl group.

Examples of a C₁-C₄ alkyl group represented by R⁴ include a methyl group, an ethyl group, an n-propyl group, an isopropyl group, an n-butyl group, an isobutyl group, a sec-butyl group, a tert-butyl group and the like. Examples of a C₁-C₄ hydroxyalkyl group represented by R⁴ include a hydroxymethyl group, a 1-hydroxyethyl group, a 2-hydroxyethyl group, a 1-hydroxypropyl group, a 2-hydroxypropyl group, a 3-hydroxypropyl group, a 1-hydroxybutyl group, a 2-hydroxybutyl group, a 3-hydroxybutyl group, a 4-hydroxybutyl group and the like. Examples of a C₁-C₄ alkyl group substituted by one or more halogen atoms represented by R⁴ include a group in which a halogen atom(s) such as a fluorine atom or a chlorine atom is bound to a methyl group, an ethyl group, an n-propyl group, an isopropyl group, an n-butyl group, an isobutyl group, a sec-butyl group, a tert-butyl group and the like. Specific examples of such a group include a fluoromethyl group, a difluoromethyl group, a trifluoromethyl group, a fluoroethyl group, a chloroethyl group, a fluoropropyl group, a chloropropyl group, a fluorobutyl group, a chlorobutyl group and the like.

The first condition for the preferred compounds used in the present invention is that R¹ is a hydrogen atom, a hydroxyl group or a C₁-C₆ alkoxy group. The second condition for the preferred compound of the present invention is that R¹ is a hydrogen atom, a hydroxyl group or a C₁-C₆ alkoxy group, and each of R² and R³ is independently a hydrogen atom, a C₁-C₂₂ alkyl group or an ethyl group substituted by one or more halogen atoms. Moreover, the third condition for the preferred compound of the present invention is that R¹ is a hydrogen atom, a hydroxyl group or a C₁-C₆ alkoxy group, each of R²and R³ is independently a hydrogen atom, a C₁-C₂₂ alkyl group or a 2,2,2-trifluoroethyl group, and R⁴ is a hydrogen atom or a methyl group. Furthermore, the fourth condition for the preferred compound of the present invention is that R¹ is a hydrogen atom, a hydroxyl group or a C₁-C₆ alkoxy group, each of R² and R³ is independently a hydrogen atom or a 2,2,2-trifluoroethyl group, and R⁴ is a hydrogen atom or a methyl group.

Specific examples of preferred compounds which satisfy this condition include:

2-amino-9-[2-[bis(2,2,2-trifluoroethyl)phosphonylmethoxy]ethyl]-6-phenylthiopurine;

2-amino-9-[2-[bis(2,2,2-trifluoroethyl)phosphonylmethoxy]ethyl]-6-p-methoxyphenylthiopurine;

2-amino-9-[2-[bis(2,2,2-trifluoroethyl)phosphonylmethoxy]ethyl]-6-m-methoxyphenylthiopurine;

2-amino-9-[2-[bis(2,2,2-trifluoroethyl)phosphonylmethoxy]ethyl]-6-o-methoxyphenylthiopurine;

2-amino-9-[2-[bis(2,2,2-trifluoroethyl)phosphonylmethoxy]ethyl]-6-p-ethoxyphenylthiopurine;

2-amino-9-[2-[bis(2,2,2-trifluoroethyl)phosphonylmethoxy]propyl]-6-phenylthiopurine;

2-amino-9-[2-[bis(2,2,2-trifluoroethyl)phosphonylmethoxy]propyl]-6-p-methoxyphenylthiopurine;

2-amino-9-[2-[(2,2,2-trifluoroethyl)phosphonylmethoxy]ethyl]-6-phenylthiopurine;

2-amino-9-[2-[(2,2,2-trifluoroethyl)phosphonylmethoxy]ethyl]-6-p-methoxyphenylthiopurine;

2-amino-9-[2-(phosphonomethoxy)ethyl]-6-phenylthiopurine;

2-amino-9-[2-(phosphonomethoxy)ethyl]-6-p-methoxyphenylthiopurine;

2-amino-9-[2-[bis(2,2,2-trifluoroethyl)phosphonylmethoxy]ethyl]-6-p-butoxyphenylthiopurine;

2-amino-9-[2-[bis(2,2,2-trifluoroethyl)phosphonylmethoxy]ethyl]-6-p-propoxyphenylthiopurine;

2-amino-9-[2-[bis(2,2,2-trifluoroethyl)phosphonylmethoxy]ethyl]-6-p-isopropoxyphenylthiopurine;

2-amino-9-[2-[bis(2,2,2-trifluoroethyl)phosphonylmethoxy]ethyl]-6-p-isobutoxyphenylthiopurine;

2-amino-9-[2-[bis(2,2,2-trifluoroethyl)phosphonylmethoxy]ethyl]-6-p-hydroxyphenylthiopurine;

2-amino-9-[2-[bis(2,2,2-trifluoroethyl)phosphonylmethoxy]ethyl]-6-m-hydroxyphenylthiopurine;

2-amino-9-[2-[bis(2,2,2-trifluoroethyl)phosphonylmethoxy]ethyl]-6-o-hydroxyphenylthiopurine;

2-amino-9-[2-[bis(2,2,2-trifluoroethyl)phosphonylmethoxy]propyl]-6-p-hydroxyphenylthiopurine;

2-amino-9-[2-[bis(2,2,2-trifluoroethyl)phosphonylmethoxy]propyl]-6-m-hydroxyphenylthiopurine; and

2-amino-9-[2-[bis(2,2,2-trifluoroethyl)phosphonylmethoxy]propyl]-6-o-hydroxyphenylthiopurine.

Furthermore, specific examples of preferred compounds which satisfy the fourth condition include:

2-amino-9-[2-[bis(2,2,2-trifluoroethyl)phosphonylmethoxy]ethyl]-6-m-ethoxyphenylthiopurine;

2-amino-9-[2-[bis(2,2,2-trifluoroethyl)phosphonylmethoxy]ethyl]-6-o-ethoxyphenylthiopurine;

2-amino-9-[2-[bis(2,2,2-trifluoroethyl)phosphonylmethoxy]ethyl]-6-m-butoxyphenylthiopurine;

2-amino-9-[2-[bis(2,2,2-trifluoroethyl)phosphonylmethoxy]ethyl]-6-m-propoxyphenylthiopurine;

2-amino-9-[2-[bis(2,2,2-trifluoroethyl)phosphonylmethoxy]ethyl]-6-m-isopropoxyphenylthiopurine;

2-amino-9-[2-[bis(2,2,2-trifluoroethyl)phosphonylmethoxy]ethyl]-6-m-isobutoxyphenylthiopurine;

2-amino-9-[2-[bis(2,2,2-trifluoroethyl)phosphonylmethoxy]ethyl]-6-o-butoxyphenylthiopurine;

2-amino-9-[2-[bis(2,2,2-trifluoroethyl)phosphonylmethoxy]ethyl]-6-o-propoxyphenylthiopurine;

2-amino-9-[2-[bis(2,2,2-trifluoroethyl)phosphonylmethoxy]ethyl]-6-o-isopropoxyphenylthiopurine;

2-amino-9-[2-[bis(2,2,2-trifluoroethyl)phosphonybmethoxy]ethyl]-6-o-isobutoxyphenylthiopurine;

2-amino-9-[2-[bis(2,2,2-trifluoroethyl)phosphonylmethoxy]propyl]-6-m-methoxyphenylthiopurine;

2-amino-9-[2-[bis(2,2,2-trifluoroethyl)phosphonylmethoxy]propyl]-6-o-methoxyphenylthiopurine;

2-amino-9-[2-[bis(2,2,2-trifluoroethyl)phosphonylmethoxy]propyl]-6-p-ethoxyphenylthiopurine;

2-amino-9-[2-[bis(2,2,2-trifluoroethyl)phosphonylmethoxy]propyl]-6-m-ethoxyphenylthiopurine;

2-amino-9-[2-[bis(2,2,2-trifluoroethyl)phosphonylmethoxy]propyl]-6-o-ethoxyphenylthiopurine;

2-amino-9-[2-[bis(2,2,2-trifluoroethyl)phosphonylmethoxy]propyl]-6-p-butoxyphenylthiopurine;

2-amino-9-[2-[bis(2,2,2-trifluoroethyl)phosphonylmethoxy]propyl]-6-p-propoxyphenylthiopurine;

2-amino-9-[2-[bis(2,2,2-trifluoroethyl)phosphonylmethoxy]propyl]-6-p-isopropoxyphenylthiopurine;

2-amino-9-[2-[bis(2,2,2-trifluoroethyl)phosphonylmethoxy]propyl]-6-p-isobutoxyphenylthiopurine;

2-amino-9-[2-[bis(2,2,2-trifluoroethyl)phosphonylmethoxy]propyl]-6-m-butoxyphenylthiopurine;

2-amino-9-[2-[bis(2,2,2-trifluoroethyl)phosphonylmethoxy]propyl]-6-m-propoxyphenylthiopurine;

2-amino-9-[2-[bis(2,2,2-trifluoroethyl)phosphonylmethoxy]propyl]-6-m-isopropoxyphenylthiopurine;

2-amino-9-[2-[bis(2,2,2-trifluoroethyl)phosphonylmethoxy]propyl]-6-m-isobutoxyphenylthiopurine;

2-amino-9-[2-[bis(2,2,2-trifluoroethyl)phosphonylmethoxy]propyl]-6-o-butoxyphenylthiopurine;

2-amino-9-[2-[bis(2,2,2-trifluoroethyl)phosphonylmethoxy]propyl]-6-o-propoxyphenylthiopurine;

2-amino-9-[2-[bis(2,2,2-trifluoroethyl)phosphonylmethoxy]propyl]-6-o-isopropoxyphenylthiopurine;

2-amino-9-[2-[bis(2,2,2-trifluoroethyl)phosphonylmethoxy]propyl]-6-o-isobutoxyphenylthiopurine;

2-amino-9-[2-[(2,2,2-trifluoroethyl)phosphonylmethoxy]ethyl]-6-m-methoxyphenylthiopurine;

2-amino-9-[2-[(2,2,2-trifluoroethyl)phosphonylmethoxy]ethyl]-6-o-methoxyphenylthiopurine;

2-amino-9-[2-[(2,2,2-trifluoroethyl)phosphonylmethoxy]ethyl]-6-p-ethoxyphenylthiopurine;

2-amino-9-[2-[(2,2,2-trifluoroethyl)phosphonylmethoxy]ethyl]-6-m-ethoxyphenylthiopurine;

2-amino-9-[2-[(2,2,2-trifluoroethyl)phosphonylmethoxy]ethyl]-6-o-ethoxyphenylthiopurine;

2-amino-9-[2-[(2,2,2-trifluoroethyl)phosphonylmethoxy]ethyl]-6-p-butoxyphenylthiopurine;

2-amino-9-[2-[(2,2,2-trifluoroethyl)phosphonylmethoxy]ethyl]-6-p-propoxyphenylthiopurine;

2-amino-9-[2-[(2,2,2-trifluoroethyl)phosphonylmethoxy]ethyl]-6-p-isopropoxyphenylthiopurine;

2-amino-9-[2-[(2,2,2-trifluoroethyl)phosphonylmethoxy]ethyl]-6-p-isobutoxyphenylthiopurine;

2-amino-9-[2-[(2,2,2-trifluoroethyl)phosphonylmethoxy]ethyl]-6-m-butoxyphenylthiopurine;

2-amino-9-[2-[(2,2,2-trifluoroethyl)phosphonylmethoxy]ethyl]-6-m-propoxyphenylthiopurine;

2-amino-9-[2-[(2,2,2-trifluoroethyl)phosphonylmethoxy]ethyl]-6-m-isopropoxyphenylthiopurine;

2-amino-9-[2-[(2,2,2-trifluoroethyl)phosphonylmethoxy]ethyl]-6-m-isobutoxyphenylthiopurine;

2-amino-9-[2-[(2,2,2-trifluoroethyl)phosphonylmethoxy]ethyl]-6-o-butoxyphenylthiopurine;

2-amino-9-[2-[(2,2,2-trifluoroethyl)phosphonybmethoxy]ethyl]-6-o-propoxyphenylthiopurine;

2-amino-9-[2-[(2,2,2-trifluoroethyl)phosphonylmethoxy]ethyl]-6-o-isopropoxyphenylthiopurine;

2-amino-9-[2-[(2,2,2-trifluoroethyl)phosphonylmethoxy]ethyl]-6-o-isobutoxyphenylthiopurine;

2-amino-9-[2-[(2,2,2-trifluoroethyl)phosphonylmethoxy]propyl]-6-phenylthiopurine;

2-amino-9-[2-[(2,2,2-trifluoroethyl)phosphonylmethoxy]propyl]-6-p-methoxyphenylthiopurine;

2-amino-9-[2-[(2,2,2-trifluoroethyl)phosphonylmethoxy]propyl]-6-m-methoxyphenylthiopurine;

2-amino-9-[2-[(2,2,2-trifluoroethyl)phosphonylmethoxy]propyl]-6-o-methoxyphenylthiopurine;

2-amino-9-[2-[(2,2,2-trifluoroethyl)phosphonylmethoxy]propyl]-6-p-ethoxyphenylthiopurine;

2-amino-9-[2-[(2,2,2-trifluoroethyl)phosphonylmethoxy]propyl]-6-m-ethoxyphenylthiopurine;

2-amino-9-[2-[(2,2,2-trifluoroethyl)phosphonylmethoxy]propyl]-6-o-ethoxyphenylthiopurine;

2-amino-9-[2-[(2,2,2-trifluoroethyl)phosphonylmethoxy]propyl]-6-p-butoxyphenylthiopurine;

2-amino-9-[2-[(2,2,2-trifluoroethyl)phosphonylmethoxy]propyl]-6-p-propoxyphenylthiopurine;

2-amino-9-[2-[(2,2,2-trifluoroethyl)phosphonylmethoxy]propyl]-6-p-isopropoxyphenylthiopurine;

2-amino-9-[2-[(2,2,2-trifluoroethyl)phosphonylmethoxy]propyl]-6-p-isobutoxyphenylthiopurine;

2-amino-9-[2-[(2,2,2-trifluoroethyl)phosphonylmethoxy]propyl]-6-m-butoxyphenylthiopurine;

2-amino-9-[2-[(2,2,2-trifluoroethyl)phosphonylmethoxy]propyl]-6-m-propoxyphenylthiopurine;

2-amino-9-[2-[(2,2,2-trifluoroethyl)phosphonylmethoxy]propyl]-6-m-isopropoxyphenylthiopurine;

2-amino-9-[2-[(2,2,2-trifluoroethyl)phosphonylmethoxy]propyl]-6-m-isobutoxyphenylthiopurine;

2-amino-9-[2-[(2,2,2-trifluoroethyl)phosphonylmethoxy]propyl]-6-o-butoxyphenylthiopurine;

2-amino-9-[2-[(2,2,2-trifluoroethyl)phosphonylmethoxy]propyl]-6-o-propoxyphenylthiopurine;

2-amino-9-[2-[(2,2,2-trifluoroethyl)phosphonylmethoxy]propyl]-6-o-isopropoxyphenylthiopurine;

2-amino-9-[2-[(2,2,2-trifluoroethyl)phosphonylmethoxy]propyl]-6-o-isobutoxyphenylthiopurine;

2-amino-9-[2-[(2,2,2-trifluoroethyl)phosphonylmethoxy]ethyl]-6-p-hydroxyphenylthiopurine;

2-amino-9-[2-[(2,2,2-trifluoroethyl)phosphonylmethoxy]ethyl]-6-m-hydroxyphenylthiopurine;

2-amino-9-[2-[(2,2,2-trifluoroethyl)phosphonybmethoxy]ethyl]-6-o-hydroxyphenylthiopurine;

2-amino-9-[2-[(2,2,2-trifluoroethyl)phosphonylmethoxy]propyl]-6-p-hydroxyphenylthiopurine;

2-amino-9-[2-[(2,2,2-trifluoroethyl)phosphonylmethoxy]propyl]-6-m-hydroxyphenylthiopurine;

2-amino-9-[2-[(2,2,2-trifluoroethyl)phosphonybmethoxy]propyl]-6-o-hydroxyphenylthiopurine;

2-amino-9-[2-(phosphonomethoxy)ethyl]-6-m-methoxyphenylthiopurine;

2-amino-9-[2-(phosphonomethoxy)ethyl]-6-o-methoxyphenylthiopurine;

2-amino-9-[2-(phosphonomethoxy)ethyl]-6-p-ethoxyphenylthiopurine;

2-amino-9-[2-(phosphonomethoxy)ethyl]-6-m-ethoxyphenylthiopurine;

2-amino-9-[2-(phosphonomethoxy)ethyl]-6-o-ethoxyphenylthiopurine;

2-amino-9-[2-(phosphonomethoxy)ethyl]-6-p-butoxyphenylthiopurine;

2-amino-9-[2-(phosphonomethoxy)ethyl]-6-p-propoxyphenylthiopurine;

2-amino-9-[2-(phosphonomethoxy)ethyl]-6-p-isopropoxyphenylthiopurine;

2-amino-9-[2-(phosphonomethoxy)ethyl]-6-p-isobutoxyphenylthiopurine;

2-amino-9-[2-(phosphonomethoxy)ethyl]-6-m-butoxyphenylthiopurine;

2-amino-9-[2-(phosphonomethoxy)ethyl]-6-m-propoxyphenylthiopurine;

2-amino-9-[2-(phosphonomethoxy)ethyl]-6-m-isopropoxyphenylthiopurine;

2-amino-9-[2-(phosphonomethoxy)ethyl]-6-m-isobutoxyphenylthiopurine;

2-amino-9-[2-(phosphonomethoxy)ethyl]-6-o-butoxyphenylthiopurine;

2-amino-9-[2-(phosphonomethoxy)ethyl]-6-o-propoxyphenylthiopurine;

2-amino-9-[2-(phosphonomethoxy)ethyl]-6-o-isopropoxyphenylthiopurine;

2-amino-9-[2-(phosphonomethoxy)ethyl]-6-o-isobutoxyphenylthiopurine;

2-amino-9-[2-(phosphonomethoxy)propyl]-6-phenylthiopurine;

2-amino-9-[2-(phosphonomethoxy)propyl]-6-p-methoxyphenylthiopurine;

2-amino-9-[2-(phosphonomethoxy)propyl]-6-m-methoxyphenylthiopurine;

2-amino-9-[2-(phosphonomethoxy)propyl]-6-o-methoxyphenylthiopurine;

2-amino-9-[2-(phosphonomethoxy)propyl]-6-p-ethoxyphenylthiopurine;

2-amino-9-[2-(phosphonomethoxy)propyl]-6-m-ethoxyphenylthiopurine;

2-amino-9-[2-(phosphonomethoxy)propyl]-6-o-ethoxyphenylthiopurine;

2-amino-9-[2-(phosphonomethoxy)propyl]-6-p-ethoxyphenylthiopurine;

2-amino-9-[2-(phosphonomethoxy)propyl]-6-p-propoxyphenylthiopurine;

2-amino-9-[2-(phosphonomethoxy)propyl]-6-p-isobutoxyphenylthiopurine;

2-amino-9-[2-(phosphonomethoxy)propyl]-6-p-isobutoxyphenylthiopurine;

2-amino-9-[2-(phosphonomethoxy)propyl]-6-m-propoxyphenylthiopurine;

2-amino-9-[2-(phosphonomethoxy)propyl]-6-m-isobutoxyphenylthiopurine;

2-amino-9-[2-(phosphonomethoxy)propyl]-6-m-isobutoxyphenylthiopurine;

2-amino-9-[2-(phosphonomethoxy)propyl]-6-m-butoxyphenylthiopurine;

2-amino-9-[2-(phosphonomethoxy)propyl]-6-o-propoxyphenylthiopurine;

2-amino-9-[2-(phosphonomethoxy)propyl]-6-o-isopropoxyphenylthiopurine;

2-amino-9-[2-(phosphonomethoxy)propyl]-6-o-isobutoxyphenylthiopurine;

2-amino-9-[2-(phosphonomethoxy)propyl]-6-o-hydroxyphenylthiopurine;

2-amino-9-[2-(phosphonomethoxy)ethyl]-6-p-hydroxyphenylthiopurine;

2-amino-9-[2-(phosphonomethoxy)ethyl]-6-m-hydroxyphenylthiopurine;

2-amino-9-[2-(phosphonomethoxy)ethyl]-6-o-hydroxyphenylthiopurine;

2-amino-9-[2-(phosphonomethoxy)propyl]-6-p-hydroxyphenylthiopurine;

2-amino-9-[2-(phosphonomethoxy)propyl]-6-m-hydroxyphenylthiopurine; and

2-amino-9-[2-(phosphonomethoxy)propyl]-6-o-hydroxyphenylthiopurine.

The fifth condition for the preferred compound of the present invention is that R¹ is a hydrogen atom, a hydroxyl group or a C₁-C₄ alkoxy group, each of R² and R³ is a 2,2,2-trifluoroethyl group, and R⁴ is a hydrogen atom or a methyl group. Specific examples of preferred compounds which satisfy this condition include:

2-amino-9-[2-[bis(2,2,2-trifluoroethyl)phosphonylmethoxy]ethyl]-6-phenylthiopurine;

2-amino-9-[2-[bis(2,2,2-trifluoroethyl)phosphonylmethoxy]ethyl]-6-p-methoxyphenylthiopurine;

2-amino-9-[2-[bis(2,2,2-trifluoroethyl)phosphonylmethoxy]ethyl]-6-m-methoxyphenylthiopurine;

2-amino-9-[2-[bis(2,2,2-trifluoroethyl)phosphonylmethoxy]ethyl]-6-o-methoxyphenylthiopurine;

2-amino-9-[2-[bis(2,2,2-trifluoroethyl)phosphonylmethoxy]ethyl]-6-p-ethoxyphenylthiopurine;

2-amino-9-[2-[bis(2,2,2-trifluoroethyl)phosphonylmethoxy]propyl]-6-phenylthiopurine;

2-amino-9-[2-[bis(2,2,2-trifluoroethyl)phosphonylmethoxy]propyl]-6-p-methoxyphenylthiopurine;

2-amino-9-[2-[bis(2,2,2-trifluoroethyl)phosphonylmethoxy]ethyl]-6-p-butoxyphenylthiopurine;

2-amino-9-[2-[bis(2,2,2-trifluoroethyl)phosphonylmethoxy]ethyl]-6-p-propoxyphenylthiopurine;

2-amino-9-[2-[bis(2,2,2-trifluoroethyl)phosphonylmethoxy]ethyl]-6-p-isopropoxyphenylthiopurine;

2-amino-9-[2-[bis(2,2,2-trifluoroethyl)phosphonylmethoxy]ethyl]-6-p-isobutoxyphenylthiopurine;

2-amino-9-[2-[bis(2,2,2-trifluoroethyl)phosphonylmethoxy]ethyl]-6-p-hydroxyphenylthiopurine;

2-amino-9-[2-[bis(2,2,2-trifluoroethyl)phosphonylmethoxy]ethyl]-6-m-hydroxyphenylthiopurine;

2-amino-9-[2-[bis(2,2,2-trifluoroethyl)phosphonylmethoxy]ethyl]-6-o-hydroxyphenylthiopurine;

2-amino-9-[2-[bis(2,2,2-trifluoroethyl)phosphonylmethoxy]propyl]-6-p-hydroxyphenylthiopurine;

2-amino-9-[2-[bis(2,2,2-trifluoroethyl)phosphonylmethoxy]propyl]-6-m-hydroxyphenylthiopurine; and

2-amino-9-[2-[bis(2,2,2-trifluoroethyl)phosphonylmethoxy]propyl]-6-o-hydroxyphenylthiopurine.

Specific examples of other compounds which satisfy the fifth condition include:

2-amino-9-[2-[bis(2,2,2-trifluoroethyl)phosphonylmethoxy]ethyl]-6-m-ethoxyphenylthiopurine;

2-amino-9-[2-[bis(2,2,2-trifluoroethyl)phosphonylmethoxy]ethyl]-6-o-ethoxyphenylthiopurine;

2-amino-9-[2-[bis(2,2,2-trifluoroethyl)phosphonylmethoxy]ethyl]-6-m-butoxyphenylthiopurine;

2-amino-9-[2-[bis(2,2,2-trifluoroethyl)phosphonylmethoxy]ethyl]-6-m-propoxyphenylthiopurine;

2-amino-9-[2-[bis(2,2,2-trifluoroethyl)phosphonylmethoxy]ethyl]-6-m-isopropoxyphenylthiopurine;

2-amino-9-[2-[bis(2,2,2-trifluoroethyl)phosphonylmethoxy]ethyl]-6-m-isobutoxyphenylthiopurine;

2-amino-9-[2-[bis(2,2,2-trifluoroethyl)phosphonylmethoxy]ethyl]-6-o-butoxyphenylthiopurine;

2-amino-9-[2-[bis(2,2,2-trifluoroethyl)phosphonylmethoxy]ethyl]-6-o-propoxyphenylthiopurine;

2-amino-9-[2-[bis(2,2,2-trifluoroethyl)phosphonylmethoxy]ethyl]-6-o-isopropoxyphenylthiopurine;

2-amino-9-[2-[bis(2,2,2-trifluoroethyl)phosphonylmethoxy]ethyl]-6-o-isobutoxyphenylthiopurine;

2-amino-9-[2-[bis(2,2,2-trifluoroethyl)phosphonylmethoxy]propyl]-6-m-methoxyphenylthiopurine;

2-amino-9-[2-[bis(2,2,2-trifluoroethyl)phosphonylmethoxy]propyl]-6-o-methoxyphenylthiopurine;

2-amino-9-[2-[bis(2,2,2-trifluoroethyl)phosphonylmethoxy]propyl]-6-p-ethoxyphenylthiopurine;

2-amino-9-[2-[bis(2,2,2-trifluoroethyl)phosphonylmethoxy]propyl]-6-m-ethoxyphenylthiopurine;

2-amino-9-[2-[bis(2,2,2-trifluoroethyl)phosphonylmethoxy]propyl]-6-o-ethoxyphenylthiopurine;

2-amino-9-[2-[bis(2,2,2-trifluoroethyl)phosphonylmethoxy]propyl]-6-p-butoxyphenylthiopurine;

2-amino-9-[2-[bis(2,2,2-trifluoroethyl)phosphonylmethoxy]propyl]-6-p-propoxyphenylthiopurine;

2-amino-9-[2-[bis(2,2,2-trifluoroethyl)phosphonylmethoxy]propyl]-6-p-isopropoxyphenylthiopurine;

2-amino-9-[2-[bis(2,2,2-trifluoroethyl)phosphonylmethoxy]propyl]-6-p-isobutoxyphenylthiopurine;

2-amino-9-[2-[bis(2,2,2-trifluoroethyl)phosphonylmethoxy]propyl]-6-m-butoxyphenylthiopurine;

2-amino-9-[2-[bis(2,2,2-trifluoroethyl)phosphonylmethoxy]propyl]-6-m-propoxyphenylthiopurine;

2-amino-9-[2-[bis(2,2,2-trifluoroethyl)phosphonylmethoxy]propyl]-6-m-isopropoxyphenylthiopurine;

2-amino-9-[2-[bis(2,2,2-trifluoroethyl)phosphonylmethoxy]propyl]-6-m-isobutoxyphenylthiopurine;

2-amino-9-[2-[bis(2,2,2-trifluoroethyl)phosphonylmethoxy]propyl]-6-o-butoxyphenylthiopurine;

2-amino-9-[2-[bis(2,2,2-trifluoroethyl)phosphonylmethoxy]propyl]-6-o-propoxyphenylthiopurine;

2-amino-9-[2-[bis(2,2,2-trifluoroethyl)phosphonylmethoxy]propyl]-6-o-isopropoxyphenylthiopurine; and

2-amino-9-[2-[bis(2,2,2-trifluoroethyl)phosphonylmethoxy]propyl]-6-o-isobutoxyphenylthiopurine.

The sixth condition for the preferred compound of the present invention is that R¹ is a hydrogen atom, a hydroxyl group or a C₁-C₄ alkoxy group, each of R² and R³ is a 2,2,2-trifluoroethyl group, and R⁴ is a hydrogen atom. Specific examples of preferred compounds which satisfy this condition include:

2-amino-9-[2-[bis(2,2,2-trifluoroethyl)phosphonylmethoxy]ethyl]-6-phenylthiopurine;

2-amino-9-[2-[bis(2,2,2-trifluoroethyl)phosphonylmethoxy]ethyl]-6-p-methoxyphenylthiopurine;

2-amino-9-[2-[bis(2,2,2-trifluoroethyl)phosphonylmethoxy]ethyl]-6-m-methoxyphenylthiopurine;

2-amino-9-[2-[bis(2,2,2-trifluoroethyl)phosphonylmethoxy]ethyl]-6-o-methoxyphenylthiopurine;

2-amino-9-[2-[bis(2,2,2-trifluoroethyl)phosphonylmethoxy]ethyl]-6-p-ethoxyphenylthiopurine;

2-amino-9-[2-[bis(2,2,2-trifluoroethyl)phosphonylmethoxy]ethyl]-6-p-butoxyphenylthiopurine;

2-amino-9-[2-[bis(2,2,2-trifluoroethyl)phosphonylmethoxy]ethyl]-6-p-propoxyphenylthiopurine;

2-amino-9-[2-[bis(2,2,2-trifluoroethyl)phosphonylmethoxy]ethyl]-6-p-isopropoxyphenylthiopurine;

2-amino-9-[2-[bis(2,2,2-trifluoroethyl)phosphonylmethoxy]ethyl]-6-p-isobutoxyphenylthiopurine;

2-amino-9-[2-[bis(2,2,2-trifluoroethyl)phosphonylmethoxy]ethyl]-6-p-hydroxyphenylthiopurine;

2-amino-9-[2-[bis(2,2,2-trifluoroethyl)phosphonylmethoxy]ethyl]-6-m-hydroxyphenylthiopurine; and

2-amino-9-[2-[bis(2,2,2-trifluoroethyl)phosphonylmethoxy]ethyl]-6-o-hydroxyphenylthiopurine.

Specific examples of other preferred compounds which satisfy the sixth condition include:

2-amino-9-[2-[bis(2,2,2-trifluoroethyl)phosphonylmethoxy]ethyl]-6-m-ethoxyphenylthiopurine;

2-amino-9-[2-[bis(2,2,2-trifluoroethyl)phosphonylmethoxy]ethyl]-6-o-ethoxyphenylthiopurine;

2-amino-9-[2-[bis(2,2,2-trifluoroethyl)phosphonylmethoxy]ethyl]-6-m-butoxyphenylthiopurine;

2-amino-9-[2-[bis(2,2,2-trifluoroethyl)phosphonylmethoxy]ethyl]-6-m-propoxyphenylthiopurine;

2-amino-9-[2-[bis(2,2,2-trifluoroethyl)phosphonylmethoxy]ethyl]-6-m-isopropoxyphenylthiopurine;

2-amino-9-[2-[bis(2,2,2-trifluoroethyl)phosphonylmethoxy]ethyl]-6-m-isobutoxyphenylthiopurine;

2-amino-9-[2-[bis(2,2,2-trifluoroethyl)phosphonylmethoxy]ethyl]-6-o-butoxyphenylthiopurine;

2-amino-9-[2-[bis(2,2,2-trifluoroethyl)phosphonylmethoxy]ethyl]-6-o-propoxyphenylthiopurine;

2-amino-9-[2-[bis(2,2,2-trifluoroethyl)phosphonylmethoxy]ethyl]-6-o-isopropoxyphenylthiopurine; and

2-amino-9-[2-[bis(2,2,2-trifluoroethyl)phosphonylmethoxy]ethyl]-6-o-isobutoxyphenylthiopurine.

The seventh condition for the preferred compound of the present invention is that R¹ is a hydrogen atom, a hydroxyl group or a C₁-C₂ alkoxy group, each of R² and R³ is a 2,2,2-trifluoroethyl group, and R⁴ is a hydrogen atom. Specific examples of preferred compounds which satisfy this condition include:

2-amino-9-[2-[bis(2,2,2-trifluoroethyl)phosphonylmethoxy]ethyl]-6-phenylthiopurine;

2-amino-9-[2-[bis(2,2,2-trifluoroethyl)phosphonylmethoxy]ethyl]-6-p-methoxyphenylthiopurine;

2-amino-9-[2-[bis(2,2,2-trifluoroethyl)phosphonylmethoxy]ethyl]-6-m-methoxyphenylthiopurine;

2-amino-9-[2-[bis(2,2,2-trifluoroethyl)phosphonylmethoxy]ethyl]-6-o-methoxyphenylthiopurine;

2-amino-9-[2-[bis(2,2,2-trifluoroethyl)phosphonylmethoxy]ethyl]-6-p-ethoxyphenylthiopurine;

2-amino-9-[2-[bis(2,2,2-trifluoroethyl)phosphonylmethoxy]ethyl]-6-p-hydroxyphenylthiopurine;

2-amino-9-[2-[bis(2,2,2-trifluoroethyl)phosphonylmethoxy]ethyl]-6-m-hydroxyphenylthiopurine; and

2-amino-9-[2-[bis(2,2,2-trifluoroethyl)phosphonylmethoxy]ethyl]-6-o-hydroxyphenylthiopurine.

The phosphonate nucleotide compounds of the formula (I) used in the present invention may exist as a salt, and any use of the salt formed by the above compound falls within the scope of the present invention. Examples of such a salt include a pharmaceutically acceptable salt. Where an acidic group exists, the acidic group is able to form metal salts such as a lithium salt, a sodium salt, a potassium salt, a magnesium salt and a calcium salt, and ammonium salts such as an ammonium salt, a methyl ammonium salt, a dimethyl ammonium salt, a trimethyl ammonium salt and dicyclohexyl ammonium salt. Where an amino group exists, the amino group is able to form mineral acid salts such as hydrochloride, hydrobromide, sulfate, nitrate, phosphate and metaphosphate, and organic acid salts such as methanesulfonate, benzenesulfonate, para-toluenesulfonate, acetate, propionate, tartrate, fumarate, maleate, malate, oxalate, succinate, citrate, benzoate, mandelate, cinnamate, lactate, besylate, valerate, stearate, oleate, lactobionate, ethylsuccinate, semisuccinate, butyrate, palmitate, carbamate, gluconate, laurate, salicylate, tannate and butylsulfonate.

The phosphonate nucleotide compounds of the formula (I) used in the present invention and a salt thereof may exist in the form of a hydrate or a solvate. Any hydrate or solvate formed by the phosphonate nucleotide compounds of the formula (I) used in the present invention and a salt thereof which include preferred compounds specifically shown above, falls within the scope of the present invention. Examples of a solvent capable of forming the solvate include methanol, ethanol, isopropanol, acetone, ethyl acetate, methylene chloride, diisopropyl ether and the like.

Specific examples of compounds used in the present invention are shown in Table 1-a, b (wherein Me- denotes a methyl group, Et- denotes an ethyl group, n-Pr-denotes an n-propyl group, i-Pr- denotes an isopropyl group, n-Bu denotes an n-butyl group, i-Bu denotes an isobutyl group, and t-Bu- denotes a tert-butyl group).

TABLE 1-a

| No. | R¹ | R² | R³ | R⁴ | X |
|---|---|---|---|---|---|
| 1 | H | CF₃CH₂— | CF₃CH₂— | H | CH |
| 2 | o-OCH₃ | CF₃CH₂— | CF₃CH₂— | H | CH |
| 3 | m-OCH₃ | CF₃CH₂— | CF₃CH₂— | H | CH |
| 4 | p-OCH₃ | CF₃CH₂— | CF₃CH₂— | H | CH |
| 5 | o-Cl | CF₃CH₂— | CF₃CH₂— | H | CH |
| 6 | m-Cl | CF₃CH₂— | CF₃CH₂— | H | CH |
| 7 | p-Cl | CF₃CH₂— | CF₃CH₂— | H | CH |
| 8 | o-Br | CF₃CH₂— | CF₃CH₂— | H | CH |
| 9 | m-Br | CF₃CH₂— | CF₃CH₂— | H | CH |
| 10 | p-Br | CF₃CH₂— | CF₃CH₂— | H | CH |
| 11 | o-NH₂ | CF₃CH₂— | CF₃CH₂— | H | CH |
| 12 | m-NH₂ | CF₃CH₂— | CF₃CH₂— | H | CH |
| 13 | p-NH₂ | CF₃CH₂— | CF₃CH₂— | H | CH |
| 14 | o-NO₂ | CF₃CH₂— | CF₃CH₂— | H | CH |
| 15 | m-NO₂ | CF₃CH₂— | CF₃CH₂— | H | CH |
| 16 | p-NO₂ | CF₃CH₂— | CF₃CH₂— | H | CH |
| 17 | o-OC₂H₅ | CF₃CH₂— | CF₃CH₂— | H | CH |
| 18 | m-OC₂H₅ | CF₃CH₂— | CF₃CH₂— | H | CH |
| 19 | p-OC₂H₅ | CF₃CH₂— | CF₃CH₂— | H | CH |
| 20 | p-I | CF₃CH₂— | CF₃CH₂— | H | CH |
| 21 | o-O-n-Pr | CF₃CH₂— | CF₃CH₂— | H | CH |
| 22 | m-O-n-Pr | CF₃CH₂— | CF₃CH₂— | H | CH |
| 23 | p-O-n-Pr | CF₃CH₂— | CF₃CH₂— | H | CH |
| 24 | o-O-i-Pr | CF₃CH₂— | CF₃CH₂— | H | CH |
| 25 | m-O-i-Pr | CF₃CH₂— | CF₃CH₂— | H | CH |
| 26 | p-O-i-Pr | CF₃CH₂— | CF₃CH₂— | H | CH |
| 27 | o-O-n-Bu | CF₃CH₂— | CF₃CH₂— | H | CH |
| 28 | m-O-n-Bu | CF₃CH₂— | CF₃CH₂— | H | CH |
| 29 | p-O-n-Bu | CF₃CH₂— | CF₃CH₂— | H | CH |
| 30 | o-O-i-Bu | CF₃CH₂— | CF₃CH₂— | H | CH |
| 31 | m-O-i-Bu | CF₃CH₂— | CF₃CH₂— | H | CH |
| 32 | p-O-i-Bu | CF₃CH₂— | CF₃CH₂— | H | CH |
| 33 | o-OCF₃ | CF₃CH₂— | CF₃CH₂— | H | CH |
| 34 | m-OCF₃ | CF₃CH₂— | CF₃CH₂— | H | CH |
| 35 | p-OCF₃ | CF₃CH₂— | CF₃CH₂— | H | CH |
| 36 | H | CF₃CH₂— | CF₃CH₂— | H | N |
| 37 | o-OCH₃ | CF₃CH₂— | CF₃CH₂— | H | N |
| 38 | m-OCH₃ | CF₃CH₂— | CF₃CH₂— | H | N |
| 39 | p-OCH₃ | CF₃CH₂— | CF₃CH₂— | H | N |
| 40 | o-Cl | CF₃CH₂— | CF₃CH₂— | H | N |
| 41 | m-Cl | CF₃CH₂— | CF₃CH₂— | H | N |
| 42 | p-Cl | CF₃CH₂— | CF₃CH₂— | H | N |
| 43 | o-Br | CF₃CH₂— | CF₃CH₂— | H | N |
| 44 | m-Br | CF₃CH₂— | CF₃CH₂— | H | N |
| 45 | p-Br | CF₃CH₂— | CF₃CH₂— | H | N |
| 46 | o-NH₂ | CF₃CH₃- | CF₃CH₂— | H | N |
| 47 | m-NH₂ | CF₃CH₂— | CF₃CH₂— | H | N |
| 48 | p-NH₂ | CF₃CH₂— | CF₃CH₂— | H | N |
| 49 | o-NO₂ | CF₃CH₂— | CF₃CH₂— | H | N |
| 50 | m-NO₂ | CF₃CH₂— | CF₃CH₂— | H | N |
| 51 | p-NO₂ | CF₃CH₂— | CF₃CH₂— | H | N |
| 52 | o-OC₂H₅ | CF₃CH₂— | CF₃CH₂— | H | N |
| 53 | m-OC₂H₅ | CF₃CH₂— | CF₃CH₂— | H | N |
| 54 | p-OC₂H₅ | CF₃CH₂— | CF₃CH₂— | H | N |
| 55 | p-I | CF₃CH₂— | CF₃CH₂— | H | N |
| 56 | o-O-n-Pr | CF₃CH₂— | CF₃CH₂— | H | N |
| 57 | m-O-n-Pr | CF₃CH₂— | CF₃CH₂— | H | N |
| 58 | p-O-n-Pr | CF₃CH₂— | CF₃CH₂— | H | N |
| 59 | o-O-i-Pr | CF₃CH₂— | CF₃CH₂— | H | N |
| 60 | m-O-i-Pr | CF₃CH₂— | CF₃CH₂— | H | N |
| 61 | p-O-i-Pr | CF₃CH₂— | CF₃CH₂— | H | N |
| 62 | o-O-n-Bu | CF₃CH₂— | CF₃CH₂— | H | N |
| 63 | m-O-n-Bu | CF₃CH₂— | CF₃CH₂— | H | N |
| 64 | p-O-n-Bu | CF₃CH₂— | CF₃CH₂— | H | N |
| 65 | o-O-i-Bu | CF₃CH₂— | CF₃CH₂— | H | N |
| 66 | m-O-i-Bu | CF₃CH₂— | CF₃CH₂— | H | N |
| 67 | p-O-i-Bu | CF₃CH₂— | CF₃CH₂— | H | N |
| 68 | o-OCF₃ | CF₃CH₂— | CF₃CH₂— | H | N |
| 69 | m-OCF₃ | CF₃CH₂— | CF₃CH₂— | H | N |
| 70 | p-OCF₃ | CF₃CH₂— | CF₃CH₂— | H | N |
| 71 | H | CF₃CH₂— | Me— | H | CH |
| 72 | o-OCH₃ | CF₃CH₂— | Me— | H | CH |
| 73 | m-OCH₃ | CF₃CH₂— | Me— | H | CH |
| 74 | p-OCH₃ | CF₃CH₂— | Me— | H | CH |
| 75 | o-Cl | CF₃CH₂— | Me— | H | CH |
| 76 | m-Cl | CF₃CH₂— | Me— | H | CH |
| 77 | p-Cl | CF₃CH₂— | Me— | H | CH |
| 78 | o-Br | CF₃CH₂— | Me— | H | CH |
| 79 | m-Br | CF₃CH₂— | Me— | H | CH |
| 80 | p-Br | CF₃CH₂— | Me— | H | CH |
| 81 | o-NH₂ | CF₃CH₂— | Me— | H | CH |
| 82 | m-NH₂ | CF₃CH₂— | Me— | H | CH |
| 83 | p-NH₂ | CF₃CH₂— | Me— | H | CH |
| 84 | o-NO₂ | CF₃CH₂— | Me— | H | CH |
| 85 | m-NO₂ | CF₃CH₂— | Me— | H | CH |
| 86 | p-NO₂ | CF₃CH₂— | Me— | H | CH |
| 87 | o-OC₂H₅ | CF₃CH₂— | Me— | H | CH |
| 88 | m-OC₂H₅ | CF₃CH₂— | Me— | H | CH |
| 89 | p-OC₂H₅ | CF₃CH₂— | Me— | H | CH |
| 90 | p-I | CF₃CH₂— | Me— | H | CH |
| 91 | o-O-n-Pr | CF₃CH₂— | Me— | H | CH |
| 92 | m-O-n-Pr | CF₃CH₂— | Me— | H | CH |
| 93 | p-O-n-Pr | CF₃CH₂— | Me— | H | CH |
| 94 | o-O-i-Pr | CF₃CH₂— | Me— | H | CH |
| 95 | m-O-i-Pr | CF₃CH₂— | Me— | H | CH |
| 96 | p-O-i-Pr | CF₃CH₂— | Me— | H | CH |
| 97 | o-O-n-Bu | CF₃CH₂— | Me— | H | CH |
| 98 | m-O-n-Bu | CF₃CH₂— | Me— | H | CH |
| 99 | p-O-n-Bu | CF₃CH₂— | Me— | H | CH |
| 100 | o-O-i-Bu | CF₃CH₂— | Me— | H | CH |
| 101 | m-O-i-Bu | CF₃CH₂— | Me— | H | CH |
| 102 | p-O-i-Bu | CF₃CH₂— | Me— | H | CH |
| 103 | o-OCF₃ | CF₃CH₂— | Me— | H | CH |
| 104 | m-OCF₃ | CF₃CH₂— | Me— | H | CH |
| 105 | p-OCF₃ | CF₃CH₂— | Me— | H | CH |
| 106 | H | CF₃CH₂— | Me— | H | N |
| 107 | o-OCH₃ | CF₃CH₂— | Me— | H | N |
| 108 | m-OCH₃ | CF₃CH₂— | Me— | H | N |
| 109 | p-OCH₃ | CF₃CH₂— | Me— | H | N |
| 110 | o-Cl | CF₃CH₂— | Me— | H | N |
| 111 | m-Cl | CF₃CH₂— | Me— | H | N |
| 112 | p-Cl | CF₃CH₂— | Me— | H | N |
| 113 | o-Br | CF₃CH₂— | Me— | H | N |
| 114 | m-Br | CF₃CH₂— | Me— | H | N |
| 115 | p-Br | CF₃CH₂— | Me— | H | N |
| 116 | o-NH₂ | CF₃CH₂— | Me— | H | N |
| 117 | m-NH₂ | CF₃CH₂— | Me— | H | N |
| 118 | p-NH₂ | CF₃CH₂— | Me— | H | N |
| 119 | o-NO₂ | CF₃CH₂— | Me— | H | N |
| 120 | m-NO₂ | CF₃CH₂— | Me— | H | N |
| 121 | p-NO₂ | CF₃CH₂— | Me— | H | N |
| 122 | o-OC₂H₅ | CF₃CH₂— | Me— | H | N |
| 123 | m-OC₂H₅ | CF₃CH₂— | Me— | H | N |
| 124 | p-OC₂H₅ | CF₃CH₂— | Me— | H | N |
| 125 | p-I | CF₃CH₂— | Me— | H | N |
| 126 | o-O-n-Pr | CF₃CH₂— | Me— | H | N |
| 127 | m-O-n-Pr | CF₃CH₂— | Me— | H | N |
| 128 | p-O-n-Pr | CF₃CH₂— | Me— | H | N |
| 129 | o-O-i-Pr | CF₃CH₂— | Me— | H | N |
| 130 | m-O-i-Pr | CF₃CH₂— | Me— | H | N |
| 131 | p-O-i-Pr | CF₃CH₂— | Me— | H | N |
| 132 | o-O-n-Bu | CF₃CH₂— | Me— | H | N |
| 133 | m-O-n-Bu | CF₃CH₂— | Me— | H | N |
| 134 | p-O-n-Bu | CF₃CH₂— | Me— | H | N |
| 135 | o-O-i-Bu | CF₃CH₂— | Me— | H | N |
| 136 | m-O-i-Bu | CF₃CH₂— | Me— | H | N |
| 137 | p-O-i-Bu | CF₃CH₂— | Me— | H | N |
| 138 | o-OCF₃ | CF₃CH₂— | Me— | H | N |
| 139 | m-OCF₃ | CF₃CH₂— | Me— | H | N |
| 140 | p-OCF₃ | CF₃CH₂— | Me— | H | N |
| 141 | H | CF₃CH₂— | Et— | H | CH |
| 142 | o-OCH₃ | CF₃CH₂— | Et— | H | CH |
| 143 | m-OCH₃ | CF₃CH₂— | Et— | H | CH |
| 144 | p-OCH₃ | CF₃CH₂— | Et— | H | CH |
| 145 | o-Cl | CF₃CH₂— | Et— | H | CH |
| 146 | m-Cl | CF₃CH₂— | Et— | H | CH |
| 147 | p-Cl | CF₃CH₂— | Et— | H | CH |
| 148 | o-Br | CF₃CH₂— | Et— | H | CH |
| 149 | m-Br | CF₃CH₂— | Et— | H | CH |
| 150 | p-Br | CF₃CH₂— | Et— | H | CH |
| 151 | o-NH₂ | CF₃CH₂— | Et— | H | CH |
| 152 | m-NH₂ | CF₃CH₂— | Et— | H | CH |
| 153 | p-NH₂ | CF₃CH₂— | Et— | H | CH |
| 154 | o-NO₂ | CF₃CH₂— | Et— | H | CH |
| 155 | m-NO₂ | CF₃CH₂— | Et— | H | CH |
| 156 | p-NO₂ | CF₃CH₂— | Et— | H | CH |
| 157 | o-OC₂H₅ | CF₃CH₂— | Et— | H | CH |
| 158 | m-OC₂H₅ | CF₃CH₂— | Et— | H | CH |
| 159 | p-OC₂H₅ | CF₃CH₂— | Et— | H | CH |
| 160 | p-I | CF₃CH₂— | Et— | H | CH |
| 161 | o-O-n-Pr | CF₃CH₂— | Et— | H | CH |
| 162 | m-O-n-Pr | CF₃CH₂— | Et— | H | CH |
| 163 | p-O-n-Pr | CF₃CH₂— | Et— | H | CH |
| 164 | o-O-i-Pr | CF₃CH₂— | Et— | H | CH |
| 165 | m-O-i-Pr | CF₃CH₂— | Et— | H | CH |
| 166 | p-O-i-Pr | CF₃CH₂— | Et— | H | CH |
| 167 | o-O-n-Bu | CF₃CH₂— | Et— | H | CH |
| 168 | m-O-n-Bu | CF₃CH₂— | Et— | H | CH |
| 169 | p-O-n-Bu | CF₃CH₂— | Et— | H | CH |
| 170 | o-O-i-Bu | CF₃CH₂— | Et— | H | CH |
| 171 | m-O-i-Bu | CF₃CH₂— | Et— | H | CH |
| 172 | p-O-i-Bu | CF₃CH₂— | Et— | H | CH |
| 173 | o-OCF₃ | CF₃CH₂— | Et— | H | CH |
| 174 | m-OCF₃ | CF₃CH₂— | Et— | H | CH |
| 175 | p-OCF₃ | CF₃CH₂— | Et— | H | CH |
| 176 | H | CF₃CH₂— | Et— | H | N |
| 177 | o-OCH₃ | CF₃CH₂— | Et— | H | N |
| 178 | m-OCH₃ | CF₃CH₂— | Et— | H | N |
| 179 | p-OCH₃ | CF₃CH₂— | Et— | H | N |
| 180 | o-Cl | CF₃CH₂— | Et— | H | N |
| 181 | m-Cl | CF₃CH₂— | Et— | H | N |
| 182 | p-Cl | CF₃CH₂— | Et— | H | N |
| 183 | o-Br | CF₃CH₂— | Et— | H | N |
| 184 | m-Br | CF₃CH₂— | Et— | H | N |
| 185 | p-Br | CF₃CH₂— | Et— | H | N |
| 186 | o-NH₂ | CF₃CH₂— | Et— | H | N |
| 187 | m-NH₂ | CF₃CH₂— | Et— | H | N |
| 188 | p-NH₂ | CF₃CH₂— | Et— | H | N |
| 189 | o-NO₂ | CF₃CH₂— | Et— | H | N |
| 190 | m-NO₂ | CF₃CH₂— | Et— | H | N |
| 191 | p-NO₂ | CF₃CH₂— | Et— | H | N |
| 192 | o-OC₂H₅ | CF₃CH₂— | Et— | H | N |
| 193 | m-OC₂H₅ | CF₃CH₂— | Et— | H | N |
| 194 | p-OC₂H₅ | CF₃CH₂— | Et— | H | N |
| 195 | p-I | CF₃CH₂— | Et— | H | N |
| 196 | o-O-n-Pr | CF₃CH₂— | Et— | H | N |
| 197 | m-O-n-Pr | CF₃CH₂— | Et— | H | N |
| 198 | p-O-n-Pr | CF₃CH₂— | Et— | H | N |
| 199 | o-O-i-Pr | CF₃CH₂— | Et— | H | N |
| 200 | m-O-i-Pr | CF₃CH₂— | Et— | H | N |
| 201 | p-O-i-Pr | CF₃CH₂— | Et— | H | N |
| 202 | o-O-n-Bu | CF₃CH₂— | Et— | H | N |
| 203 | m-O-n-Bu | CF₃CH₂— | Et— | H | N |
| 204 | p-O-n-Bu | CF₃CH₂— | Et— | H | N |
| 205 | o-O-i-Bu | CF₃CH₂— | Et— | H | N |
| 206 | m-O-i-Bu | CF₃CH₂— | Et— | H | N |
| 207 | p-O-i-Bu | CF₃CH₂— | Et— | H | N |
| 208 | o-OCF₃ | CF₃CH₂— | Et— | H | N |
| 209 | m-OCF₃ | CF₃CH₂— | Et— | H | N |
| 210 | p-OCF₃ | CF₃CH₂— | Et— | H | N |
| 211 | H | CF₃CH₂— | H | H | CH |
| 212 | o-OCH₃ | CF₃CH₂— | H | H | CH |
| 213 | m-OCH₃ | CF₃CH₂— | H | H | CH |
| 214 | p-OCH₃ | CF₃CH₂— | H | H | CH |
| 215 | o-Cl | CF₃CH₂— | H | H | CH |
| 216 | m-Cl | CF₃CH₂— | H | H | CH |
| 217 | p-Cl | CF₃CH₂— | H | H | CH |
| 218 | o-Br | CF₃CH₂— | H | H | CH |
| 219 | m-Br | CF₃CH₂— | H | H | CH |
| 220 | p-Br | CF₃CH₂— | H | H | CH |
| 221 | o-NH₂ | CF₃CH₂— | H | H | CH |
| 222 | m-NH₂ | CF₃CH₂— | H | H | CH |
| 223 | p-NH₂ | CF₃CH₂— | H | H | CH |
| 224 | o-NO₂ | CF₃CH₂— | H | H | CH |
| 225 | m-NO₂ | CF₃CH₂— | H | H | CH |
| 226 | p-NO₂ | CF₃CH₂— | H | H | CH |
| 227 | o-OC₂H₅ | CF₃CH₂— | H | H | CH |
| 228 | m-OC₂H₅ | CF₃CH₂— | H | H | CH |
| 229 | p-OC₂H₅ | CF₃CH₂— | H | H | CH |
| 230 | p-I | CF₃CH₂— | H | H | CH |
| 231 | o-O-n-Pr | CF₃CH₂— | H | H | CH |
| 232 | m-O-n-Pr | CF₃CH₂— | H | H | CH |
| 233 | p-O-n-Pr | CF₃CH₂— | H | H | CH |
| 234 | o-O-i-Pr | CF₃CH₂— | H | H | CH |
| 235 | m-O-i-Pr | CF₃CH₂— | H | H | CH |
| 236 | p-O-i-Pr | CF₃CH₂— | H | H | CH |
| 237 | o-O-n-Bu | CF₃CH₂— | H | H | CH |
| 238 | m-O-n-Bu | CF₃CH₂— | H | H | CH |
| 239 | p-O-n-Bu | CF₃CH₂— | H | H | CH |
| 240 | o-O-i-Bu | CF₃CH₂— | H | H | CH |
| 241 | m-O-i-Bu | CF₃CH₂— | H | H | CH |
| 242 | p-O-i-Bu | CF₃CH₂— | H | H | CH |
| 243 | o-OCF₃ | CF₃CH₂— | H | H | CH |
| 244 | m-OCF₃ | CF₃CH₂— | H | H | CH |
| 245 | p-OCF₃ | CF₃CH₂— | H | H | CH |
| 246 | H | CF₃CH₂— | H | H | N |
| 247 | o-OCH₃ | CF₃CH₂— | H | H | N |
| 248 | m-OCH₃ | CF₃CH₂— | H | H | N |
| 249 | p-OCH₃ | CF₃CH₂— | H | H | N |
| 250 | o-Cl | CF₃CH₂— | H | H | N |
| 251 | m-Cl | CF₃CH₂— | H | H | N |
| 252 | p-Cl | CF₃CH₂— | H | H | N |
| 253 | o-Br | CF₃CH₂— | H | H | N |
| 254 | m-Br | CF₃CH₂— | H | H | N |
| 255 | p-Br | CF₃CH₂— | H | H | N |
| 256 | o-NH₂ | CF₃CH₂— | H | H | N |
| 257 | m-NH₂ | CF₃CH₂— | H | H | N |
| 258 | p-NH₂ | CF₃CH₂— | H | H | N |
| 259 | o-NO₂ | CF₃CH₂— | H | H | N |
| 260 | m-NO₂ | CF₃CH₂— | H | H | N |
| 261 | p-NO₂ | CF₃CH₂— | H | H | N |
| 262 | o-OC₂H₅ | CF₃CH₂— | H | H | N |
| 263 | m-OC₂H₅ | CF₃CH₂— | H | H | N |
| 264 | p-OC₂H₅ | CF₃CH₂— | H | H | N |
| 265 | p-I | CF₃CH₂— | H | H | N |
| 266 | o-O-n-Pr | CF₃CH₂— | H | H | N |
| 267 | m-O-n-Pr | CF₃CH₂— | H | H | N |
| 268 | p-O-n-Pr | CF₃CH₂— | H | H | N |
| 269 | o-O-i-Pr | CF₃CH₂— | H | H | N |
| 270 | m-O-i-Pr | CF₃CH₂— | H | H | N |
| 271 | p-O-i-Pr | CF₃CH₂— | H | H | N |
| 272 | o-O-n-Bu | CF₃CH₂— | H | H | N |
| 273 | m-O-n-Bu | CF₃CH₂— | H | H | N |
| 274 | p-O-n-Bu | CF₃CH₂— | H | H | N |
| 275 | o-O-i-Bu | CF₃CH₂— | H | H | N |
| 276 | m-O-i-Bu | CF₃CH₂— | H | H | N |
| 277 | p-O-i-Bu | CF₃CH₂— | H | H | N |
| 278 | o-OCF₃ | CF₃CH₂— | H | H | N |
| 279 | m-OCF₃ | CF₃CH₂— | H | H | N |
| 280 | p-OCF₃ | CF₃CH₂— | H | H | N |
| 281 | H | H | H | H | CH |
| 282 | o-OCH₃ | H | H | H | CH |
| 283 | m-OCH₃ | H | H | H | CH |
| 284 | p-OCH₃ | H | H | H | CH |
| 285 | o-Cl | H | H | H | CH |
| 286 | m-Cl | H | H | H | CH |
| 287 | p-Cl | H | H | H | CH |
| 288 | o-Br | H | H | H | CH |
| 289 | m-Br | H | H | H | CH |
| 290 | p-Br | H | H | H | CH |
| 291 | o-NH₂ | H | H | H | CH |
| 292 | m-NH₂ | H | H | H | CH |
| 293 | p-NH₂ | H | H | H | CH |
| 294 | o-NO₂ | H | H | H | CH |
| 295 | m-NO₂ | H | H | H | CH |
| 296 | p-NO₂ | H | H | H | CH |
| 297 | o-OC₂H₅ | H | H | H | CH |
| 298 | m-OC₂H₅ | H | H | H | CH |
| 299 | p-OC₂H₅ | H | H | H | CH |
| 300 | p-I | H | H | H | CH |
| 301 | o-O-n-Pr | H | H | H | CH |
| 302 | m-O-n-Pr | H | H | H | CH |
| 303 | p-O-n-Pr | H | H | H | CH |
| 304 | o-O-i-Pr | H | H | H | CH |
| 305 | m-O-i-Pr | H | H | H | CH |
| 306 | p-O-i-Pr | H | H | H | CH |
| 307 | o-O-n-Bu | H | H | H | CH |
| 308 | m-O-n-Bu | H | H | H | CH |
| 309 | p-O-n-Bu | H | H | H | CH |
| 310 | o-O-i-Bu | H | H | H | CH |
| 311 | m-O-i-Bu | H | H | H | CH |
| 312 | p-O-i-Bu | H | H | H | CH |
| 313 | o-OCF₃ | H | H | H | CH |
| 314 | m-OCF₃ | H | H | H | CH |
| 315 | p-OCF₃ | H | H | H | CH |
| 316 | H | H | H | H | N |
| 317 | o-OCH₃ | H | H | H | N |
| 318 | m-OCH₃ | H | H | H | N |
| 319 | p-OCH₃ | H | H | H | N |
| 320 | o-Cl | H | H | H | N |
| 321 | m-Cl | H | H | H | N |
| 322 | p-Cl | H | H | H | N |
| 323 | o-Br | H | H | H | N |
| 324 | m-Br | H | H | H | N |
| 325 | p-Br | H | H | H | N |
| 326 | o-NH₂ | H | H | H | N |
| 327 | m-NH₂ | H | H | H | N |
| 328 | p-NH₂ | H | H | H | N |
| 329 | o-NO₂ | H | H | H | N |
| 330 | m-NO₂ | H | H | H | N |
| 331 | p-NO₂ | H | H | H | N |
| 332 | o-OC₂H₅ | H | H | H | N |
| 333 | m-OC₂H₅ | H | H | H | N |
| 334 | p-OC₂H₅ | H | H | H | N |
| 335 | p-I | H | H | H | N |
| 336 | o-O-n-Pr | H | H | H | N |
| 337 | m-O-n-Pr | H | H | H | N |
| 338 | p-O-n-Pr | H | H | H | N |
| 339 | o-O-i-Pr | H | H | H | N |
| 340 | m-O-i-Pr | H | H | H | N |
| 341 | p-O-i-Pr | H | H | H | N |
| 342 | o-O-n-Bu | H | H | H | N |
| 343 | m-O-n-Bu | H | H | H | N |
| 344 | p-O-n-Bu | H | H | H | N |
| 345 | o-O-i-Bu | H | H | H | N |
| 346 | m-O-i-Bu | H | H | H | N |
| 347 | p-O-i-Bu | H | H | H | N |
| 348 | o-OCF₃ | H | H | H | N |
| 349 | m-OCF₃ | H | H | H | N |
| 350 | p-OCF₃ | H | H | H | N |
| 351 | H | CF₃CH₂— | CF₃CH₂— | Me— | CH |
| 352 | o-OCH₃ | CF₃CH₂— | CF₃CH₂— | Me— | CH |
| 353 | m-OCH₃ | CF₃CH₂— | CF₃CH₂— | Me— | CH |
| 354 | p-OCH₃ | CF₃CH₂— | CF₃CH₂— | Me— | CH |
| 355 | o-Cl | CF₃CH₂— | CF₃CH₂— | Me— | CH |
| 356 | m-Cl | CF₃CH₂— | CF₃CH₂— | Me— | CH |
| 357 | p-Cl | CF₃CH₂— | CF₃CH₂— | Me— | CH |
| 358 | o-Br | CF₃CH₂— | CF₃CH₂— | Me— | CH |
| 359 | m-Br | CF₃CH₂— | CF₃CH₂— | Me— | CH |
| 360 | p-Br | CF₃CH₂— | CF₃CH₂— | Me— | CH |
| 361 | o-NH₂ | CF₃CH₂— | CF₃CH₂— | Me— | CH |
| 362 | m-NH₂ | CF₃CH₂— | CF₃CH₂— | Me— | CH |
| 363 | p-NH₂ | CF₃CH₂— | CF₃CH₂— | Me— | CH |
| 364 | o-NO₂ | CF₃CH₂— | CF₃CH₂— | Me— | CH |
| 365 | m-NO₂ | CF₃CH₂— | CF₃CH₂— | Me— | CH |
| 366 | p-NO₂ | CF₃CH₂— | CF₃CH₂— | Me— | CH |
| 367 | o-OC₂H₅ | CF₃CH₂— | CF₃CH₂— | Me— | CH |
| 368 | m-OC₂H₅ | CF₃CH₂— | CF₃CH₂— | Me— | CH |
| 369 | p-OC₂H₅ | CF₃CH₂— | CF₃CH₂— | Me— | CH |
| 370 | p-I | CF₃CH₂— | CF₃CH₂— | Me— | CH |
| 371 | o-O-n-Pr | CF₃CH₂— | CF₃CH₂— | Me— | CH |
| 372 | m-O-n-Pr | CF₃CH₂— | CF₃CH₂— | Me— | CH |
| 373 | p-O-n-Pr | CF₃CH₂— | CF₃CH₂— | Me— | CH |
| 374 | o-O-i-Pr | CF₃CH₂— | CF₃CH₂— | Me— | CH |
| 375 | m-O-i-Pr | CF₃CH₂— | CF₃CH₂— | Me— | CH |
| 376 | p-O-i-Pr | CF₃CH₂— | CF₃CH₂— | Me— | CH |
| 377 | o-O-n-Bu | CF₃CH₂— | CF₃CH₂— | Me— | CH |
| 378 | m-O-n-Bu | CF₃CH₂— | CF₃CH₂— | Me— | CH |
| 379 | p-O-n-Bu | CF₃CH₂— | CF₃CH₂— | Me— | CH |
| 380 | o-O-i-Bu | CF₃CH₂— | CF₃CH₂— | Me— | CH |
| 381 | m-O-i-Bu | CF₃CH₂— | CF₃CH₂— | Me— | CH |
| 382 | p-O-i-Bu | CF₃CH₂— | CF₃CH₂— | Me— | CH |
| 383 | o-OCF₃ | CF₃CH₂— | CF₃CH₂— | Me— | CH |
| 384 | m-OCF₃ | CF₃CH₂— | CF₃CH₂— | Me— | CH |
| 385 | p-OCF₃ | CF₃CH₂— | CF₃CH₂— | Me— | CH |
| 386 | H | CF₃CH₂— | CF₃CH₂— | Me— | N |
| 387 | o-OCH₃ | CF₃CH₂— | CF₃CH₂— | Me— | N |
| 388 | m-OCH₃ | CF₃CH₂— | CF₃CH₂— | Me— | N |
| 389 | p-OCH₃ | CF₃CH₂— | CF₃CH₂— | Me— | N |
| 390 | o-Cl | CF₃CH₂— | CF₃CH₂— | Me— | N |
| 391 | m-Cl | CF₃CH₂— | CF₃CH₂— | Me— | N |
| 392 | p-Cl | CF₃CH₂— | CF₃CH₂— | Me— | N |
| 393 | o-Br | CF₃CH₂— | CF₃CH₂— | Me— | N |
| 394 | m-Br | CF₃CH₂— | CF₃CH₂— | Me— | N |
| 395 | p-Br | CF₃CH₂— | CF₃CH₂— | Me— | N |
| 396 | o-NH₂ | CF₃CH₂— | CF₃CH₂— | Me— | N |
| 397 | m-NH₂ | CF₃CH₂— | CF₃CH₂— | Me— | N |
| 398 | p-NH₂ | CF₃CH₂— | CF₃CH₂— | Me— | N |
| 399 | o-NO₂ | CF₃CH₂— | CF₃CH₂— | Me— | N |
| 400 | m-NO₂ | CF₃CH₂— | CF₃CH₂— | Me— | N |
| 401 | p-NO₂ | CF₃CH₂— | CF₃CH₂— | Me— | N |
| 402 | o-OC₂H₅ | CF₃CH₂— | CF₃CH₂— | Me— | N |
| 403 | m-OC₂H₅ | CF₃CH₂— | CF₃CH₂— | Me— | N |
| 404 | p-OC₂H₅ | CF₃CH₂— | CF₃CH₂— | Me— | N |
| 405 | p-I | CF₃CH₂— | CF₃CH₂— | Me— | N |
| 406 | o-O-n-Pr | CF₃CH₂— | CF₃CH₂— | Me— | N |
| 407 | m-O-n-Pr | CF₃CH₂— | CF₃CH₂— | Me— | N |
| 408 | p-O-n-Pr | CF₃CH₂— | CF₃CH₂— | Me— | N |
| 409 | o-O-i-Pr | CF₃CH₂— | CF₃CH₂— | Me— | N |
| 410 | m-O-i-Pr | CF₃CH₂— | CF₃CH₂— | Me— | N |
| 411 | p-O-i-Pr | CF₃CH₂— | CF₃CH₂— | Me— | N |
| 412 | o-O-n-Bu | CF₃CH₂— | CF₃CH₂— | Me— | N |
| 413 | m-O-n-Bu | CF₃CH₂— | CF₃CH₂— | Me— | N |
| 414 | p-O-n-Bu | CF₃CH₂— | CF₃CH₂— | Me— | N |
| 415 | o-O-i-Bu | CF₃CH₂— | CF₃CH₂— | Me— | N |
| 416 | m-O-i-Bu | CF₃CH₂— | CF₃CH₂— | Me— | N |
| 417 | p-O-i-Bu | CF₃CH₂— | CF₃CH₂— | Me— | N |
| 418 | o-OCF₃ | CF₃CH₂— | CF₃CH₂— | Me— | N |
| 419 | m-OCF₃ | CF₃CH₂— | CF₃CH₂— | Me— | N |
| 420 | p-OCF₃ | CF₃CH₂— | CF₃CH₂— | Me— | N |
| 421 | H | CF₃CH₂— | Me— | Me— | CH |
| 422 | o-OCH₃ | CF₃CH₂— | Me— | Me— | CH |
| 423 | m-OCH₃ | CF₃CH₂— | Me— | Me— | CH |
| 424 | p-OCH₃ | CF₃CH₂— | Me— | Me— | CH |
| 425 | o-Cl | CF₃CH₂— | Me— | Me— | CH |
| 426 | m-Cl | CF₃CH₂— | Me— | Me— | CH |
| 427 | p-Cl | CF₃CH₂— | Me— | Me— | CH |
| 428 | o-Br | CF₃CH₂— | Me— | Me— | CH |
| 429 | m-Br | CF₃CH₂— | Me— | Me— | CH |
| 430 | p-Br | CF₃CH₂— | Me— | Me— | CH |
| 431 | o-NH₂ | CF₃CH₂— | Me— | Me— | CH |
| 432 | m-NH₂ | CF₃CH₂— | Me— | Me— | CH |
| 433 | p-NH₂ | CF₃CH₂— | Me— | Me— | CH |
| 434 | o-NO₂ | CF₃CH₂— | Me— | Me— | CH |
| 435 | m-NO₂ | CF₃CH₂— | Me— | Me— | CH |
| 436 | p-NO₂ | CF₃CH₂— | Me— | Me— | CH |
| 437 | o-OC₂H₅ | CF₃CH₂— | Me— | Me— | CH |
| 438 | m-OC₂H₅ | CF₃CH₂— | Me— | Me— | CH |
| 439 | p-OC₂H₅ | CF₃CH₂— | Me— | Me— | CH |
| 440 | p-I | CF₃CH₂— | Me— | Me— | CH |
| 441 | o-O-n-Pr | CF₃CH₂— | Me— | Me— | CH |
| 442 | m-O-n-Pr | CF₃CH₂— | Me— | Me— | CH |
| 443 | p-O-n-Pr | CF₃CH₂— | Me— | Me— | CH |
| 444 | o-O-i-Pr | CF₃CH₂— | Me— | Me— | CH |
| 445 | m-O-i-Pr | CF₃CH₂— | Me— | Me— | CH |
| 446 | p-O-i-Pr | CF₃CH₂— | Me— | Me— | CH |
| 447 | o-O-n-Bu | CF₃CH₂— | Me— | Me— | CH |
| 448 | m-O-n-Bu | CF₃CH₂— | Me— | Me— | CH |
| 449 | p-O-n-Bu | CF₃CH₂— | Me— | Me— | CH |
| 450 | o-O-i-Bu | CF₃CH₂— | Me— | Me— | CH |
| 451 | m-O-i-Bu | CF₃CH₂— | Me— | Me— | CH |
| 452 | p-O-i-Bu | CF₃CH₂— | Me— | Me— | CH |
| 453 | o-OCF₃ | CF₃CH₂— | Me— | Me— | CH |
| 454 | m-OCF₃ | CF₃CH₂— | Me— | Me— | CH |
| 455 | p-OCF₃ | CF₃CH₂— | Me— | Me— | CH |
| 456 | H | CF₃CH₂— | Me— | Me— | N |
| 457 | o-OCH₃ | CF₃CH₂— | Me— | Me— | N |
| 458 | m-OCH₃ | CF₃CH₂— | Me— | Me— | N |
| 459 | p-OCH₃ | CF₃CH₂— | Me— | Me— | N |
| 460 | o-Cl | CF₃CH₂— | Me— | Me— | N |
| 461 | m-Cl | CF₃CH₂— | Me— | Me— | N |
| 462 | p-Cl | CF₃CH₂— | Me— | Me— | N |
| 463 | o-Br | CF₃CH₂— | Me— | Me— | N |
| 464 | m-Br | CF₃CH₂— | Me— | Me— | N |
| 465 | p-Br | CF₃CH₂— | Me— | Me— | N |
| 466 | o-NH₂ | CF₃CH₂— | Me— | Me— | N |
| 467 | m-NH₂ | CF₃CH₂— | Me— | Me— | N |
| 468 | p-NH₂ | CF₃CH₂— | Me— | Me— | N |
| 469 | o-NO₂ | CF₃CH₂— | Me— | Me— | N |
| 470 | m-NO₂ | CF₃CH₂— | Me— | Me— | N |
| 471 | p-NO₂ | CF₃CH₂— | Me— | Me— | N |
| 472 | o-OC₂H₅ | CF₃CH₂— | Me— | Me— | N |
| 473 | m-OC₂H₅ | CF₃CH₂— | Me— | Me— | N |
| 474 | p-OC₂H₅ | CF₃CH₂— | Me— | Me— | N |
| 475 | p-I | CF₃CH₂— | Me— | Me— | N |
| 476 | o-O-n-Pr | CF₃CH₂— | Me— | Me— | N |
| 477 | m-O-n-Pr | CF₃CH₂— | Me— | Me— | N |
| 478 | p-O-n-Pr | CF₃CH₂— | Me— | Me— | N |
| 479 | o-O-i-Pr | CF₃CH₂— | Me— | Me— | N |
| 480 | m-O-i-Pr | CF₃CH₂— | Me— | Me— | N |
| 481 | p-O-i-Pr | CF₃CH₂— | Me— | Me— | N |
| 482 | o-O-n-Bu | CF₃CH₂— | Me— | Me— | N |
| 483 | m-O-n-Bu | CF₃CH₂— | Me— | Me— | N |
| 484 | p-O-n-Bu | CF₃CH₂— | Me— | Me— | N |
| 485 | o-O-i-Bu | CF₃CH₂— | Me— | Me— | N |
| 486 | m-O-i-Bu | CF₃CH₂— | Me— | Me— | N |
| 487 | p-O-i-Bu | CF₃CH₂— | Me— | Me— | N |
| 488 | o-OCF₃ | CF₃CH₂— | Me— | Me— | N |
| 489 | m-OCF₃ | CF₃CH₂— | Me— | Me— | N |
| 490 | p-OCF₃ | CF₃CH₂— | Me— | Me— | N |
| 491 | H | CF₃CH₂— | Et— | Me— | CH |
| 492 | o-OCH₃ | CF₃CH₂— | Et— | Me— | CH |
| 493 | m-OCH₃ | CF₃CH₂— | Et— | Me— | CH |
| 494 | p-OCH₃ | CF₃CH₂— | Et— | Me— | CH |
| 495 | o-Cl | CF₃CH₂— | Et— | Me— | CH |
| 496 | m-Cl | CF₃CH₂— | Et— | Me— | CH |
| 497 | p-Cl | CF₃CH₂— | Et— | Me— | CH |
| 498 | o-Br | CF₃CH₂— | Et— | Me— | CH |
| 499 | m-Br | CF₃CH₂— | Et— | Me— | CH |
| 500 | p-Br | CF₃CH₂— | Et— | Me— | CH |
| 501 | o-NH₂ | CF₃CH₂— | Et— | Me— | CH |
| 502 | m-NH₂ | CF₃CH₂— | Et— | Me— | CH |
| 503 | p-NH₂ | CF₃CH₂— | Et— | Me— | CH |
| 504 | o-NO₂ | CF₃CH₂— | Et— | Me— | CH |
| 505 | m-NO₂ | CF₃CH₂— | Et— | Me— | CH |
| 506 | p-NO₂ | CF₃CH₂— | Et— | Me— | CH |
| 507 | o-OC₂H₅ | CF₃CH₂— | Et— | Me— | CH |
| 508 | m-OC₂H₅ | CF₃CH₂— | Et— | Me— | CH |
| 509 | p-OC₂H₅ | CF₃CH₂— | Et— | Me— | CH |
| 510 | p-I | CF₃CH₂— | Et— | Me— | CH |
| 511 | o-O-n-Pr | CF₃CH₂— | Et— | Me— | CH |
| 512 | m-O-n-Pr | CF₃CH₂— | Et— | Me— | CH |
| 513 | p-O-n-Pr | CF₃CH₂— | Et— | Me— | CH |
| 514 | o-O-i-Pr | CF₃CH₂— | Et— | Me— | CH |
| 515 | m-O-i-Pr | CF₃CH₂— | Et— | Me— | CH |
| 516 | p-O-i-Pr | CF₃CH₂— | Et— | Me— | CH |
| 517 | o-O-n-Bu | CF₃CH₂— | Et— | Me— | CH |
| 518 | m-O-n-Bu | CF₃CH₂— | Et— | Me— | CH |
| 519 | p-O-n-Bu | CF₃CH₂— | Et— | Me— | CH |
| 520 | o-O-i-Bu | CF₃CH₂— | Et— | Me— | CH |
| 521 | m-O-i-Bu | CF₃CH₂— | Et— | Me— | CH |
| 522 | p-O-i-Bu | CF₃CH₂— | Et— | Me— | CH |
| 523 | o-OCF₃ | CF₃CH₂— | Et— | Me— | CH |
| 524 | m-OCF₃ | CF₃CH₂— | Et— | Me— | CH |
| 525 | p-OCF₃ | CF₃CH₂— | Et— | Me— | CH |
| 526 | H | CF₃CH₂— | Et— | Me— | N |
| 527 | o-OCH₃ | CF₃CH₂— | Et— | Me— | N |
| 528 | m-OCH₃ | CF₃CH₂— | Et— | Me— | N |
| 529 | p-OCH₃ | CF₃CH₂— | Et— | Me— | N |
| 530 | o-Cl | CF₃CH₂— | Et— | Me— | N |
| 531 | m-Cl | CF₃CH₂— | Et— | Me— | N |
| 532 | p-Cl | CF₃CH₂— | Et— | Me— | N |
| 533 | o-Br | CF₃CH₂— | Et— | Me— | N |
| 534 | m-Br | CF₃CH₂— | Et— | Me— | N |
| 535 | p-Br | CF₃CH₂— | Et— | Me— | N |
| 536 | o-NH₂ | CF₃CH₂— | Et— | Me— | N |
| 537 | m-NH₂ | CF₃CH₂— | Et— | Me— | N |
| 538 | p-NH₂ | CF₃CH₂— | Et— | Me— | N |
| 539 | o-NO₂ | CF₃CH₂— | Et— | Me— | N |
| 540 | m-NO₂ | CF₃CH₂— | Et— | Me— | N |
| 541 | p-NO₂ | CF₃CH₂— | Et— | Me— | N |
| 542 | o-OC₂H₅ | CF₃CH₂— | Et— | Me— | N |
| 543 | m-OC₂H₅ | CF₃CH₂— | Et— | Me— | N |
| 544 | p-OC₂H₅ | CF₃CH₂— | Et— | Me— | N |
| 545 | p-I | CF₃CH₂— | Et— | Me— | N |
| 546 | o-O-n-Pr | CF₃CH₂— | Et— | Me— | N |
| 547 | m-O-n-Pr | CF₃CH₂— | Et— | Me— | N |
| 548 | p-O-n-Pr | CF₃CH₂— | Et— | Me— | N |
| 549 | o-O-i-Pr | CF₃CH₂— | Et— | Me— | N |
| 550 | m-O-i-Pr | CF₃CH₂— | Et— | Me— | N |
| 551 | p-O-i-Pr | CF₃CH₂— | Et— | Me— | N |
| 552 | o-O-n-Bu | CF₃CH₂— | Et— | Me— | N |
| 553 | m-O-n-Bu | CF₃CH₂— | Et— | Me— | N |
| 554 | p-O-n-Bu | CF₃CH₂— | Et— | Me— | N |
| 555 | o-O-i-Bu | CF₃CH₂— | Et— | Me— | N |
| 556 | m-O-i-Bu | CF₃CH₂— | Et— | Me— | N |
| 557 | p-O-i-Bu | CF₃CH₂— | Et— | Me— | N |
| 558 | o-OCF₃ | CF₃CH₂— | Et— | Me— | N |
| 559 | m-OCF₃ | CF₃CH₂— | Et— | Me— | N |
| 560 | p-OCF₃ | CF₃CH₂— | Et— | Me— | N |
| 561 | H | CF₃CH₂— | H | Me— | CH |
| 562 | o-OCH₃ | CF₃CH₂— | H | Me— | CH |
| 563 | m-OCH₃ | CF₃CH₂— | H | Me— | CH |
| 564 | p-OCH₃ | CF₃CH₂— | H | Me— | CH |
| 565 | o-Cl | CF₃CH₂— | H | Me— | CH |
| 566 | m-Cl | CF₃CH₂— | H | Me— | CH |
| 567 | p-Cl | CF₃CH₂— | H | Me— | CH |
| 568 | o-Br | CF₃CH₂— | H | Me— | CH |
| 569 | m-Br | CF₃CH₂— | H | Me— | CH |
| 570 | p-Br | CF₃CH₂— | H | Me— | CH |
| 571 | o-NH₂ | CF₃CH₂— | H | Me— | CH |
| 572 | m-NH₂ | CF₃CH₂— | H | Me— | CH |
| 573 | p-NH₂ | CF₃CH₂— | H | Me— | CH |
| 574 | o-NO₂ | CF₃CH₂— | H | Me— | CH |
| 575 | m-NO₂ | CF₃CH₂— | H | Me— | CH |
| 576 | p-NO₂ | CF₃CH₂— | H | Me— | CH |
| 577 | o-OC₂H₅ | CF₃CH₂— | H | Me— | CH |
| 578 | m-OC₂H₅ | CF₃CH₂— | H | Me— | CH |
| 579 | p-OC₂H₅ | CF₃CH₂— | H | Me— | CH |
| 580 | p-I | CF₃CH₂— | H | Me— | CH |
| 581 | o-O-n-Pr | CF₃CH₂— | H | Me— | CH |
| 582 | m-O-n-Pr | CF₃CH₂— | H | Me— | CH |
| 583 | p-O-n-Pr | CF₃CH₂— | H | Me— | CH |
| 584 | o-O-i-Pr | CF₃CH₂— | H | Me— | CH |
| 585 | m-O-i-Pr | CF₃CH₂— | H | Me— | CH |
| 586 | p-O-i-Pr | CF₃CH₂— | H | Me— | CH |
| 587 | o-O-n-Bu | CF₃CH₂— | H | Me— | CH |
| 588 | m-O-n-Bu | CF₃CH₂— | H | Me— | CH |
| 589 | p-O-n-Bu | CF₃CH₂— | H | Me— | CH |
| 590 | o-O-i-Bu | CF₃CH₂— | H | Me— | CH |
| 591 | m-O-i-Bu | CF₃CH₂— | H | Me— | CH |
| 592 | p-O-i-Bu | CF₃CH₂— | H | Me— | CH |
| 593 | o-OCF₃ | CF₃CH₂— | H | Me— | CH |
| 594 | m-OCF₃ | CF₃CH₂— | H | Me— | CH |
| 595 | p-OCF₃ | CF₃CH₂— | H | Me— | CH |
| 596 | H | CF₃CH₂— | H | Me— | N |
| 597 | o-OCH₃ | CF₃CH₂— | H | Me— | N |
| 598 | m-OCH₃ | CF₃CH₂— | H | Me— | N |
| 599 | p-OCH₃ | CF₃CH₂— | H | Me— | N |
| 600 | o-Cl | CF₃CH₂— | H | Me— | N |
| 601 | m-Cl | CF₃CH₂— | H | Me— | N |
| 602 | p-Cl | CF₃CH₂— | H | Me— | N |
| 603 | o-Br | CF₃CH₂— | H | Me— | N |
| 604 | m-Br | CF₃CH₂— | H | Me— | N |
| 605 | p-Br | CF₃CH₂— | H | Me— | N |
| 606 | o-NH₂ | CF₃CH₂— | H | Me— | N |
| 607 | m-NH₂ | CF₃CH₂— | H | Me— | N |
| 608 | p-NH₂ | CF₃CH₂— | H | Me— | N |
| 609 | o-NO₂ | CF₃CH₂— | H | Me— | N |
| 610 | m-NO₂ | CF₃CH₂— | H | Me— | N |
| 611 | p-NO₂ | CF₃CH₂— | H | Me— | N |
| 612 | o-OC₂H₅ | CF₃CH₂— | H | Me— | N |
| 613 | m-OC₂H₅ | CF₃CH₂— | H | Me— | N |
| 614 | p-OC₂H₅ | CF₃CH₂— | H | Me— | N |
| 615 | p-I | CF₃CH₂— | H | Me— | N |
| 616 | o-O-n-Pr | CF₃CH₂— | H | Me— | N |
| 617 | m-O-n-Pr | CF₃CH₂— | H | Me— | N |
| 618 | p-O-n-Pr | CF₃CH₂— | H | Me— | N |
| 619 | o-O-i-Pr | CF₃CH₂— | H | Me— | N |
| 620 | m-O-i-Pr | CF₃CH₂— | H | Me— | N |
| 621 | p-O-i-Pr | CF₃CH₂— | H | Me— | N |
| 622 | o-O-n-Bu | CF₃CH₂— | H | Me— | N |
| 623 | m-O-n-Bu | CF₃CH₂— | H | Me— | N |
| 624 | p-O-n-Bu | CF₃CH₂— | H | Me— | N |
| 625 | o-O-i-Bu | CF₃CH₂— | H | Me— | N |
| 626 | m-O-i-Bu | CF₃CH₂— | H | Me— | N |
| 627 | p-O-i-Bu | CF₃CH₂— | H | Me— | N |
| 628 | o-OCF₃ | CF₃CH₂— | H | Me— | N |
| 629 | m-OCF₃ | CF₃CH₂— | H | Me— | N |
| 630 | p-OCF₃ | CF₃CH₂— | H | Me— | N |
| 631 | H | H | H | Me— | CH |
| 632 | o-OCH₃ | H | H | Me— | CH |
| 633 | m-OCH₃ | H | H | Me— | CH |
| 634 | p-OCH₃ | H | H | Me— | CH |
| 635 | o-Cl | H | H | Me— | CH |
| 636 | m-Cl | H | H | Me— | CH |
| 637 | p-Cl | H | H | Me— | CH |
| 638 | o-Br | H | H | Me— | CH |
| 639 | m-Br | H | H | Me— | CH |
| 640 | p-Br | H | H | Me— | CH |
| 641 | o-NH₂ | H | H | Me— | CH |
| 642 | m-NH₂ | H | H | Me— | CH |
| 643 | p-NH₂ | H | H | Me— | CH |
| 644 | o-NO₂ | H | H | Me— | CH |
| 645 | m-NO₂ | H | H | Me— | CH |
| 646 | p-NO₂ | H | H | Me— | CH |
| 647 | o-OC₂H₅ | H | H | Me— | CH |
| 648 | m-OC₂H₅ | H | H | Me— | CH |
| 649 | p-OC₂H₅ | H | H | Me— | CH |
| 650 | p-I | H | H | Me— | CH |
| 651 | o-O-n-Pr | H | H | Me— | CH |
| 652 | m-O-n-Pr | H | H | Me— | CH |
| 653 | p-O-n-Pr | H | H | Me— | CH |
| 654 | o-O-i-Pr | H | H | Me— | CH |
| 655 | m-O-i-Pr | H | H | Me— | CH |
| 656 | p-O-i-Pr | H | H | Me— | CH |
| 657 | o-O-n-Bu | H | H | Me— | CH |
| 658 | m-O-n-Bu | H | H | Me— | CH |
| 659 | p-O-n-Bu | H | H | Me— | CH |
| 660 | o-O-i-Bu | H | H | Me— | CH |
| 661 | m-O-i-Bu | H | H | Me— | CH |
| 662 | p-O-i-Bu | H | H | Me— | CH |
| 663 | o-OCF₃ | H | H | Me— | CH |
| 664 | m-OCF₃ | H | H | Me— | CH |
| 665 | p-OCF₃ | H | H | Me— | CH |
| 666 | H | H | H | Me— | N |
| 667 | o-OCH₃ | H | H | Me— | N |
| 668 | m-OCH₃ | H | H | Me— | N |
| 669 | p-OCH₃ | H | H | Me— | N |
| 670 | o-Cl | H | H | Me— | N |
| 671 | m-Cl | H | H | Me— | N |
| 672 | p-Cl | H | H | Me— | N |
| 673 | o-Br | H | H | Me— | N |
| 674 | m-Br | H | H | Me— | N |
| 675 | p-Br | H | H | Me— | N |
| 676 | o-NH₂ | H | H | Me— | N |
| 677 | m-NH₂ | H | H | Me— | N |
| 678 | p-NH₂ | H | H | Me— | N |
| 679 | o-NO₂ | H | H | Me— | N |
| 680 | m-NO₂ | H | H | Me— | N |
| 681 | p-NO₂ | H | H | Me— | N |
| 682 | o-OC₂H₅ | H | H | Me— | N |
| 683 | m-OC₂H₅ | H | H | Me— | N |
| 684 | p-OC₂H₅ | H | H | Me— | N |
| 685 | p-I | H | H | Me— | N |
| 686 | o-O-n-Pr | H | H | Me— | N |
| 687 | m-O-n-Pr | H | H | Me— | N |
| 688 | p-O-n-Pr | H | H | Me— | N |
| 689 | o-O-i-Pr | H | H | Me— | N |
| 690 | m-O-i-Pr | H | H | Me— | N |
| 691 | p-O-i-Pr | H | H | Me— | N |
| 692 | o-O-n-Bu | H | H | Me— | N |
| 693 | m-O-n-Bu | H | H | Me— | N |
| 694 | p-O-n-Bu | H | H | Me— | N |
| 695 | o-O-i-Bu | H | H | Me— | N |
| 696 | m-O-i-Bu | H | H | Me— | N |
| 697 | p-O-i-Bu | H | H | Me— | N |
| 698 | o-OCF₃ | H | H | Me— | N |
| 699 | m-OCF₃ | H | H | Me— | N |
| 700 | p-OCF₃ | H | H | Me— | N |
| 701 | H | CF₃CH₂— | CF₃CH₂— | —CH₂F | CH |
| 702 | o-OCH₃ | CF₃CH₂— | CF₃CH₂— | —CH₂F | CH |
| 703 | m-OCH₃ | CF₃CH₂— | CF₃CH₂— | —CH₂F | CH |
| 704 | p-OCH₃ | CF₃CH₂— | CF₃CH₂— | —CH₂F | CH |
| 705 | o-Cl | CF₃CH₂— | CF₃CH₂— | —CH₂F | CH |
| 706 | m-Cl | CF₃CH₂— | CF₃CH₂— | —CH₂F | CH |
| 707 | p-Cl | CF₃CH₂— | CF₃CH₂— | —CH₂F | CH |
| 708 | o-Br | CF₃CH₂— | CF₃CH₂— | —CH₂F | CH |
| 709 | m-Br | CF₃CH₂— | CF₃CH₂— | —CH₂F | CH |
| 710 | p-Br | CF₃CH₂— | CF₃CH₂— | —CH₂F | CH |
| 711 | o-NH₂ | CF₃CH₂— | CF₃CH₂— | —CH₂F | CH |
| 712 | m-NH₂ | CF₃CH₂— | CF₃CH₂— | —CH₂F | CH |
| 713 | p-NH₂ | CF₃CH₂— | CF₃CH₂— | —CH₂F | CH |
| 714 | o-NO₂ | CF₃CH₂— | CF₃CH₂— | —CH₂F | CH |
| 715 | m-NO₂ | CF₃CH₂— | CF₃CH₂— | —CH₂F | CH |
| 716 | p-NO₂ | CF₃CH₂— | CF₃CH₂— | —CH₂F | CH |
| 717 | o-OC₂H₅ | CF₃CH₂— | CF₃CH₂— | —CH₂F | CH |
| 718 | m-OC₂H₅ | CF₃CH₂— | CF₃CH₂— | —CH₂F | CH |
| 719 | p-OC₂H₅ | CF₃CH₂— | CF₃CH₂— | —CH₂F | CH |
| 720 | p-I | CF₃CH₂— | CF₃CH₂— | —CH₂F | CH |
| 721 | o-O-n-Pr | CF₃CH₂— | CF₃CH₂— | —CH₂F | CH |
| 722 | m-O-n-Pr | CF₃CH₂— | CF₃CH₂— | —CH₂F | CH |
| 723 | p-O-n-Pr | CF₃CH₂— | CF₃CH₂— | —CH₂F | CH |
| 724 | o-O-i-Pr | CF₃CH₂— | CF₃CH₂— | —CH₂F | CH |
| 725 | m-O-i-Pr | CF₃CH₂— | CF₃CH₂— | —CH₂F | CH |
| 726 | p-O-i-Pr | CF₃CH₂— | CF₃CH₂— | —CH₂F | CH |
| 727 | o-O-n-Bu | CF₃CH₂— | CF₃CH₂— | —CH₂F | CH |
| 728 | m-O-n-Bu | CF₃CH₂— | CF₃CH₂— | —CH₂F | CH |
| 729 | p-O-n-Bu | CF₃CH₂— | CF₃CH₂— | —CH₂F | CH |
| 730 | o-O-i-Bu | CF₃CH₂— | CF₃CH₂— | —CH₂F | CH |
| 731 | m-O-i-Bu | CF₃CH₂— | CF₃CH₂— | —CH₂F | CH |
| 732 | p-O-i-Bu | CF₃CH₂— | CF₃CH₂— | —CH₂F | CH |
| 733 | o-OCF₃ | CF₃CH₂— | CF₃CH₂— | —CH₂F | CH |
| 734 | m-OCF₃ | CF₃CH₂— | CF₃CH₂— | —CH₂F | CH |
| 735 | p-OCF₃ | CF₃CH₂— | CF₃CH₂— | —CH₂F | CH |
| 736 | H | CF₃CH₂— | CF₃CH₂— | —CH₂F | N |
| 737 | o-OCH₃ | CF₃CH₂— | CF₃CH₂— | —CH₂F | N |
| 738 | m-OCH₃ | CF₃CH₂— | CF₃CH₂— | —CH₂F | N |
| 739 | p-OCH₃ | CF₃CH₂— | CF₃CH₂— | —CH₂F | N |
| 740 | o-Cl | CF₃CH₂— | CF₃CH₂— | —CH₂F | N |
| 741 | m-Cl | CF₃CH₂— | CF₃CH₂— | —CH₂F | N |
| 742 | p-Cl | CF₃CH₂— | CF₃CH₂— | —CH₂F | N |
| 743 | o-Br | CF₃CH₂— | CF₃CH₂— | —CH₂F | N |
| 744 | m-Br | CF₃CH₂— | CF₃CH₂— | —CH₂F | N |
| 745 | p-Br | CF₃CH₂— | CF₃CH₂— | —CH₂F | N |
| 746 | o-NH₂ | CF₃CH₂— | CF₃CH₂— | —CH₂F | N |
| 747 | m-NH₂ | CF₃CH₂— | CF₃CH₂— | —CH₂F | N |
| 748 | p-NH₂ | CF₃CH₂— | CF₃CH₂— | —CH₂F | N |
| 749 | o-NO₂ | CF₃CH₂— | CF₃CH₂— | —CH₂F | N |
| 750 | m-NO₂ | CF₃CH₂— | CF₃CH₂— | —CH₂F | N |
| 751 | p-NO₂ | CF₃CH₂— | CF₃CH₂— | —CH₂F | N |
| 752 | o-OC₂H₅ | CF₃CH₂— | CF₃CH₂— | —CH₂F | N |
| 753 | m-OC₂H₅ | CF₃CH₂— | CF₃CH₂— | —CH₂F | N |
| 754 | p-OC₂H₅ | CF₃CH₂— | CF₃CH₂— | —CH₂F | N |
| 755 | p-I | CF₃CH₂— | CF₃CH₂— | —CH₂F | N |
| 756 | o-O-n-Pr | CF₃CH₂— | CF₃CH₂— | —CH₂F | N |
| 757 | m-O-n-Pr | CF₃CH₂— | CF₃CH₂— | —CH₂F | N |
| 758 | p-O-n-Pr | CF₃CH₂— | CF₃CH₂— | —CH₂F | N |
| 759 | o-O-i-Pr | CF₃CH₂— | CF₃CH₂— | —CH₂F | N |
| 760 | m-O-i-Pr | CF₃CH₂— | CF₃CH₂— | —CH₂F | N |
| 761 | p-O-i-Pr | CF₃CH₂— | CF₃CH₂— | —CH₂F | N |
| 762 | o-O-n-Bu | CF₃CH₂— | CF₃CH₂— | —CH₂F | N |
| 763 | m-O-n-Bu | CF₃CH₂— | CF₃CH₂— | —CH₂F | N |
| 764 | p-O-n-Bu | CF₃CH₂— | CF₃CH₂— | —CH₂F | N |
| 765 | o-O-i-Bu | CF₃CH₂— | CF₃CH₂— | —CH₂F | N |
| 766 | m-O-i-Bu | CF₃CH₂— | CF₃CH₂— | —CH₂F | N |
| 767 | p-O-i-Bu | CF₃CH₂— | CF₃CH₂— | —CH₂F | N |
| 768 | o-OCF₃ | CF₃CH₂— | CF₃CH₂— | —CH₂F | N |
| 769 | m-OCF₃ | CF₃CH₂— | CF₃CH₂— | —CH₂F | N |
| 770 | p-OCF₃ | CF₃CH₂— | CF₃CH₂— | —CH₂F | N |
| 771 | H | CF₃CH₂— | Me— | —CH₂F | CH |
| 772 | o-OCH₃ | CF₃CH₂— | Me— | —CH₂F | CH |
| 773 | m-OCH₃ | CF₃CH₂— | Me— | —CH₂F | CH |
| 774 | p-OCH₃ | CF₃CH₂— | Me— | —CH₂F | CH |
| 775 | o-Cl | CF₃CH₂— | Me— | —CH₂F | CH |
| 776 | m-Cl | CF₃CH₂— | Me— | —CH₂F | CH |
| 777 | p-Cl | CF₃CH₂— | Me— | —CH₂F | CH |
| 778 | o-Br | CF₃CH₂— | Me— | —CH₂F | CH |
| 779 | m-Br | CF₃CH₂— | Me— | —CH₂F | CH |
| 780 | p-Br | CF₃CH₂— | Me— | —CH₂F | CH |
| 781 | o-NH₂ | CF₃CH₂— | Me— | —CH₂F | CH |
| 782 | m-NH₂ | CF₃CH₂— | Me— | —CH₂F | CH |
| 783 | p-NH₂ | CF₃CH₂— | Me— | —CH₂F | CH |
| 784 | o-NO₂ | CF₃CH₂— | Me— | —CH₂F | CH |
| 785 | m-NO₂ | CF₃CH₂— | Me— | —CH₂F | CH |
| 786 | p-NO₂ | CF₃CH₂— | Me— | —CH₂F | CH |
| 787 | o-OC₂H₅ | CF₃CH₂— | Me— | —CH₂F | CH |
| 788 | m-OC₂H₅ | CF₃CH₂— | Me— | —CH₂F | CH |
| 789 | p-OC₂H₅ | CF₃CH₂— | Me— | —CH₂F | CH |
| 790 | p-I | CF₃CH₂— | Me— | —CH₂F | CH |
| 791 | o-O-n-Pr | CF₃CH₂— | Me— | —CH₂F | CH |
| 792 | m-O-n-Pr | CF₃CH₂— | Me— | —CH₂F | CH |
| 793 | p-O-n-Pr | CF₃CH₂— | Me— | —CH₂F | CH |
| 794 | o-O-i-Pr | CF₃CH₂— | Me— | —CH₂F | CH |
| 795 | m-O-i-Pr | CF₃CH₂— | Me— | —CH₂F | CH |
| 796 | p-O-i-Pr | CF₃CH₂— | Me— | —CH₂F | CH |
| 797 | o-O-n-Bu | CF₃CH₂— | Me— | —CH₂F | CH |
| 798 | m-O-n-Bu | CF₃CH₂— | Me— | —CH₂F | CH |
| 799 | p-O-n-Bu | CF₃CH₂— | Me— | —CH₂F | CH |
| 800 | o-O-i-Bu | CF₃CH₂— | Me— | —CH₂F | CH |
| 801 | m-O-i-Bu | CF₃CH₂— | Me— | —CH₂F | CH |
| 802 | p-O-i-Bu | CF₃CH₂— | Me— | —CH₂F | CH |
| 803 | o-OCF₃ | CF₃CH₂— | Me— | —CH₂F | CH |
| 804 | m-OCF₃ | CF₃CH₂— | Me— | —CH₂F | CH |
| 805 | p-OCF₃ | CF₃CH₂— | Me— | —CH₂F | CH |
| 806 | H | CF₃CH₂— | Me— | —CH₂F | N |
| 807 | o-OCH₃ | CF₃CH₂— | Me— | —CH₂F | N |
| 808 | m-OCH₃ | CF₃CH₂— | Me— | —CH₂F | N |
| 809 | p-OCH₃ | CF₃CH₂— | Me— | —CH₂F | N |
| 810 | o-Cl | CF₃CH₂— | Me— | —CH₂F | N |
| 811 | m-Cl | CF₃CH₂— | Me— | —CH₂F | N |
| 812 | p-Cl | CF₃CH₂— | Me— | —CH₂F | N |
| 813 | o-Br | CF₃CH₂— | Me— | —CH₂F | N |
| 814 | m-Br | CF₃CH₂— | Me— | —CH₂F | N |
| 815 | p-Br | CF₃CH₂— | Me— | —CH₂F | N |
| 816 | o-NH₂ | CF₃CH₂— | Me— | —CH₂F | N |
| 817 | m-NH₂ | CF₃CH₂— | Me— | —CH₂F | N |
| 818 | p-NH₂ | CF₃CH₂— | Me— | —CH₂F | N |
| 819 | o-NO₂ | CF₃CH₂— | Me— | —CH₂F | N |
| 820 | m-NO₂ | CF₃CH₂— | Me— | —CH₂F | N |
| 821 | p-NO₂ | CF₃CH₂— | Me— | —CH₂F | N |
| 822 | o-OC₂H₅ | CF₃CH₂— | Me— | —CH₂F | N |
| 823 | m-OC₂H₅ | CF₃CH₂— | Me— | —CH₂F | N |
| 824 | p-OC₂H₅ | CF₃CH₂— | Me— | —CH₂F | N |
| 825 | p-I | CF₃CH₂— | Me— | —CH₂F | N |
| 826 | o-O-n-Pr | CF₃CH₂— | Me— | —CH₂F | N |
| 827 | m-O-n-Pr | CF₃CH₂— | Me— | —CH₂F | N |
| 828 | p-O-n-Pr | CF₃CH₂— | Me— | —CH₂F | N |
| 829 | o-O-i-Pr | CF₃CH₂— | Me— | —CH₂F | N |
| 830 | m-O-i-Pr | CF₃CH₂— | Me— | —CH₂F | N |
| 831 | p-O-i-Pr | CF₃CH₂— | Me— | —CH₂F | N |
| 832 | o-O-n-Bu | CF₃CH₂— | Me— | —CH₂F | N |
| 833 | m-O-n-Bu | CF₃CH₂— | Me— | —CH₂F | N |
| 834 | p-O-n-Bu | CF₃CH₂— | Me— | —CH₂F | N |
| 835 | o-O-i-Bu | CF₃CH₂— | Me— | —CH₂F | N |
| 836 | m-O-i-Bu | CF₃CH₂— | Me— | —CH₂F | N |
| 837 | p-O-i-Bu | CF₃CH₂— | Me— | —CH₂F | N |
| 838 | o-OCF₃ | CF₃CH₂— | Me— | —CH₂F | N |
| 839 | m-OCF₃ | CF₃CH₂— | Me— | —CH₂F | N |
| 840 | p-OCF₃ | CF₃CH₂— | Me— | —CH₂F | N |
| 841 | H | CF₃CH₂— | Et— | —CH₂F | CH |
| 842 | o-OCH₃ | CF₃CH₂— | Et— | —CH₂F | CH |
| 843 | m-OCH₃ | CF₃CH₂— | Et— | —CH₂F | CH |
| 844 | p-OCH₃ | CF₃CH₂— | Et— | —CH₂F | CH |
| 845 | o-Cl | CF₃CH₂— | Et— | —CH₂F | CH |
| 846 | m-Cl | CF₃CH₂— | Et— | —CH₂F | CH |
| 847 | p-Cl | CF₃CH₂— | Et— | —CH₂F | CH |
| 848 | o-Br | CF₃CH₂— | Et— | —CH₂F | CH |
| 849 | m-Br | CF₃CH₂— | Et— | —CH₂F | CH |
| 850 | p-Br | CF₃CH₂— | Et— | —CH₂F | CH |
| 851 | o-NH₂ | CF₃CH₂— | Et— | —CH₂F | CH |
| 852 | m-NH₂ | CF₃CH₂— | Et— | —CH₂F | CH |
| 853 | p-NH₂ | CF₃CH₂— | Et— | —CH₂F | CH |
| 854 | o-NO₂ | CF₃CH₂— | Et— | —CH₂F | CH |
| 855 | m-NO₂ | CF₃CH₂— | Et— | —CH₂F | CH |
| 856 | p-NO₂ | CF₃CH₂— | Et— | —CH₂F | CH |
| 857 | o-OC₂H₅ | CF₃CH₂— | Et— | —CH₂F | CH |
| 858 | m-OC₂H₅ | CF₃CH₂— | Et— | —CH₂F | CH |
| 859 | p-OC₂H₅ | CF₃CH₂— | Et— | —CH₂F | CH |
| 860 | p-I | CF₃CH₂— | Et— | —CH₂F | CH |
| 861 | o-O-n-Pr | CF₃CH₂— | Et— | —CH₂F | CH |
| 862 | m-O-n-Pr | CF₃CH₂— | Et— | —CH₂F | CH |
| 863 | p-O-n-Pr | CF₃CH₂— | Et— | —CH₂F | CH |
| 864 | o-O-i-Pr | CF₃CH₂— | Et— | —CH₂F | CH |
| 865 | m-O-i-Pr | CF₃CH₂— | Et— | —CH₂F | CH |
| 866 | p-O-i-Pr | CF₃CH₂— | Et— | —CH₂F | CH |
| 867 | o-O-n-Bu | CF₃CH₂— | Et— | —CH₂F | CH |
| 868 | m-O-n-Bu | CF₃CH₂— | Et— | —CH₂F | CH |
| 869 | p-O-n-Bu | CF₃CH₂— | Et— | —CH₂F | CH |
| 870 | o-O-i-Bu | CF₃CH₂— | Et— | —CH₂F | CH |
| 871 | m-O-i-Bu | CF₃CH₂— | Et— | —CH₂F | CH |
| 872 | p-O-i-Bu | CF₃CH₂— | Et— | —CH₂F | CH |
| 873 | o-OCF₃ | CF₃CH₂— | Et— | —CH₂F | CH |
| 874 | m-OCF₃ | CF₃CH₂— | Et— | —CH₂F | CH |
| 875 | p-OCF₃ | CF₃CH₂— | Et— | —CH₂F | CH |
| 876 | H | CF₃CH₂— | Et— | —CH₂F | N |
| 877 | o-OCH₃ | CF₃CH₂— | Et— | —CH₂F | N |
| 878 | m-OCH₃ | CF₃CH₂— | Et— | —CH₂F | N |
| 879 | p-OCH₃ | CF₃CH₂— | Et— | —CH₂F | N |
| 880 | o-Cl | CF₃CH₂— | Et— | —CH₂F | N |
| 881 | m-Cl | CF₃CH₂— | Et— | —CH₂F | N |
| 882 | p-Cl | CF₃CH₂— | Et— | —CH₂F | N |
| 883 | o-Br | CF₃CH₂— | Et— | —CH₂F | N |
| 884 | m-Br | CF₃CH₂— | Et— | —CH₂F | N |
| 885 | p-Br | CF₃CH₂— | Et— | —CH₂F | N |
| 886 | o-NH₂ | CF₃CH₂— | Et— | —CH₂F | N |
| 887 | m-NH₂ | CF₃CH₂— | Et— | —CH₂F | N |
| 888 | p-NH₂ | CF₃CH₂— | Et— | —CH₂F | N |
| 889 | o-NO₂ | CF₃CH₂— | Et— | —CH₂F | N |
| 890 | m-NO₂ | CF₃CH₂— | Et— | —CH₂F | N |
| 891 | p-NO₂ | CF₃CH₂— | Et— | —CH₂F | N |
| 892 | o-OC₂H₅ | CF₃CH₂— | Et— | —CH₂F | N |
| 893 | m-OC₂H₅ | CF₃CH₂— | Et— | —CH₂F | N |
| 894 | p-OC₂H₅ | CF₃CH₂— | Et— | —CH₂F | N |
| 895 | p-I | CF₃CH₂— | Et— | —CH₂F | N |
| 896 | o-O-n-Pr | CF₃CH₂— | Et— | —CH₂F | N |
| 897 | m-O-n-Pr | CF₃CH₂— | Et— | —CH₂F | N |
| 898 | p-O-n-Pr | CF₃CH₂— | Et— | —CH₂F | N |
| 899 | o-O-i-Pr | CF₃CH₂— | Et— | —CH₂F | N |
| 900 | m-O-i-Pr | CF₃CH₂— | Et— | —CH₂F | N |
| 901 | p-O-i-Pr | CF₃CH₂— | Et— | —CH₂F | N |
| 902 | o-O-n-Bu | CF₃CH₂— | Et— | —CH₂F | N |
| 903 | m-O-n-Bu | CF₃CH₂— | Et— | —CH₂F | N |
| 904 | p-O-n-Bu | CF₃CH₂— | Et— | —CH₂F | N |
| 905 | o-O-i-Bu | CF₃CH₂— | Et— | —CH₂F | N |
| 906 | m-O-i-Bu | CF₃CH₂— | Et— | —CH₂F | N |
| 907 | p-O-i-Bu | CF₃CH₂— | Et— | —CH₂F | N |
| 908 | o-OCF₃ | CF₃CH₂— | Et— | —CH₂F | N |
| 909 | m-OCF₃ | CF₃CH₂— | Et— | —CH₂F | N |
| 910 | p-OCF₃ | CF₃CH₂— | Et— | —CH₂F | N |
| 911 | H | CF₃CH₂— | H | —CH₂F | CH |
| 912 | o-OCH₃ | CF₃CH₂— | H | —CH₂F | CH |
| 913 | m-OCH₃ | CF₃CH₂— | H | —CH₂F | CH |
| 914 | p-OCH₃ | CF₃CH₂— | H | —CH₂F | CH |
| 915 | o-Cl | CF₃CH₂— | H | —CH₂F | CH |
| 916 | m-Cl | CF₃CH₂— | H | —CH₂F | CH |
| 917 | p-Cl | CF₃CH₂— | H | —CH₂F | CH |
| 918 | o-Br | CF₃CH₂— | H | —CH₂F | CH |
| 919 | m-Br | CF₃CH₂— | H | —CH₂F | CH |
| 920 | p-Br | CF₃CH₂— | H | —CH₂F | CH |
| 921 | o-NH₂ | CF₃CH₂— | H | —CH₂F | CH |
| 922 | m-NH₂ | CF₃CH₂— | H | —CH₂F | CH |
| 923 | p-NH₂ | CF₃CH₂— | H | —CH₂F | CH |
| 924 | o-NO₂ | CF₃CH₂— | H | —CH₂F | CH |
| 925 | m-NO₂ | CF₃CH₂— | H | —CH₂F | CH |
| 926 | p-NO₂ | CF₃CH₂— | H | —CH₂F | CH |
| 927 | o-OC₂H₅ | CF₃CH₂— | H | —CH₂F | CH |
| 928 | m-OC₂H₅ | CF₃CH₂— | H | —CH₂F | CH |
| 929 | p-OC₂H₅ | CF₃CH₂— | H | —CH₂F | CH |
| 930 | p-I | CF₃CH₂— | H | —CH₂F | CH |
| 931 | o-O-n-Pr | CF₃CH₂— | H | —CH₂F | CH |
| 932 | m-O-n-Pr | CF₃CH₂— | H | —CH₂F | CH |
| 933 | p-O-n-Pr | CF₃CH₂— | H | —CH₂F | CH |
| 934 | o-O-i-Pr | CF₃CH₂— | H | —CH₂F | CH |
| 935 | m-O-i-Pr | CF₃CH₂— | H | —CH₂F | CH |
| 936 | p-O-i-Pr | CF₃CH₂— | H | —CH₂F | CH |
| 937 | o-O-n-Bu | CF₃CH₂— | H | —CH₂F | CH |
| 938 | m-O-n-Bu | CF₃CH₂— | H | —CH₂F | CH |
| 939 | p-O-n-Bu | CF₃CH₂— | H | —CH₂F | CH |
| 940 | o-O-i-Bu | CF₃CH₂— | H | —CH₂F | CH |
| 941 | m-O-i-Bu | CF₃CH₂— | H | —CH₂F | CH |
| 942 | p-O-i-Bu | CF₃CH₂— | H | —CH₂F | CH |
| 943 | o-OCF₃ | CF₃CH₂— | H | —CH₂F | CH |
| 944 | m-OCF₃ | CF₃CH₂— | H | —CH₂F | CH |
| 945 | p-OCF₃ | CF₃CH₂— | H | —CH₂F | CH |
| 946 | H | CF₃CH₂— | H | —CH₂F | N |
| 947 | o-OCH₃ | CF₃CH₂— | H | —CH₂F | N |
| 948 | m-OCH₃ | CF₃CH₂— | H | —CH₂F | N |
| 949 | p-OCH₃ | CF₃CH₂— | H | —CH₂F | N |
| 950 | o-Cl | CF₃CH₂— | H | —CH₂F | N |
| 951 | m-Cl | CF₃CH₂— | H | —CH₂F | N |
| 952 | p-Cl | CF₃CH₂— | H | —CH₂F | N |
| 953 | o-Br | CF₃CH₂— | H | —CH₂F | N |
| 954 | m-Br | CF₃CH₂— | H | —CH₂F | N |
| 955 | p-Br | CF₃CH₂— | H | —CH₂F | N |
| 956 | o-NH₂ | CF₃CH₂— | H | —CH₂F | N |
| 957 | m-NH₂ | CF₃CH₂— | H | —CH₂F | N |
| 958 | p-NH₂ | CF₃CH₂— | H | —CH₂F | N |
| 959 | o-NO₂ | CF₃CH₂— | H | —CH₂F | N |
| 960 | m-NO₂ | CF₃CH₂— | H | —CH₂F | N |
| 961 | p-NO₂ | CF₃CH₂— | H | —CH₂F | N |
| 962 | o-OC₂H₅ | CF₃CH₂— | H | —CH₂F | N |
| 963 | m-OC₂H₅ | CF₃CH₂— | H | —CH₂F | N |
| 964 | p-OC₂H₅ | CF₃CH₂— | H | —CH₂F | N |
| 965 | p-I | CF₃CH₂— | H | —CH₂F | N |
| 966 | o-O-n-Pr | CF₃CH₂— | H | —CH₂F | N |
| 967 | m-O-n-Pr | CF₃CH₂— | H | —CH₂F | N |
| 968 | p-O-n-Pr | CF₃CH₂— | H | —CH₂F | N |
| 969 | o-O-i-Pr | CF₃OH₂- | H | —CH₂F | N |
| 970 | m-O-i-Pr | CF₃CH₂— | H | —CH₂F | N |
| 971 | p-O-i-Pr | CF₃CH₂— | H | —CH₂F | N |
| 972 | o-O-n-Bu | CF₃CH₂— | H | —CH₂F | N |
| 973 | m-O-n-Bu | CF₃CH₂— | H | —CH₂F | N |
| 974 | p-O-n-Bu | CF₃CH₂— | H | —CH₂F | N |
| 975 | o-O-i-Bu | CF₃CH₂— | H | —CH₂F | N |
| 976 | m-O-i-Bu | CF₃CH₂— | H | —CH₂F | N |
| 977 | p-O-i-Bu | CF₃CH₂— | H | —CH₂F | N |
| 978 | o-OCF₃ | CF₃CH₂— | H | —CH₂F | N |
| 979 | m-OCF₃ | CF₃CH₂— | H | —CH₂F | N |
| 980 | p-OCF₃ | CF₃CH₂— | H | —CH₂F | N |
| 981 | H | H | H | —CH₂F | CH |
| 982 | o-OCH₃ | H | H | —CH₂F | CH |
| 983 | m-OCH₃ | H | H | —CH₂F | CH |
| 984 | p-OCH₃ | H | H | —CH₂F | CH |
| 985 | o-Cl | H | H | —CH₂F | CH |
| 986 | m-Cl | H | H | —CH₂F | CH |
| 987 | p-Cl | H | H | —CH₂F | CH |
| 988 | o-Br | H | H | —CH₂F | CH |
| 989 | m-Br | H | H | —CH₂F | CH |
| 990 | p-Br | H | H | —CH₂F | CH |
| 991 | o-NH₂ | H | H | —CH₂F | CH |
| 992 | m-NH₂ | H | H | —CH₂F | CH |
| 993 | p-NH₂ | H | H | —CH₂F | CH |
| 994 | o-NO₂ | H | H | —CH₂F | CH |
| 995 | m-NO₂ | H | H | —CH₂F | CH |
| 996 | p-NO₂ | H | H | —CH₂F | CH |
| 997 | o-OC₂H₅ | H | H | —CH₂F | CH |
| 998 | m-OC₂H₅ | H | H | —CH₂F | CH |
| 999 | p-OC₂H₅ | H | H | —CH₂F | CH |
| 1000 | p-I | H | H | —CH₂F | CH |
| 1001 | o-O-n-Pr | H | H | —CH₂F | CH |
| 1002 | m-O-n-Pr | H | H | —CH₂F | CH |
| 1003 | p-O-n-Pr | H | H | —CH₂F | CH |
| 1004 | o-O-i-Pr | H | H | —CH₂F | CH |
| 1005 | m-O-i-Pr | H | H | —CH₂F | CH |
| 1006 | p-O-i-Pr | H | H | —CH₂F | CH |
| 1007 | o-O-n-Bu | H | H | —CH₂F | CH |
| 1008 | m-O-n-Bu | H | H | —CH₂F | CH |
| 1009 | p-O-n-Bu | H | H | —CH₂F | CH |
| 1010 | o-O-i-Bu | H | H | —CH₂F | CH |
| 1011 | m-O-i-Bu | H | H | —CH₂F | CH |
| 1012 | p-O-i-Bu | H | H | —CH₂F | CH |
| 1013 | o-OCF₃ | H | H | —CH₂F | CH |
| 1014 | m-OCF₃ | H | H | —CH₂F | CH |
| 1015 | p-OCF₃ | H | H | —CH₂F | CH |
| 1016 | H | H | H | —CH₂F | N |
| 1017 | o-OCH₃ | H | H | —CH₂F | N |
| 1018 | m-OCH₃ | H | H | —CH₂F | N |
| 1019 | p-OCH₃ | H | H | —CH₂F | N |
| 1020 | o-Cl | H | H | —CH₂F | N |
| 1021 | m-Cl | H | H | —CH₂F | N |
| 1022 | p-Cl | H | H | —CH₂F | N |
| 1023 | o-Br | H | H | —CH₂F | N |
| 1024 | m-Br | H | H | —CH₂F | N |
| 1025 | p-Br | H | H | —CH₂F | N |
| 1026 | o-NH₂ | H | H | —CH₂F | N |
| 1027 | m-NH₂ | H | H | —CH₂F | N |
| 1028 | p-NH₂ | H | H | —CH₂F | N |
| 1029 | o-NO₂ | H | H | —CH₂F | N |
| 1030 | m-NO₂ | H | H | —CH₂F | N |
| 1031 | p-NO₂ | H | H | —CH₂F | N |
| 1032 | o-OC₂H₅ | H | H | —CH₂F | N |
| 1033 | m-OC₂H₅ | H | H | —CH₂F | N |
| 1034 | p-OC₂H₅ | H | H | —CH₂F | N |
| 1035 | p-I | H | H | —CH₂F | N |
| 1036 | o-O-n-Pr | H | H | —CH₂F | N |
| 1037 | m-O-n-Pr | H | H | —CH₂F | N |
| 1038 | p-O-n-Pr | H | H | —CH₂F | N |
| 1039 | o-O-i-Pr | H | H | —CH₂F | N |
| 1040 | m-O-i-Pr | H | H | —CH₂F | N |
| 1041 | p-O-i-Pr | H | H | —CH₂F | N |
| 1042 | o-O-n-Bu | H | H | —CH₂F | N |
| 1043 | m-O-n-Bu | H | H | —CH₂F | N |
| 1044 | p-O-n-Bu | H | H | —CH₂F | N |
| 1045 | o-O-i-Bu | H | H | —CH₂F | N |
| 1046 | m-O-i-Bu | H | H | —CH₂F | N |
| 1047 | p-O-i-Bu | H | H | —CH₂F | N |
| 1048 | o-OCF₃ | H | H | —CH₂F | N |
| 1049 | m-OCF₃ | H | H | —CH₂F | N |
| 1050 | pOCF₃ | H | H | —CH₂F | N |
| 1051 | H | CF₃CH₂— | CF₃CH₂— | —CH₂OH | CH |
| 1052 | o-OCH₃ | CF₃CH₂— | CF₃CH₂— | —CH₂OH | CH |
| 1053 | m-OCH₃ | CF₃CH₂— | CF₃CH₂— | —CH₂OH | CH |
| 1054 | p-OCH₃ | CF₃CH₂— | CF₃CH₂— | —CH₂OH | CH |
| 1055 | o-Cl | CF₃CH₂ | CF₃CH₂— | —CH₂OH | CH |
| 1056 | m-Cl | CF₃CH₂— | CF₃CH₂— | —CH₂OH | CH |
| 1057 | p-Cl | CF₃CH₂— | CF₃CH₂— | —CH₂OH | CH |
| 1058 | o-Br | CF₃CH₂— | CF₃CH₂— | —CH₂OH | CH |
| 1059 | m-Br | CF₃CH₂— | CF₃CH₂— | —CH₂OH | CH |
| 1060 | p-Br | CF₃CH₂— | CF₃CH₂— | —CH₂OH | CH |
| 1061 | o-NH₂ | CF₃CH₂ | CF₃CH₂— | —CH₂OH | CH |
| 1062 | m-NH₂ | CF₃CH₂— | CF₃CH₂— | —CH₂OH | CH |
| 1063 | p-NH₂ | CF₃CH₂— | CF₃CH₂— | —CH₂OH | CH |
| 1064 | o-NO₂ | CF₃CH₂— | CF₃CH₂— | —CH₂OH | CH |
| 1065 | m-NO₂ | CF₃CH₂ | CF₃CH₂— | —CH₂OH | CH |
| 1066 | p-NO₂ | CF₃CH₂— | CF₃CH₂— | —CH₂OH | CH |
| 1067 | o-OC₂H₅ | CF₃CH₂— | CF₃CH₂— | —CH₂OH | CH |
| 1068 | m-OC₂H₅ | CF₃CH₂— | CF₃CH₂— | —CH₂OH | CH |
| 1069 | p-OC₂H₅ | CF₃CH₂— | CF₃CH₂— | —CH₂OH | CH |
| 1070 | p-I | CF₃CH₂— | CF₃CH₂— | —CH₂OH | CH |
| 1071 | o-O-n-Pr | CF₃CH₂— | CF₃CH₂— | —CH₂OH | CH |
| 1072 | m-O-n-Pr | CF₃CH₂— | CF₃CH₂— | —CH₂OH | CH |
| 1073 | p-O-n-Pr | CF₃CH₂— | CF₃CH₂— | —CH₂OH | CH |
| 1074 | o-O-i-Pr | CF₃CH₂— | CF₃CH₂ | —CH₂OH | CH |
| 1075 | m-O-i-Pr | CF₃CH₂— | CF₃CH₂— | —CH₂OH | CH |
| 1076 | p-O-i-Pr | CF₃CH₂— | CF₃CH₂— | —CH₂OH | CH |
| 1077 | o-O-n-Bu | CF₃CH₂— | CF₃CH₂— | —CH₂OH | CH |
| 1078 | m-O-n-Bu | CF₃CH₂— | CF₃CH₂— | —CH₂OH | CH |
| 1079 | p-O-n-Bu | CF₃CH₂— | CF₃CH₂— | —CH₂OH | CH |
| 1080 | o-O-i-Bu | CF₃CH₂— | CF₃CH₂— | —CH₂OH | CH |
| 1081 | m-O-i-Bu | CF₃CH₂— | CF₃CH₂— | —CH₂OH | CH |
| 1082 | p-O-i-Bu | CF₃CH₂— | CF₃CH₂— | —CH₂OH | CH |
| 1083 | o-OCF₃ | CF₃CH₂— | CF₃CH₂— | —CH₂OH | CH |
| 1084 | m-OCF₃ | CF₃CH₂— | CF₃CH₂— | —CH₂OH | CH |
| 1085 | p-OCF₃ | CF₃CH₂— | CF₃CH₂— | —CH₂OH | CH |
| 1086 | H | CF₃CH₂— | CF₃CH₂— | —CH₂OH | N |
| 1087 | o-OCH₃ | CF₃CH₂— | CF₃CH₂— | —CH₂OH | N |
| 1088 | m-OCH₃ | CF₃CH₂— | CF₃CH₂— | —CH₂OH | N |
| 1089 | p-OCH₃ | CF₃CH₂— | CF₃CH₂— | —CH₂OH | N |
| 1090 | o-Cl | CF₃CH₂— | CF₃CH₂— | —CH₂OH | N |
| 1091 | m-Cl | CF₃CH₂— | CF₃CH₂— | —CH₂OH | N |
| 1092 | p-Cl | CF₃CH₂— | CF₃CH₂— | —CH₂OH | N |
| 1093 | o-Br | CF₃CH₂— | CF₃CH₂— | —CH₂OH | N |
| 1094 | m-Br | CF₃CH₂— | CF₃CH₂— | —CH₂OH | N |
| 1095 | p-Br | CF₃CH₂— | CF₃CH₂— | —CH₂OH | N |
| 1096 | o-NH₂ | CF₃CH₂— | CF₃CH₂— | —CH₂OH | N |
| 1097 | m-NH₂ | CF₃CH₂— | CF₃CH₂— | —CH₂OH | N |
| 1098 | p-NH₂ | CF₃CH₂— | CF₃CH₂— | —CH₂OH | N |
| 1099 | o-NO₂ | CF₃CH₂— | CF₃CH₂— | —CH₂OH | N |
| 1100 | m-NO₂ | CF₃CH₂— | CF₃CH₂— | —CH₂OH | N |
| 1101 | p-NO₂ | CF₃CH₂— | CF₃CH₂— | —CH₂OH | N |
| 1102 | o-OC₂H₅ | CF₃CH₂— | CF₃CH₂— | —CH₂OH | N |
| 1103 | m-OC₂H₅ | CF₃CH₂— | CF₃CH₂— | —CH₂OH | N |
| 1104 | p-OC₂H₅ | CF₃CH₂— | CF₃CH₂— | —CH₂OH | N |
| 1105 | p-I | CF₃CH₂— | CF₃CH₂— | —CH₂OH | N |
| 1106 | o-O-n-Pr | CF₃CH₂— | CF₃CH₂— | —CH₂OH | N |
| 1107 | m-O-n-Pr | CF₃CH₂— | CF₃CH₂— | —CH₂OH | N |
| 1108 | p-O-n-Pr | CF₃CH₂— | CF₃CH₂— | —CH₂OH | N |
| 1109 | o-O-i-Pr | CF₃CH₂— | CF₃CH₂— | —CH₂OH | N |
| 1110 | m-O-i-Pr | CF₃CH₂— | CF₃CH₂— | —CH₂OH | N |
| 1111 | p-O-i-Pr | CF₃CH₂— | CF₃CH₂— | —CH₂OH | N |
| 1112 | o-O-n-Bu | CF₃CH₂— | CF₃CH₂— | —CH₂OH | N |
| 1113 | m-O-n-Bu | CF₃CH₂— | CF₃CH₂— | —CH₂OH | N |
| 1114 | p-O-n-Bu | CF₃CH₂— | CF₃CH₂— | —CH₂OH | N |
| 1115 | o-O-i-Bu | CF₃CH₂— | CF₃CH₂— | —CH₂OH | N |
| 1116 | m-O-i-Bu | CF₃CH₂— | CF₃CH₂— | —CH₂OH | N |
| 1117 | p-O-i-Bu | CF₃CH₂— | CF₃CH₂— | —CH₂OH | N |
| 1118 | o-OCF₃ | CF₃CH₂— | CF₃CH₂— | —CH₂OH | N |
| 1119 | m-OCF₃ | CF₃CH₂— | CF₃CH₂— | —CH₂OH | N |
| 1120 | p-OCF₃ | CF₃CH₂— | CF₃CH₂— | —CH₂OH | N |
| 1121 | H | CF₃CH₂— | Me— | —CH₂OH | CH |
| 1122 | o-OCH₃ | CF₃CH₂— | Me— | —CH₂OH | CH |
| 1123 | m-OCH₃ | CF₃CH₂— | Me— | —CH₂OH | CH |
| 1124 | p-OCH₃ | CF₃CH₂— | Me— | —CH₂OH | CH |
| 1125 | o-Cl | CF₃CH₂— | Me— | —CH₂OH | CH |
| 1126 | m-Cl | CF₃CH₂— | Me— | —CH₂OH | CH |
| 1127 | p-Cl | CF₃CH₂— | Me— | —CH₂OH | CH |
| 1128 | o-Br | CF₃CH₂— | Me— | —CH₂OH | CH |
| 1129 | m-Br | CF₃CH₂— | Me— | —CH₂OH | CH |
| 1130 | p-Br | CF₃CH₂— | Me— | —CH₂OH | CH |
| 1131 | o-NH₂ | CF₃CH₂— | Me— | —CH₂OH | CH |
| 1132 | m-NH₂ | CF₃CH₂— | Me— | —CH₂OH | CH |
| 1133 | p-NH₂ | CF₃CH₂— | Me— | —CH₂OH | CH |
| 1134 | o-NO₂ | CF₃CH₂— | Me— | —CH₂OH | CH |
| 1135 | m-NO₂ | CF₃CH₂— | Me— | —CH₂OH | CH |
| 1136 | p-NO₂ | CF₃CH₂— | Me— | —CH₂OH | CH |
| 1137 | o-OC₂H₅ | CF₃CH₂— | Me— | —CH₂OH | CH |
| 1138 | m-OC₂H₅ | CF₃CH₂— | Me— | —CH₂OH | CH |
| 1139 | p-OC₂H₅ | CF₃CH₂— | Me— | —CH₂OH | CH |
| 1140 | p-I | CF₃CH₂— | Me— | —CH₂OH | CH |
| 1141 | o-O-n-Pr | CF₃CH₂— | Me— | —CH₂OH | CH |
| 1142 | m-O-n-Pr | CF₃CH₂— | Me— | —CH₂OH | CH |
| 1143 | p-O-n-Pr | CF₃CH₂— | Me— | —CH₂OH | CH |
| 1144 | o-O-i-Pr | CF₃CH₂— | Me— | —CH₂OH | CH |
| 1145 | m-O-i-Pr | CF₃CH₂— | Me— | —CH₂OH | CH |
| 1146 | p-O-i-Pr | CF₃CH₂— | Me— | —CH₂OH | CH |
| 1147 | o-O-n-Bu | CF₃CH₂— | Me— | —CH₂OH | CH |
| 1148 | m-O-n-Bu | CF₃CH₂— | Me— | —CH₂OH | CH |
| 1149 | p-O-n-Bu | CF₃CH₂— | Me— | —CH₂OH | CH |
| 1150 | o-O-i-Bu | CF₃CH₂— | Me— | —CH₂OH | CH |
| 1151 | m-O-i-Bu | CF₃CH₂— | Me— | —CH₂OH | CH |
| 1152 | p-O-i-Bu | CF₃CH₂— | Me— | —CH₂OH | CH |
| 1153 | o-OCF₃ | CF₃CH₂— | Me— | —CH₂OH | CH |
| 1154 | m-OCF₃ | CF₃CH₂— | Me— | —CH₂OH | CH |
| 1155 | p-OCF₃ | CF₃CH₂— | Me— | —CH₂OH | CH |
| 1156 | H | CF₃CH₂— | Me— | —CH₂OH | N |
| 1157 | o-OCH₃ | CF₃CH₂— | Me— | —CH₂OH | N |
| 1158 | m-OCH₃ | CF₃CH₂— | Me— | —CH₂OH | N |
| 1159 | p-OCH₃ | CF₃CH₂— | Me— | —CH₂OH | N |
| 1160 | o-Cl | CF₃CH₂— | Me— | —CH₂OH | N |
| 1161 | m-Cl | CF₃CH₂— | Me— | —CH₂OH | N |
| 1162 | p-Cl | CF₃CH₂— | Me— | —CH₂OH | N |
| 1163 | o-Br | CF₃CH₂— | Me— | —CH₂OH | N |
| 1164 | m-Br | CF₃CH₂— | Me— | —CH₂OH | N |
| 1165 | p-Br | CF₃CH₂— | Me— | —CH₂OH | N |
| 1166 | o-NH₂ | CF₃CH₂— | Me— | —CH₂OH | N |
| 1167 | m-NH₂ | CF₃CH₂— | Me— | —CH₂OH | N |
| 1168 | p-NH₂ | CF₃CH₂— | Me— | —CH₂OH | N |
| 1169 | o-NO₂ | CF₃CH₂— | Me— | —CH₂OH | N |
| 1170 | m-NO₂ | CF₃CH₂— | Me— | —CH₂OH | N |
| 1171 | p-NO₂ | CF₃CH₂— | Me— | —CH₂OH | N |
| 1172 | o-OC₂H₅ | CF₃CH₂— | Me— | —CH₂OH | N |
| 1173 | m-OC₂H₅ | CF₃CH₂— | Me— | —CH₂OH | N |
| 1174 | p-OC₂H₅ | CF₃CH₂— | Me— | —CH₂OH | N |
| 1175 | p-I | CF₃CH₂— | Me— | —CH₂OH | N |
| 1176 | o-O-n-Pr | CF₃CH₂— | Me— | —CH₂OH | N |
| 1177 | m-O-n-Pr | CF₃CH₂— | Me— | —CH₂OH | N |
| 1178 | p-O-n-Pr | CF₃CH₂— | Me— | —CH₂OH | N |
| 1179 | o-O-i-Pr | CF₃CH₂— | Me— | —CH₂OH | N |
| 1180 | m-O-i-Pr | CF₃CH₂— | Me— | —CH₂OH | N |
| 1181 | p-O-i-Pr | CF₃CH₂— | Me— | —CH₂OH | N |
| 1182 | o-O-n-Bu | CF₃CH₂— | Me— | —CH₂OH | N |
| 1183 | m-O-n-Bu | CF₃CH₂— | Me— | —CH₂OH | N |
| 1184 | p-O-n-Bu | CF₃CH₂— | Me— | —CH₂OH | N |
| 1185 | o-O-i-Bu | CF₃CH₂— | Me— | —CH₂OH | N |
| 1186 | m-O-i-Bu | CF₃CH₂— | Me— | —CH₂OH | N |
| 1187 | p-O-i-Bu | CF₃CH₂— | Me— | —CH₂OH | N |
| 1188 | o-OCF₃ | CF₃CH₂— | Me— | —CH₂OH | N |
| 1189 | m-OCF₃ | CF₃CH₂— | Me— | —CH₂OH | N |
| 1190 | p-OCF₃ | CF₃CH₂— | Me— | —CH₂OH | N |
| 1191 | H | CF₃CH₂— | Et— | —CH₂OH | CH |
| 1192 | o-OCH₃ | CF₃CH₂— | Et— | —CH₂OH | CH |
| 1193 | m-OCH₃ | CF₃CH₂— | Et— | —CH₂OH | CH |
| 1194 | p-OCH₃ | CF₃CH₂— | Et— | —CH₂OH | CH |
| 1195 | o-Cl | CF₃CH₂— | Et— | —CH₂OH | CH |
| 1196 | m-Cl | CF₃CH₂— | Et— | —CH₂OH | CH |
| 1197 | p-Cl | CF₃CH₂— | Et— | —CH₂OH | CH |
| 1198 | o-Br | CF₃CH₂— | Et— | —CH₂OH | CH |
| 1199 | m-Br | CF₃CH₂— | Et— | —CH₂OH | CH |
| 1200 | p-Br | CF₃CH₂— | Et— | —CH₂OH | CH |
| 1201 | o-NH₂ | CF₃CH₂— | Et— | —CH₂OH | CH |
| 1202 | m-NH₂ | CF₃CH₂— | Et— | —CH₂OH | CH |
| 1203 | p-NH₂ | CF₃CH₂— | Et— | —CH₂OH | CH |
| 1204 | o-NO₂ | CF₃CH₂— | Et— | —CH₂OH | CH |
| 1205 | m-NO₂ | CF₃CH₂— | Et— | —CH₂OH | CH |
| 1206 | p-NO₂ | CF₃CH₂— | Et— | —CH₂OH | CH |
| 1207 | o-OC₂H₅ | CF₃CH₂— | Et— | —CH₂OH | CH |
| 1208 | m-OC₂H₅ | CF₃CH₂— | Et— | —CH₂OH | CH |
| 1209 | p-OC₂H₅ | CF₃CH₂— | Et— | —CH₂OH | CH |
| 1210 | p-I | CF₃CH₂— | Et— | —CH₂OH | CH |
| 1211 | o-O-n-Pr | CF₃CH₂— | Et— | —CH₂OH | CH |
| 1212 | m-O-n-Pr | CF₃CH₂— | Et— | —CH₂OH | CH |
| 1213 | p-O-n-Pr | CF₃CH₂— | Et— | —CH₂OH | CH |
| 1214 | o-O-i-Pr | CF₃CH₂— | Et— | —CH₂OH | CH |
| 1215 | m-O-i-Pr | CF₃CH₂— | Et— | —CH₂OH | CH |
| 1216 | p-O-i-Pr | CF₃CH₂— | Et— | —CH₂OH | CH |
| 1217 | o-O-n-Bu | CF₃CH₂— | Et— | —CH₂OH | CH |
| 1218 | m-O-n-Bu | CF₃CH₂— | Et— | —CH₂OH | CH |
| 1219 | p-O-n-Bu | CF₃CH₂— | Et— | —CH₂OH | CH |
| 1220 | o-O-i-Bu | CF₃CH₂— | Et— | —CH₂OH | CH |
| 1221 | m-O-i-Bu | CF₃CH₂— | Et— | —CH₂OH | CH |
| 1222 | p-O-i-Bu | CF₃CH₂— | Et— | —CH₂OH | CH |
| 1223 | o-OCF₃ | CF₃CH₂— | Et— | —CH₂OH | CH |
| 1224 | m-OCF₃ | CF₃CH₂— | Et— | —CH₂OH | CH |
| 1225 | p-OCF₃ | CF₃CH₂— | Et— | —CH₂OH | CH |
| 1226 | H | CF₃CH₂— | Et— | —CH₂OH | N |
| 1227 | o-OCH₃ | CF₃CH₂— | Et— | —CH₂OH | N |
| 1228 | m-OCH₃ | CF₃CH₂— | Et— | —CH₂OH | N |
| 1229 | p-OCH₃ | CF₃CH₂— | Et— | —CH₂OH | N |
| 1230 | o-Cl | CF₃CH₂— | Et— | —CH₂OH | N |
| 1231 | m-Cl | CF₃CH₂— | Et— | —CH₂OH | N |
| 1232 | p-Cl | CF₃CH₂— | Et— | —CH₂OH | N |
| 1233 | o-Br | CF₃CH₂— | Et— | —CH₂OH | N |
| 1234 | m-Br | CF₃CH₂— | Et— | —CH₂OH | N |
| 1235 | p-Br | CF₃CH₂— | Et— | —CH₂OH | N |
| 1236 | o-NH₂ | CF₃CH₂— | Et— | —CH₂OH | N |
| 1237 | m-NH₂ | CF₃CH₂— | Et— | —CH₂OH | N |
| 1238 | p-NH₂ | CF₃CH₂— | Et— | —CH₂OH | N |
| 1239 | o-NO₂ | CF₃CH₂— | Et— | —CH₂OH | N |
| 1240 | m-NO₂ | CF₃CH₂— | Et— | —CH₂OH | N |
| 1241 | p-NO₂ | CF₃CH₂— | Et— | —CH₂OH | N |
| 1242 | o-OC₂H₅ | CF₃CH₂— | Et— | —CH₂OH | N |
| 1243 | m-OC₂H₅ | CF₃CH₂— | Et— | —CH₂OH | N |
| 1244 | p-OC₂H₅ | CF₃CH₂— | Et— | —CH₂OH | N |
| 1245 | p-I | CF₃CH₂— | Et— | —CH₂OH | N |
| 1246 | o-O-n-Pr | CF₃CH₂— | Et— | —CH₂OH | N |
| 1247 | m-O-n-Pr | CF₃CH₂— | Et— | —CH₂OH | N |
| 1248 | p-O-n-Pr | CF₃CH₂— | Et— | —CH₂OH | N |
| 1249 | o-O-i-Pr | CF₃CH₂— | Et— | —CH₂OH | N |
| 1250 | m-O-i-Pr | CF₃CH₂— | Et— | —CH₂OH | N |
| 1251 | p-O-i-Pr | CF₃CH₂— | Et— | —CH₂OH | N |
| 1252 | o-O-n-Bu | CF₃CH₂— | Et— | —CH₂OH | N |
| 1253 | m-O-n-Bu | CF₃CH₂— | Et— | —CH₂OH | N |
| 1254 | p-O-n-Bu | CF₃CH₂— | Et— | —CH₂OH | N |
| 1255 | o-O-i-Bu | CF₃CH₂— | Et— | —CH₂OH | N |
| 1256 | m-O-i-Bu | CF₃CH₂— | Et— | —CH₂OH | N |
| 1257 | p-O-i-Bu | CF₃CH₂— | Et— | —CH₂OH | N |
| 1258 | o-OCF₃ | CF₃CH₂— | Et— | —CH₂OH | N |
| 1259 | m-OCF₃ | CF₃CH₂— | Et— | —CH₂OH | N |
| 1260 | p-OCF₃ | CF₃CH₂— | Et— | —CH₂OH | N |
| 1261 | H | CF₃CH₂— | H | —CH₂OH | CH |
| 1262 | o-OCH₃ | CF₃CH₂— | H | —CH₂OH | CH |
| 1263 | m-OCH₃ | CF₃CH₂— | H | —CH₂OH | CH |
| 1264 | p-OCH₃ | CF₃CH₂— | H | —CH₂OH | CH |
| 1265 | o-Cl | CF₃CH₂— | H | —CH₂OH | CH |
| 1266 | m-Cl | CF₃CH₂— | H | —CH₂OH | CH |
| 1267 | p-Cl | CF₃CH₂— | H | —CH₂OH | CH |
| 1268 | o-Br | CF₃CH₂— | H | —CH₂OH | CH |
| 1269 | m-Br | CF₃CH₂— | H | —CH₂OH | CH |
| 1270 | p-Br | CF₃CH₂— | H | —CH₂OH | CH |
| 1271 | o-NH₂ | CF₃CH₂— | H | —CH₂OH | CH |
| 1272 | m-NH₂ | CF₃CH₂— | H | —CH₂OH | CH |
| 1273 | p-NH₂ | CF₃CH₂— | H | —CH₂OH | CH |
| 1274 | o-NO₂ | CF₃CH₂— | H | —CH₂OH | CH |
| 1275 | m-NO₂ | CF₃CH₂— | H | —CH₂OH | CH |
| 1276 | p-NO₂ | CF₃CH₂— | H | —CH₂OH | CH |
| 1277 | o-OC₂H₅ | CF₃CH₂— | H | —CH₂OH | CH |
| 1278 | m-OC₂H₅ | CF₃CH₂— | H | —CH₂OH | CH |
| 1279 | p-OC₂H₅ | CF₃CH₂— | H | —CH₂OH | CH |
| 1280 | p-I | CF₃CH₂— | H | —CH₂OH | CH |
| 1281 | o-O-n-Pr | CF₃CH₂— | H | —CH₂OH | CH |
| 1282 | m-O-n-Pr | CF₃CH₂— | H | —CH₂OH | CH |
| 1283 | p-O-n-Pr | CF₃CH₂— | H | —CH₂OH | CH |
| 1284 | o-O-i-Pr | CF₃CH₂— | H | —CH₂OH | CH |
| 1285 | m-O-i-Pr | CF₃CH₂— | H | —CH₂OH | CH |
| 1286 | p-O-i-Pr | CF₃CH₂— | H | —CH₂OH | CH |
| 1287 | o-O-n-Bu | CF₃CH₂— | H | —CH₂OH | CH |
| 1288 | m-O-n-Bu | CF₃CH₂— | H | —CH₂OH | CH |
| 1289 | p-O-n-Bu | CF₃CH₂— | H | —CH₂OH | CH |
| 1290 | o-O-i-Bu | CF₃CH₂— | H | —CH₂OH | CH |
| 1291 | m-O-i-Bu | CF₃CH₂— | H | —CH₂OH | CH |
| 1292 | p-O-i-Bu | CF₃CH₂— | H | —CH₂OH | CH |
| 1293 | o-OCF₃ | CF₃CH₂— | H | —CH₂OH | CH |
| 1294 | m-OCF₃ | CF₃CH₂— | H | —CH₂OH | CH |
| 1295 | p-OCF₃ | CF₃CH₂— | H | —CH₂OH | CH |
| 1296 | H | CF₃CH₂— | H | —CH₂OH | N |
| 1297 | o-OCH₃ | CF₃CH₂— | H | —CH₂OH | N |
| 1298 | m-OCH₃ | CF₃CH₂— | H | —CH₂OH | N |
| 1299 | p-OCH₃ | CF₃CH₂— | H | —CH₂OH | N |
| 1300 | o-Cl | CF₃CH₂— | H | —CH₂OH | N |
| 1301 | m-Cl | CF₃CH₂— | H | —CH₂OH | N |
| 1302 | p-Cl | CF₃CH₂— | H | —CH₂OH | N |
| 1303 | o-Br | CF₃CH₂— | H | —CH₂OH | N |
| 1304 | m-Br | CF₃CH₂— | H | —CH₂OH | N |
| 1305 | p-Br | CF₃CH₂— | H | —CH₂OH | N |
| 1306 | o-NH₂ | CF₃CH₂— | H | —CH₂OH | N |
| 1307 | m-NH₂ | CF₃CH₂— | H | —CH₂OH | N |
| 1308 | p-NH₂ | CF₃CH₂— | H | —CH₂OH | N |
| 1309 | o-NO₂ | CF₃CH₂— | H | —CH₂OH | N |
| 1310 | m-NO₂ | CF₃CH₂— | H | —CH₂OH | N |
| 1311 | p-NO₂ | CF₃CH₂— | H | —CH₂OH | N |
| 1312 | o-OC₂H₅ | CF₃CH₂— | H | —CH₂OH | N |
| 1313 | m-OC₂H₅ | CF₃CH₂— | H | —CH₂OH | N |
| 1314 | p-OC₂H₅ | CF₃CH₂— | H | —CH₂OH | N |
| 1315 | p-I | CF₃CH₂— | H | —CH₂OH | N |
| 1316 | o-O-n-Pr | CF₃CH₂— | H | —CH₂OH | N |
| 1317 | m-O-n-Pr | CF₃CH₂— | H | —CH₂OH | N |
| 1318 | p-O-n-Pr | CF₃CH₂— | H | —CH₂OH | N |
| 1319 | o-O-i-Pr | CF₃CH₂— | H | —CH₂OH | N |
| 1320 | m-O-i-Pr | CF₃CH₂— | H | —CH₂OH | N |
| 1321 | p-O-i-Pr | CF₃CH₂— | H | —CH₂OH | N |
| 1322 | o-O-n-Bu | CF₃CH₂— | H | —CH₂OH | N |
| 1323 | m-O-n-Bu | CF₃CH₂— | H | —CH₂OH | N |
| 1324 | p-O-n-Bu | CF₃CH₂— | H | —CH₂OH | N |
| 1325 | o-O-i-Bu | CF₃CH₂— | H | —CH₂OH | N |
| 1326 | m-O-i-Bu | CF₃CH₂— | H | —CH₂OH | N |
| 1327 | p-O-i-Bu | CF₃CH₂— | H | —CH₂OH | N |
| 1328 | o-OCF₃ | CF₃CH₂— | H | —CH₂OH | N |
| 1329 | m-OCF₃ | CF₃CH₂— | H | —CH₂OH | N |
| 1330 | p-OCF₃ | CF₃CH₂— | H | —CH₂OH | N |
| 1331 | H | H | H | —CH₂OH | CH |
| 1332 | o-OCH₃ | H | H | —CH₂OH | CH |
| 1333 | m-OCH₃ | H | H | —CH₂OH | CH |
| 1334 | p-OCH₃ | H | H | —CH₂OH | CH |
| 1335 | o-Cl | H | H | - CH₂OH | CH |
| 1336 | m-Cl | H | H | - CH₂OH | CH |
| 1337 | p-Cl | H | H | —CH₂OH | CH |
| 1338 | o-Br | H | H | —CH₂OH | CH |
| 1339 | m-Br | H | H | —CH₂OH | CH |
| 1340 | p-Br | H | H | —CH₂OH | CH |
| 1341 | o-NH₂ | H | H | —CH₂OH | CH |
| 1342 | m-NH₂ | H | H | —CH₂OH | CH |
| 1343 | p-NH₂ | H | H | —CH₂OH | CH |
| 1344 | o-NO₂ | H | H | —CH₂OH | CH |
| 1345 | m-NO₂ | H | H | —CH₂OH | CH |
| 1346 | p-NO₂ | H | H | —CH₂OH | CH |
| 1347 | o-OC₂H₅ | H | H | —CH₂OH | CH |
| 1348 | m-OC₂H₅ | H | H | —CH₂OH | CH |
| 1349 | p-OC₂H₅ | H | H | —CH₂OH | CH |
| 1350 | p-I | H | H | —CH₂OH | CH |
| 1351 | o-O-n-Pr | H | H | —CH₂OH | CH |
| 1352 | m-O-n-Pr | H | H | —CH₂OH | CH |
| 1353 | p-O-n-Pr | H | H | —CH₂OH | CH |
| 1354 | o-O-i-Pr | H | H | —CH₂OH | CH |
| 1355 | m-O-i-Pr | H | H | —CH₂OH | CH |
| 1356 | p-O-i-Pr | H | H | —CH₂OH | CH |
| 1357 | o-O-n-Bu | H | H | —CH₂OH | CH |
| 1358 | m-O-n-Bu | H | H | —CH₂OH | CH |
| 1359 | p-O-n-Bu | H | H | —CH₂OH | CH |
| 1360 | o-O-i-Bu | H | H | —CH₂OH | CH |
| 1361 | m-O-i-Bu | H | H | —CH₂OH | CH |
| 1362 | p-O-i-Bu | H | H | —CH₂OH | CH |
| 1363 | o-OCF₃ | H | H | —CH₂OH | CH |
| 1364 | m-OCF₃ | H | H | —CH₂OH | CH |
| 1365 | p-OCF₃ | H | H | —CH₂OH | CH |
| 1366 | H | H | H | —CH₂OH | N |
| 1367 | o-OCH₃ | H | H | —CH₂OH | N |
| 1368 | m-OCH₃ | H | H | —CH₂OH | N |
| 1369 | p-OCH₃ | H | H | -CH₂OH | N |
| 1370 | o-Cl | H | H | —CH₂OH | N |
| 1371 | m-Cl | H | H | —CH₂OH | N |
| 1372 | p-Cl | H | H | —CH₂OH | N |
| 1373 | o-Br | H | H | —CH₂OH | N |
| 1374 | m-Br | H | H | —CH₂OH | N |
| 1375 | p-Br | H | H | —CH₂OH | N |
| 1376 | o-NH₂ | H | H | —CH₂OH | N |
| 1377 | m-NH₂ | H | H | —CH₂OH | N |
| 1378 | p-NH₂ | H | H | —CH₂OH | N |
| 1379 | o-NO₂ | H | H | —CH₂OH | N |
| 1380 | m-NO₂ | H | H | —CH₂OH | N |
| 1381 | p-NO₂ | H | H | —CH₂OH | N |
| 1382 | o-OC₂H₅ | H | H | —CH₂OH | N |
| 1383 | m-OC₂H₅ | H | H | —CH₂OH | N |
| 1384 | p-OC₂H₅ | H | H | —CH₂OH | N |
| 1385 | p-I | H | H | —CH₂OH | N |
| 1386 | o-O-n-Pr | H | H | —CH₂OH | N |
| 1387 | m-O-n-Pr | H | H | —CH₂OH | N |
| 1388 | p-O-n-Pr | H | H | —CH₂OH | N |
| 1389 | o-O-i-Pr | H | H | —CH₂OH | N |
| 1390 | m-O-i-Pr | H | H | —CH₂OH | N |
| 1391 | p-O-i-Pr | H | H | —CH₂OH | N |
| 1392 | o-O-n-Bu | H | H | —CH₂OH | N |
| 1393 | m-O-n-Bu | H | H | —CH₂OH | N |
| 1394 | p-O-n-Bu | H | H | —CH₂OH | N |
| 1395 | o-O-i-Bu | H | H | —CH₂OH | N |
| 1396 | m-O-i-Bu | H | H | —CH₂OH | N |
| 1397 | p-O-i-Bu | H | H | —CH₂OH | N |
| 1398 | o-OCF₃ | H | H | —CH₂OH | N |
| 1399 | m-OCF₃ | H | H | —CH₂OH | N |
| 1400 | p-OCF₃ | H | H | —CH₂OH | N |
| 1401 | H | —CH₂O-CO-t-Bu | —CH₂O-CO-t-Bu | H | CH |
| 1402 | o-OCH₃ | —CH₂O-CO-t-Bu | —CH₂O-CO-t-Bu | H | CH |
| 1403 | m-OCH₃ | —CH₂O-CO-t-Bu | —CH₂O-CO-t-Bu | H | CH |
| 1404 | p-OCH₃ | —CH₂O-CO-t-Bu | —CH₂O-CO-t-Bu | H | CH |
| 1405 | o-Cl | —CH₂O-CO-t-Bu | —CH₂O-CO-t-Bu | H | CH |
| 1406 | m-Cl | —CH₂O-CO-t-Bu | —CH₂O-CO-t-Bu | H | CH |
| 1407 | p-Cl | —CH₂O-CO-t-Bu | —CH₂O-CO-t-Bu | H | CH |
| 1408 | o-Br | —CH₂O-CO-t-Bu | —CH₂O-CO-t-Bu | H | CH |
| 1409 | m-Br | —CH₂O-CO-t-Bu | —CH₂O-CO-t-Bu | H | CH |
| 1410 | p-Br | —CH₂O-CO-t-Bu | —CH₂O-CO-t-Bu | H | CH |
| 1411 | o-NH₂ | —CH₂O-CO-t-Bu | —CH₂O-CO-t-Bu | H | CH |
| 1412 | m-NH₂ | —CH₂O-CO-t-Bu | —CH₂O-CO-t-Bu | H | CH |
| 1413 | p-NH₂ | —CH₂O-CO-t-Bu | —CH₂O-CO-t-Bu | H | CH |
| 1414 | o-NO₂ | —CH₂O-CO-t-Bu | —CH₂O-CO-t-Bu | H | CH |
| 1415 | m-NO₂ | —CH₂O-CO-t-Bu | —CH₂O-CO-t-Bu | H | CH |
| 1416 | p-NO₂ | —CH₂O-CO-t-Bu | —CH₂O-CO-t-Bu | H | CH |
| 1417 | o-OC₂H₅ | —CH₂O-CO-t-Bu | —CH₂O-CO-t-Bu | H | CH |
| 1418 | m-OC₂H₅ | —CH₂O-CO-t-Bu | —CH₂O-CO-t-Bu | H | CH |
| 1419 | p-OC₂H₅ | —CH₂O-CO-t-Bu | —CH₂O-CO-t-Bu | H | CH |
| 1420 | p-I | —CH₂O-CO-t-Bu | —CH₂O-CO-t-Bu | H | CH |
| 1421 | o-O-n-Pr | —CH₂O-CO-t-Bu | —CH₂O-CO-t-Bu | H | CH |
| 1422 | m-O-n-Pr | —CH₂O-CO-t-Bu | —CH₂O-CO-t-Bu | H | CH |
| 1423 | p-O-n-Pr | —CH₂O-CO-t-Bu | —CH₂O-CO-t-Bu | H | CH |
| 1424 | o-O-i-Pr | —CH₂O-CO-t-Bu | —CH₂O-CO-t-Bu | H | CH |
| 1425 | m-O-i-Pr | —CH₂O-CO-t-Bu | —CH₂O-CO-t-Bu | H | CH |
| 1426 | p-O-i-Pr | —CH₂O-CO-t-Bu | —CH₂O-CO-t-Bu | H | CH |
| 1427 | o-O-n-Bu | —CH₂O-CO-t-Bu | —CH₂O-CO-t-Bu | H | CH |
| 1428 | m-O-n-Bu | —CH₂O-CO-t-Bu | —CH₂O-CO-t-Bu | H | CH |
| 1429 | p-O-n-Bu | —CH₂O-CO-t-Bu | —CH₂O-CO-t-Bu | H | CH |
| 1430 | o-O-i-Bu | —CH₂O-CO-t-Bu | —CH₂O-CO-t-Bu | H | CH |
| 1431 | m-O-i-Bu | —CH₂O-CO-t-Bu | —CH₂O-CO-t-Bu | H | CH |
| 1432 | p-O-i-Bu | —CH₂O-CO-t-Bu | —CH₂O-CO-t-Bu | H | CH |
| 1433 | o-OCF₃ | —CH₂O-CO-t-Bu | —CH₂O-CO-t-Bu | H | CH |
| 1434 | m-OCF₃ | —CH₂O-CO-t-Bu | —CH₂O-CO-t-Bu | H | CH |
| 1435 | p-OCF₃ | —CH₂O-CO-t-Bu | —CH₂O-CO-t-Bu | H | CH |
| 1436 | H | —CH₂O-CO-t-Bu | —CH₂O-CO-t-Bu | Me— | CH |
| 1437 | o-OCH₃ | —CH₂O-CO-t-Bu | —CH₂O-CO-t-Bu | Me— | CH |
| 1438 | m-OCH₃ | —CH₂O-CO-t-Bu | —CH₂O-CO-t-Bu | Me— | CH |
| 1439 | p-OCH₃ | —CH₂O-CO-t-Bu | —CH₂O-CO-t-Bu | Me— | CH |
| 1440 | o-Cl | —CH₂O-CO-t-Bu | —CH₂O-CO-t-Bu | Me— | CH |
| 1441 | m-Cl | —CH₂O-CO-t-Bu | —CH₂O-CO-t-Bu | Me— | CH |
| 1442 | p-Cl | —CH₂O-CO-t-Bu | —CH₂O-CO-t-Bu | Me— | CH |
| 1443 | o-Br | —CH₂O-CO-t-Bu | —CH₂O-CO-t-Bu | Me— | CH |
| 1444 | m-Br | —CH₂O-CO-t-Bu | —CH₂O-CO-t-Bu | Me— | CH |
| 1445 | p-Br | —CH₂O-CO-t-Bu | —CH₂O-CO-t-Bu | Me— | CH |
| 1446 | o-NH₂ | —CH₂O-CO-t-Bu | —CH₂O-CO-t-Bu | Me— | CH |
| 1447 | m-NH₂ | —CH₂O-CO-t-Bu | —CH₂O-CO-t-Bu | Me— | CH |
| 1448 | p-NH₂ | —CH₂O-CO-t-Bu | —CH₂O-CO-t-Bu | Me— | CH |
| 1449 | o-NO₂ | —CH₂O-CO-t-Bu | —CH₂O-CO-t-Bu | Me— | CH |
| 1450 | m-NO₂ | —CH₂O-CO-t-Bu | —CH₂O-CO-t-Bu | Me— | CH |
| 1451 | p-NO₂ | —CH₂O-CO-t-Bu | —CH₂O-CO-t-Bu | Me— | CH |
| 1452 | o-OC₂H₅ | —CH₂O-CO-t-Bu | —CH₂O-CO-t-Bu | Me— | CH |
| 1453 | m-OC₂H₅ | —CH₂O-CO-t-Bu | —CH₂O-CO-t-Bu | Me— | CH |
| 1454 | p-OC₂H₅ | —CH₂O-CO-t-Bu | —CH₂O-CO-t-Bu | Me— | CH |
| 1455 | p-I | —CH₂O-CO-t-Bu | —CH₂O-CO-t-Bu | Me— | CH |
| 1456 | o-O-n-Pr | —CH₂O-CO-t-Bu | —CH₂O-CO-t-Bu | Me— | CH |
| 1457 | m-O-n-Pr | —CH₂O-CO-t-Bu | —CH₂O-CO-t-Bu | Me— | CH |
| 1458 | p-O-n-Pr | —CH₂O-CO-t-Bu | —CH₂O-CO-t-Bu | Me— | CH |
| 1459 | o-O-i-Pr | —CH₂O-CO-t-Bu | —CH₂O-CO-t-Bu | Me— | CH |
| 1460 | m-O-i-Pr | —CH₂O-CO-t-Bu | —CH₂O-CO-t-Bu | Me— | CH |
| 1461 | p-O-i-Pr | —CH₂O-CO-t-Bu | —CH₂O-CO-t-Bu | Me— | CH |
| 1462 | o-O-n-Bu | —CH₂O-CO-t-Bu | —CH₂O-CO-t-Bu | Me— | CH |
| 1463 | m-O-n-Bu | —CH₂O-CO-t-Bu | —CH₂O-CO-t-Bu | Me— | CH |
| 1464 | p-O-n-Bu | —CH₂O-CO-t-Bu | —CH₂O-CO-t-Bu | Me— | CH |
| 1465 | o-O-i-Bu | —CH₂O-CO-t-Bu | —CH₂O-CO-t-Bu | Me— | CH |
| 1466 | m-O-i-Bu | —CH₂O-CO-t-Bu | —CH₂O-CO-t-Bu | Me— | CH |
| 1467 | p-O-i-Bu | —CH₂O-CO-t-Bu | —CH₂O-CO-t-Bu | Me— | CH |
| 1468 | o-OCF₃ | —CH₂O-CO-t-Bu | —CH₂O-CO-t-Bu | Me— | CH |
| 1469 | m-OCF₃ | —CH₂O-CO-t-Bu | —CH₂O-CO-t-Bu | Me— | CH |
| 1470 | p-OCF₃ | —CH₂O-CO-t-Bu | —CH₂O-CO-t-Bu | Me— | CH |
| 1471 | H | —CH₂O-CO-t-Bu | —CH₂O-CO-t-Bu | —CH₂F | CH |
| 1472 | o-OCH₃ | —CH₂O-CO-t-Bu | —CH₂O-CO-t-Bu | —CH₂F | CH |
| 1473 | m-OCH₃ | —CH₂O-CO-t-Bu | —CH₂O-CO-t-Bu | —CH₂F | CH |
| 1474 | p-OCH₃ | —CH₂O-CO-t-Bu | —CH₂O-CO-t-Bu | —CH₂F | CH |
| 1475 | o-Cl | —CH₂O-CO-t-Bu | —CH₂O-CO-t-Bu | —CH₂F | CH |
| 1476 | m-Cl | —CH₂O-CO-t-Bu | —CH₂O-CO-t-Bu | —CH₂F | CH |
| 1477 | p-Cl | —CH₂O-CO-t-Bu | —CH₂O-CO-t-Bu | —CH₂F | CH |
| 1478 | o-Br | —CH₂O-CO-t-Bu | —CH₂O-CO-t-Bu | —CH₂F | CH |
| 1479 | m-Br | —CH₂O-CO-t-Bu | —CH₂O-CO-t-Bu | —CH₂F | CH |
| 1480 | p-Br | —CH₂O-CO-t-Bu | —CH₂O-CO-t-Bu | —CH₂F | CH |
| 1481 | o-NH₂ | —CH₂O-CO-t-Bu | —CH₂O-CO-t-Bu | —CH₂F | CH |
| 1482 | m-NH₂ | —CH₂O-CO-t-Bu | —CH₂O-CO-t-Bu | —CH₂F | CH |
| 1483 | p-NH₂ | —CH₂O-CO-t-Bu | —CH₂O-CO-t-Bu | —CH₂F | CH |
| 1484 | o-NO₂ | —CH₂O-CO-t-Bu | —CH₂O-CO-t-Bu | —CH₂F | CH |
| 1485 | m-NO₂ | —CH₂O-CO-t-Bu | —CH₂O-CO-t-Bu | —CH₂F | CH |
| 1486 | p-NO₂ | —CH₂O-CO-t-Bu | —CH₂O-CO-t-Bu | —CH₂F | CH |
| 1487 | o-OC₂H₅ | —CH₂O-CO-t-Bu | —CH₂O-CO-t-Bu | —CH₂F | CH |
| 1488 | m-OC₂H₅ | —CH₂O-CO-t-Bu | —CH₂O-CO-t-Bu | —CH₂F | CH |
| 1489 | p-OC₂H₅ | —CH₂O-CO-t-Bu | —CH₂O-CO-t-Bu | —CH₂F | CH |
| 1490 | p-I | —CH₂O-CO-t-Bu | —CH₂O-CO-t-Bu | —CH₂F | CH |
| 1491 | o-O-n-Pr | —CH₂O-CO-t-Bu | —CH₂O-CO-t-Bu | —CH₂F | CH |
| 1492 | m-O-n-Pr | —CH₂O-CO-t-Bu | —CH₂O-CO-t-Bu | —CH₂F | CH |
| 1493 | p-O-n-Pr | —CH₂O-CO-t-Bu | —CH₂O-CO-t-Bu | —CH₂F | CH |
| 1494 | o-O-i-Pr | —CH₂O-CO-t-Bu | —CH₂O-CO-t-Bu | —CH₂F | CH |
| 1495 | m-O-i-Pr | —CH₂O-CO-t-Bu | —CH₂O-CO-t-Bu | —CH₂F | CH |
| 1496 | p-O-i-Pr | —CH₂O-CO-t-Bu | —CH₂O-CO-t-Bu | —CH₂F | CH |
| 1497 | o-O-n-Bu | —CH₂O-CO-t-Bu | —CH₂O-CO-t-Bu | —CH₂F | CH |
| 1498 | m-O-n-Bu | —CH₂O-CO-t-Bu | —CH₂O-CO-t-Bu | —CH₂F | CH |
| 1499 | p-O-n-Bu | —CH₂O-CO-t-Bu | —CH₂O-CO-t-Bu | —CH₂F | CH |
| 1500 | o-O-i-Bu | —CH₂O-CO-t-Bu | —CH₂O-CO-t-Bu | —CH₂F | CH |
| 1501 | m-O-i-Bu | —CH₂O-CO-t-Bu | —CH₂O-CO-t-Bu | —CH₂F | CH |
| 1502 | p-O-i-Bu | —CH₂O-CO-t-Bu | —CH₂O-CO-t-Bu | —CH₂F | CH |
| 1503 | o-OCF₃ | —CH₂O-CO-t-Bu | —CH₂O-CO-t-Bu | —CH₂F | CH |
| 1504 | m-OCF₃ | —CH₂O-CO-t-Bu | —CH₂O-CO-t-Bu | —CH₂F | CH |
| 1505 | p-OCF₃ | —CH₂O-CO-t-Bu | —CH₂O-CO-t-Bu | —CH₂F | CH |
| 1506 | H | —CH₂O-CO-t-Bu | —CH₂O-CO-t-Bu | —CH₂OH | CH |
| 1507 | o-OCH₃ | —CH₂O-CO-t-Bu | —CH₂O-CO-t-Bu | —CH₂OH | CH |
| 1508 | m-OCH₃ | —CH₂O-CO-t-Bu | —CH₂O-CO-t-Bu | —CH₂OH | CH |
| 1509 | p-OCH₃ | —CH₂O-CO-t-Bu | —CH₂O-CO-t-Bu | —CH₂OH | CH |
| 1510 | o-Cl | —CH₂O-CO-t-Bu | —CH₂O-CO-t-Bu | —CH₂OH | CH |
| 1511 | m-Cl | —CH₂O-CO-t-Bu | —CH₂O-CO-t-Bu | —CH₂OH | CH |
| 1512 | p-Cl | —CH₂O-CO-t-Bu | —CH₂O-CO-t-Bu | —CH₂OH | CH |
| 1513 | o-Br | —CH₂O-CO-t-Bu | —CH₂O-CO-t-Bu | —CH₂OH | CH |
| 1514 | m-Br | —CH₂O-CO-t-Bu | —CH₂O-CO-t-Bu | —CH₂OH | CH |
| 1515 | p-Br | —CH₂O-CO-t-Bu | —CH₂O-CO-t-Bu | —CH₂OH | CH |
| 1516 | o-NH₂ | —CH₂O-CO-t-Bu | —CH₂O-CO-t-Bu | —CH₂OH | CH |
| 1517 | m-NH₂ | —CH₂O-CO-t-Bu | —CH₂O-CO-t-Bu | —CH₂OH | CH |
| 1518 | p-NH₂ | —CH₂O-CO-t-Bu | —CH₂O-CO-t-Bu | —CH₂OH | CH |
| 1519 | o-NO₂ | —CH₂O-CO-t-Bu | —CH₂O-CO-t-Bu | —CH₂OH | CH |
| 1520 | m-NO₂ | —CH₂O-CO-t-Bu | —CH₂O-CO-t-Bu | —CH₂OH | CH |
| 1521 | p-NO₂ | —CH₂O-CO-t-Bu | —CH₂O-CO-t-Bu | —CH₂OH | CH |
| 1522 | o-OC₂H₅ | —CH₂O-CO-t-Bu | —CH₂O-CO-t-Bu | —CH₂OH | CH |
| 1523 | m-OC₂H₅ | —CH₂O-CO-t-Bu | —CH₂O-CO-t-Bu | —CH₂OH | CH |
| 1524 | p-OC₂H₅ | —CH₂O-CO-t-Bu | —CH₂O-CO-t-Bu | —CH₂OH | CH |
| 1525 | p-I | —CH₂O-CO-t-Bu | —CH₂O-CO-t-Bu | —CH₂OH | CH |
| 1526 | o-O-n-Pr | —CH₂O-CO-t-Bu | —CH₂O-CO-t-Bu | —CH₂OH | CH |
| 1527 | m-O-n-Pr | —CH₂O-CO-t-Bu | —CH₂O-CO-t-Bu | —CH₂OH | CH |
| 1528 | p-O-n-Pr | —CH₂O-CO-t-Bu | —CH₂O-CO-t-Bu | —CH₂OH | CH |
| 1529 | o-O-i-Pr | —CH₂O-CO-t-Bu | —CH₂O-CO-t-Bu | —CH₂OH | CH |
| 1530 | m-O-i-Pr | —CH₂O-CO-t-Bu | —CH₂O-CO-t-Bu | —CH₂OH | CH |
| 1531 | p-O-i-Pr | —CH₂O-CO-t-Bu | —CH₂O-CO-t-Bu | —CH₂OH | CH |
| 1532 | o-O-n-Bu | —CH₂O-CO-t-Bu | —CH₂O-CO-t-Bu | —CH₂OH | CH |
| 1533 | m-O-n-Bu | —CH₂O-CO-t-Bu | —CH₂O-CO-t-Bu | —CH₂OH | CH |
| 1534 | p-O-n-Bu | —CH₂O-CO-t-Bu | —CH₂O-CO-t-Bu | —CH₂OH | CH |
| 1535 | o-O-i-Bu | —CH₂O-CO-t-Bu | —CH₂O-CO-t-Bu | —CH₂OH | CH |
| 1536 | m-O-i-Bu | —CH₂O-CO-t-Bu | —CH₂O-CO-t-Bu | —CH₂OH | CH |
| 1537 | p-O-i-Bu | —CH₂O-CO-t-Bu | —CH₂O-CO-t-Bu | —CH₂OH | CH |
| 1538 | o-OCF₃ | —CH₂O-CO-t-Bu | —CH₂O-CO-t-Bu | —CH₂OH | CH |
| 1539 | m-OCF₃ | —CH₂O-CO-t-Bu | —CH₂O-CO-t-Bu | —CH₂OH | CH |
| 1540 | p-OCF₃ | —CH₂O-CO-t-Bu | —CH₂O-CO-t-Bu | —CH₂OH | CH |
| 1541 | H | —CH₂CH₂S-CO-i-Pr | —CH₂CH₂S-CO-i-Pr | H | CH |
| 1542 | o-OCH₃ | —CH₂CH₂S-CO-i-Pr | —CH₂CH₂S-CO-i-Pr | H | CH |
| 1543 | m-OCH₃ | —CH₂CH₂S-CO-i-Pr | —CH₂CH₂S-CO-i-Pr | H | CH |
| 1544 | p-OCH₃ | —CH₂CH₂S-CO-i-Pr | —CH₂CH₂S-CO-i-Pr | H | CH |
| 1545 | o-Cl | —CH₂CH₂S-CO-i-Pr | —CH₂CH₂S-CO-i-Pr | H | CH |
| 1546 | m-Cl | —CH₂CH₂S-CO-i-Pr | —CH₂CH₂S-CO-i-Pr | H | CH |
| 1547 | p-Cl | —CH₂CH₂S-CO-i-Pr | —CH₂CH₂S-CO-i-Pr | H | CH |
| 1548 | o-Br | —CH₂CH₂S-CO-i-Pr | —CH₂CH₂S-CO-i-Pr | H | CH |
| 1549 | m-Br | —CH₂CH₂S-CO-i-Pr | —CH₂CH₂S-CO-i-Pr | H | CH |
| 1550 | p-Br | —CH₂CH₂S-CO-i-Pr | —CH₂CH₂S-CO-i-Pr | H | CH |
| 1551 | o-NH₂ | —CH₂CH₂S-CO-i-Pr | —CH₂CH₂S-CO-i-Pr | H | CH |
| 1552 | m-NH₂ | —CH₂CH₂S-CO-i-Pr | —CH₂CH₂S-CO-i-Pr | H | CH |
| 1553 | p-NH₂ | —CH₂CH₂S-CO-i-Pr | —CH₂CH₂S-CO-i-Pr | H | CH |
| 1554 | o-NO₂ | —CH₂CH₂S-CO-i-Pr | —CH₂CH₂S-CO-i-Pr | H | CH |
| 1555 | m-NO₂ | —CH₂CH₂S-CO-i-Pr | —CH₂CH₂S-CO-i-Pr | H | CH |
| 1556 | p-NO₂ | —CH₂CH₂S-CO-i-Pr | —CH₂CH₂S-CO-i-Pr | H | CH |
| 1557 | o-OC₂H₅ | —CH₂CH₂S-CO-i-Pr | —CH₂CH₂S-CO-i-Pr | H | CH |
| 1558 | m-OC₂H₅ | —CH₂CH₂S-CO-i-Pr | —CH₂CH₂S-CO-i-Pr | H | CH |
| 1559 | p-OC₂H₅ | —CH₂CH₂S-CO-i-Pr | —CH₂CH₂S-CO-i-Pr | H | CH |
| 1560 | p-I | —CH₂CH₂S-CO-i-Pr | —CH₂CH₂S-CO-i-Pr | H | CH |
| 1561 | o-O-n-Pr | —CH₂CH₂S-CO-i-Pr | —CH₂CH₂S-CO-i-Pr | H | CH |
| 1562 | m-O-n-Pr | —CH₂CH₂S-CO-i-Pr | —CH₂CH₂S-CO-i-Pr | H | CH |
| 1563 | p-O-n-Pr | —CH₂CH₂S-CO-i-Pr | —CH₂CH₂S-CO-i-Pr | H | CH |
| 1564 | o-O-i-Pr | —CH₂CH₂S-CO-i-Pr | —CH₂CH₂S-CO-i-Pr | H | CH |
| 1565 | m-O-i-Pr | —CH₂CH₂S-CO-i-Pr | —CH₂CH₂S-CO-i-Pr | H | CH |
| 1566 | p-O-i-Pr | —CH₂CH₂S-CO-i-Pr | —CH₂CH₂S-CO-i-Pr | H | CH |
| 1567 | o-O-n-Bu | —CH₂CH₂S-CO-i-Pr | —CH₂CH₂S-CO-i-Pr | H | CH |
| 1568 | m-O-n-Bu | —CH₂CH₂S-CO-i-Pr | —CH₂CH₂S-CO-i-Pr | H | CH |
| 1569 | p-O-n-Bu | —CH₂CH₂S-CO-i-Pr | —CH₂CH₂S-CO-i-Pr | H | CH |
| 1570 | o-O-i-Bu | —CH₂CH₂S-CO-i-Pr | —CH₂CH₂S-CO-i-Pr | H | CH |
| 1571 | m-O-i-Bu | —CH₂CH₂S-CO-i-Pr | —CH₂CH₂S-CO-i-Pr | H | CH |
| 1572 | p-O-i-Bu | —CH₂CH₂S-CO-i-Pr | —CH₂CH₂S-CO-i-Pr | H | CH |
| 1573 | o-OCF₃ | —CH₂CH₂S-CO-i-Pr | —CH₂CH₂S-CO-i-Pr | H | CH |
| 1574 | m-OCF₃ | —CH₂CH₂S-CO-i-Pr | —CH₂CH₂S-CO-i-Pr | H | CH |
| 1575 | p-OCF₃ | —CH₂CH₂S-CO-i-Pr | —CH₂CH₂S-CO-i-Pr | H | CH |
| 1576 | H | —CH₂CH₂S-CO-i-Pr | —CH₂CH₂S-CO-i-Pr | Me— | CH |
| 1577 | o-OCH₃ | —CH₂CH₂S-CO-i-Pr | —CH₂CH₂S-CO-i-Pr | Me— | CH |
| 1578 | m-OCH₃ | —CH₂CH₂S-CO-i-Pr | —CH₂CH₂S-CO-i-Pr | Me— | CH |
| 1579 | p-OCH₃ | —CH₂CH₂S-CO-i-Pr | —CH₂CH₂S-CO-i-Pr | Me— | CH |
| 1580 | o-Cl | —CH₂CH₂S-CO-i-Pr | —CH₂CH₂S-CO-i-Pr | Me— | CH |
| 1581 | m-Cl | —CH₂CH₂S-CO-i-Pr | —CH₂CH₂S-CO-i-Pr | Me— | CH |
| 1582 | p-Cl | —CH₂CH₂S-CO-i-Pr | —CH₂CH₂S-CO-i-Pr | Me— | CH |
| 1583 | o-Br | —CH₂CH₂S-CO-i-Pr | —CH₂CH₂S-CO-i-Pr | Me— | CH |
| 1584 | m-Br | —CH₂CH₂S-CO-i-Pr | —CH₂CH₂S-CO-i-Pr | Me— | CH |
| 1585 | p-Br | —CH₂CH₂S-CO-i-Pr | —CH₂CH₂S-CO-i-Pr | Me— | CH |
| 1586 | o-NH₂ | —CH₂CH₂S-CO-i-Pr | —CH₂CH₂S-CO-i-Pr | Me— | CH |
| 1587 | m-NH₂ | —CH₂CH₂S-CO-i-Pr | —CH₂CH₂S-CO-i-Pr | Me— | CH |
| 1588 | p-NH₂ | —CH₂CH₂S-CO-i-Pr | —CH₂CH₂S-CO-i-Pr | Me— | CH |
| 1589 | o-NO₂ | —CH₂CH₂S-CO-i-Pr | —CH₂CH₂S-CO-i-Pr | Me— | CH |
| 1590 | m-NO₂ | —CH₂CH₂S-CO-i-Pr | —CH₂CH₂S-CO-i-Pr | Me— | CH |
| 1591 | p-NO₂ | —CH₂CH₂S-CO-i-Pr | —CH₂CH₂S-CO-i-Pr | Me— | CH |
| 1592 | o-OC₂H₅ | —CH₂CH₂S-CO-i-Pr | —CH₂CH₂S-CO-i-Pr | Me— | CH |
| 1593 | m-OC₂H₅ | —CH₂CH₂S-CO-i-Pr | —CH₂CH₂S-CO-i-Pr | Me— | CH |
| 1594 | p-OC₂H₅ | —CH₂CH₂S-CO-i-Pr | —CH₂CH₂S-CO-i-Pr | Me— | CH |
| 1595 | p-I | —CH₂CH₂S-CO-i-Pr | —CH₂CH₂S-CO-i-Pr | Me— | CH |
| 1596 | o-O-n-Pr | —CH₂CH₂S-CO-i-Pr | —CH₂CH₂S-CO-i-Pr | Me— | CH |
| 1597 | m-O-n-Pr | —CH₂CH₂S-CO-i-Pr | —CH₂CH₂S-CO-i-Pr | Me— | CH |
| 1598 | p-O-n-Pr | —CH₂CH₂S-CO-i-Pr | —CH₂CH₂S-CO-i-Pr | Me— | CH |
| 1599 | o-O-i-Pr | —CH₂CH₂S-CO-i-Pr | —CH₂CH₂S-CO-i-Pr | Me— | CH |
| 1600 | m-O-i-Pr | —CH₂CH₂S-CO-i-Pr | —CH₂CH₂S-CO-i-Pr | Me— | CH |
| 1601 | p-O-i-Pr | —CH₂CH₂S-CO-i-Pr | —CH₂CH₂S-CO-i-Pr | Me— | CH |
| 1602 | o-O-n-Bu | —CH₂CH₂S-CO-i-Pr | —CH₂CH₂S-CO-i-Pr | Me— | CH |
| 1603 | m-O-n-Bu | —CH₂CH₂S-CO-i-Pr | —CH₂CH₂S-CO-i-Pr | Me— | CH |
| 1604 | p-O-n-Bu | —CH₂CH₂S-CO-i-Pr | —CH₂CH₂S-CO-i-Pr | Me— | CH |
| 1605 | o-O-i-Bu | —CH₂CH₂S-CO-i-Pr | —CH₂CH₂S-CO-i-Pr | Me— | CH |
| 1606 | m-O-i-Bu | —CH₂CH₂S-CO-i-Pr | —CH₂CH₂S-CO-i-Pr | Me— | CH |
| 1607 | p-O-i-Bu | —CH₂CH₂S-CO-i-Pr | —CH₂CH₂S-CO-i-Pr | Me— | CH |
| 1608 | o-OCF₃ | —CH₂CH₂S-CO-i-Pr | —CH₂CH₂S-CO-i-Pr | Me— | CH |
| 1609 | m-OCF₃ | —CH₂CH₂S-CO-i-Pr | —CH₂CH₂S-CO-i-Pr | Me— | CH |
| 1610 | p-OCF₃ | —CH₂CH₂S-CO-i-Pr | —CH₂CH₂S-CO-i-Pr | Me— | CH |
| 1611 | H | —CH₂CH₂S-CO-i-Pr | —CH₂CH₂S-CO-i-Pr | —CH₂F | CH |
| 1612 | o-OCH₃ | —CH₂CH₂S-CO-i-Pr | —CH₂CH₂S-CO-i-Pr | —CH₂F | CH |
| 1613 | m-OCH₃ | —CH₂CH₂S-CO-i-Pr | —CH₂CH₂S-CO-i-Pr | —CH₂F | CH |
| 1614 | p-OCH₃ | —CH₂CH₂S-CO-i-Pr | —CH₂CH₂S-CO-i-Pr | —CH₂F | CH |
| 1615 | o-Cl | —CH₂CH₂S-CO-i-Pr | —CH₂CH₂S-CO-i-Pr | —CH₂F | CH |
| 1616 | m-Cl | —CH₂CH₂S-CO-i-Pr | —CH₂CH₂S-CO-i-Pr | —CH₂F | CH |
| 1617 | p-Cl | —CH₂CH₂S-CO-i-Pr | —CH₂CH₂S-CO-i-Pr | —CH₂F | CH |
| 1618 | o-Br | —CH₂CH₂S-CO-i-Pr | —CH₂CH₂S-CO-i-Pr | —CH₂F | CH |
| 1619 | m-Br | —CH₂CH₂S-CO-i-Pr | —CH₂CH₂S-CO-i-Pr | —CH₂F | CH |
| 1620 | p-Br | —CH₂CH₂S-CO-i-Pr | —CH₂CH₂S-CO-i-Pr | —CH₂F | CH |
| 1621 | o-NH₂ | —CH₂CH₂S-CO-i-Pr | —CH₂CH₂S-CO-i-Pr | —CH₂F | CH |
| 1622 | m-NH₂ | —CH₂CH₂S-CO-i-Pr | —CH₂CH₂S-CO-i-Pr | —CH₂F | CH |
| 1623 | p-NH₂ | —CH₂CH₂S-CO-i-Pr | —CH₂CH₂S-CO-i-Pr | —CH₂F | CH |
| 1624 | o-NO₂ | —CH₂CH₂S-CO-i-Pr | —CH₂CH₂S-CO-i-Pr | —CH₂F | CH |
| 1625 | m-NO₂ | —CH₂CH₂S-CO-i-Pr | —CH₂CH₂S-CO-i-Pr | —CH₂F | CH |
| 1626 | p-NO₂ | —CH₂CH₂S-CO-i-Pr | —CH₂CH₂S-CO-i-Pr | —CH₂F | CH |
| 1627 | o-OC₂H₅ | —CH₂CH₂S-CO-i-Pr | —CH₂CH₂S-CO-i-Pr | —CH₂F | CH |
| 1628 | m-OC₂H₅ | —CH₂CH₂S-CO-i-Pr | —CH₂CH₂S-CO-i-Pr | —CH₂F | CH |
| 1629 | p-OC₂H₅ | —CH₂CH₂S-CO-i-Pr | —CH₂CH₂S-CO-i-Pr | —CH₂F | CH |
| 1630 | p-I | —CH₂CH₂S-CO-i-Pr | —CH₂CH₂S-CO-i-Pr | —CH₂F | CH |
| 1631 | o-O-n-Pr | —CH₂CH₂S-CO-i-Pr | —CH₂CH₂S-CO-i-Pr | —CH₂F | CH |
| 1632 | m-O-n-Pr | —CH₂CH₂S-CO-i-Pr | —CH₂CH₂S-CO-i-Pr | —CH₂F | CH |
| 1633 | p-O-n-Pr | —CH₂CH₂S-CO-i-Pr | —CH₂CH₂S-CO-i-Pr | —CH₂F | CH |
| 1634 | o-O-i-Pr | —CH₂CH₂S-CO-i-Pr | —CH₂CH₂S-CO-i-Pr | —CH₂F | CH |
| 1635 | m-O-i-Pr | —CH₂CH₂S-CO-i-Pr | —CH₂CH₂S-CO-i-Pr | —CH₂F | CH |
| 1636 | p-O-i-Pr | —CH₂CH₂S-CO-i-Pr | —CH₂CH₂S-CO-i-Pr | —CH₂F | CH |
| 1637 | o-O-n-Bu | —CH₂CH₂S-CO-i-Pr | —CH₂CH₂S-CO-i-Pr | —CH₂F | CH |
| 1638 | m-O-n-Bu | —CH₂CH₂S-CO-i-Pr | —CH₂CH₂S-CO-i-Pr | —CH₂F | CH |
| 1639 | p-O-n-Bu | —CH₂CH₂S-CO-i-Pr | —CH₂CH₂S-CO-i-Pr | —CH₂F | CH |
| 1640 | o-O-i-Bu | —CH₂CH₂S-CO-i-Pr | —CH₂CH₂S-CO-i-Pr | —CH₂F | CH |
| 1641 | m-O-i-Bu | —CH₂CH₂S-CO-i-Pr | —CH₂CH₂S-CO-i-Pr | —CH₂F | CH |
| 1642 | p-O-i-Bu | —CH₂CH₂S-CO-i-Pr | —CH₂CH₂S-CO-i-Pr | —CH₂F | CH |
| 1643 | o-OCF₃ | —CH₂CH₂S-CO-i-Pr | —CH₂CH₂S-CO-i-Pr | —CH₂F | CH |
| 1644 | m-OCF₃ | —CH₂CH₂S-CO-i-Pr | —CH₂CH₂S-CO-i-Pr | —CH₂F | CH |
| 1645 | p-OCF₃ | —CH₂CH₂S-CO-i-Pr | —CH₂CH₂S-CO-i-Pr | —CH₂F | CH |
| 1646 | H | —CH₂CH₂S-CO-i-Pr | —CH₂CH₂S-CO-i-Pr | —CH₂OH | CH |
| 1647 | o-OCH₃ | —CH₂CH₂S-CO-i-Pr | —CH₂CH₂S-CO-i-Pr | —CH₂OH | CH |
| 1648 | m-OCH₃ | —CH₂CH₂S-CO-i-Pr | —CH₂CH₂S-CO-i-Pr | —CH₂OH | CH |
| 1649 | p-OCH₃ | —CH₂CH₂S-CO-i-Pr | —CH₂CH₂S-CO-i-Pr | —CH₂OH | CH |
| 1650 | o-Cl | —CH₂CH₂S-CO-i-Pr | —CH₂CH₂S-CO-i-Pr | —CH₂OH | CH |
| 1651 | m-Cl | —CH₂CH₂S-CO-i-Pr | —CH₂CH₂S-CO-i-Pr | —CH₂OH | CH |
| 1652 | p-Cl | —CH₂CH₂S-CO-i-Pr | —CH₂CH₂S-CO-i-Pr | —CH₂OH | CH |
| 1653 | o-Br | —CH₂CH₂S-CO-i-Pr | —CH₂CH₂S-CO-i-Pr | —CH₂OH | CH |
| 1654 | m-Br | —CH₂CH₂S-CO-i-Pr | —CH₂CH₂S-CO-i-Pr | —CH₂OH | CH |
| 1655 | p-Br | —CH₂CH₂S-CO-i-Pr | —CH₂CH₂S-CO-i-Pr | —CH₂OH | CH |
| 1656 | o-NH₂ | —CH₂CH₂S-CO-i-Pr | —CH₂CH₂S-CO-i-Pr | —CH₂OH | CH |
| 1657 | m-NH₂ | —CH₂CH₂S-CO-i-Pr | —CH₂CH₂S-CO-i-Pr | —CH₂OH | CH |
| 1658 | p-NH₂ | —CH₂CH₂S-CO-i-Pr | —CH₂CH₂S-CO-i-Pr | —CH₂OH | CH |
| 1659 | o-NO₂ | —CH₂CH₂S-CO-i-Pr | —CH₂CH₂S-CO-i-Pr | —CH₂OH | CH |
| 1660 | m-NO₂ | —CH₂CH₂S-CO-i-Pr | —CH₂CH₂S-CO-i-Pr | —CH₂OH | CH |
| 1661 | p-NO₂ | —CH₂CH₂S-CO-i-Pr | —CH₂CH₂S-CO-i-Pr | —CH₂OH | CH |
| 1662 | o-OC₂H₅ | —CH₂CH₂S-CO-i-Pr | —CH₂CH₂S-CO-i-Pr | —CH₂OH | CH |
| 1663 | m-OC₂H₅ | —CH₂CH₂S-CO-i-Pr | —CH₂CH₂S-CO-i-Pr | —CH₂OH | CH |
| 1664 | p-OC₂H₅ | —CH₂CH₂S-CO-i-Pr | —CH₂CH₂S-CO-i-Pr | —CH₂OH | CH |
| 1665 | p-I | —CH₂CH₂S-CO-i-Pr | —CH₂CH₂S-CO-i-Pr | —CH₂OH | CH |
| 1666 | o-O-n-Pr | —CH₂CH₂S-CO-i-Pr | —CH₂CH₂S-CO-i-Pr | —CH₂OH | CH |
| 1667 | m-O-n-Pr | —CH₂CH₂S-CO-i-Pr | —CH₂CH₂S-CO-i-Pr | —CH₂OH | CH |
| 1668 | p-O-n-Pr | —CH₂CH₂S-CO-i-Pr | —CH₂CH₂S-CO-i-Pr | —CH₂OH | CH |
| 1669 | o-O-i-Pr | —CH₂CH₂S-CO-i-Pr | —CH₂CH₂S-CO-i-Pr | —CH₂OH | CH |
| 1670 | m-O-i-Pr | —CH₂CH₂S-CO-i-Pr | —CH₂CH₂S-CO-i-Pr | —CH₂OH | CH |
| 1671 | p-O-i-Pr | —CH₂CH₂S-CO-i-Pr | —CH₂CH₂S-CO-i-Pr | —CH₂OH | CH |
| 1672 | o-O-n-Bu | —CH₂CH₂S-CO-i-Pr | —CH₂CH₂S-CO-i-Pr | —CH₂OH | CH |
| 1673 | m-O-n-Bu | —CH₂CH₂S-CO-i-Pr | —CH₂CH₂S-CO-i-Pr | —CH₂OH | CH |
| 1674 | p-O-n-Bu | —CH₂CH₂S-CO-i-Pr | —CH₂CH₂S-CO-i-Pr | —CH₂OH | CH |
| 1675 | o-O-i-Bu | —CH₂CH₂S-CO-i-Pr | —CH₂CH₂S-CO-i-Pr | —CH₂OH | CH |
| 1676 | m-O-i-Bu | —CH₂CH₂S-CO-i-Pr | —CH₂CH₂S-CO-i-Pr | —CH₂OH | CH |
| 1677 | p-O-i-Bu | —CH₂CH₂S-CO-i-Pr | —CH₂CH₂S-CO-i-Pr | —CH₂OH | CH |
| 1678 | o-OCF₃ | —CH₂CH₂S-CO-i-Pr | —CH₂CH₂S-CO-i-Pr | —CH₂OH | CH |
| 1679 | m-OCF₃ | —CH₂CH₂S-CO-i-Pr | —CH₂CH₂S-CO-i-Pr | —CH₂OH | CH |
| 1680 | p-OCF₃ | —CH₂CH₂S-CO-i-Pr | —CH₂CH₂S-CO-i-Pr | —CH₂OH | CH |

TABLE 1-b

| No. | R¹ | R² | R³ | R⁴ | X |
|---|---|---|---|---|---|
| 1 | 2-OH | CF₃CH₂— | CF₃CH₂— | H | CH |
| 2 | 3-OH | CF₃CH₂— | CF₃CH₂— | H | CH |
| 3 | 4-OH | CF₃CH₂— | CF₃CH₂— | H | CH |
| 4 | 2-OH | CF₃CH₂— | CF₃CH₂— | H | N |
| 5 | 3-OH | CF₃CH₂— | CF₃CH₂— | H | N |
| 6 | 4-OH | CF₃CH₂— | CF₃CH₂— | H | N |
| 7 | 2-OH | CF₃CH₂— | Me— | H | CH |
| 8 | 3-OH | CF₃CH₂— | Me— | H | CH |
| 9 | 4-OH | CF₃CH₂— | Me— | H | CH |
| 10 | 2-OH | CF₃CH₂— | Me— | H | N |
| 11 | 3-OH | CF₃CH₂— | Me— | H | N |
| 12 | 4-OH | CF₃CH₂— | Me— | H | N |
| 13 | 2-OH | CF₃CH₂— | Et— | H | CH |
| 14 | 3-OH | CF₃CH₂— | Et— | H | CH |
| 15 | 4-OH | CF₃CH₂— | Et— | H | CH |
| 16 | 2-OH | CF₃CH₂— | H | H | CH |
| 17 | 3-OH | CF₃CH₂— | H | H | CH |
| 18 | 4-OH | CF₃CH₂— | H | H | CH |
| 19 | 2-OH | CF₃CH₂— | H | H | N |
| 20 | 3-OH | CF₃CH₂— | H | H | N |
| 21 | 4-OH | CF₃CH₂— | H | H | N |
| 22 | 2-OH | H | H | H | CH |
| 23 | 3-OH | H | H | H | CH |
| 24 | 4-OH | H | H | H | CH |
| 25 | 2-OH | H | H | H | N |
| 26 | 3-OH | H | H | H | N |
| 27 | 4-OH | H | H | H | N |
| 28 | 2-OH | CF₃CH₂— | CF₃CH₂— | Me— | CH |
| 29 | 3-OH | CF₃CH₂— | CF₃CH₂— | Me— | CH |
| 30 | 4-OH | CF₃CH₂— | CF₃CH₂— | Me— | CH |
| 31 | 2-OH | CF₃CH₂— | CF₃CH₂— | Me— | N |
| 32 | 3-OH | CF₃CH₂— | CF₃CH₂— | Me— | N |
| 33 | 4-OH | CF₃CH₂— | CF₃CH₂— | Me— | N |
| 34 | 2-OH | CF₃CH₂— | Me— | Me— | CH |
| 35 | 3-OH | CF₃CH₂— | Me— | Me— | CH |
| 36 | 4-OH | CF₃CH₂— | Me— | Me— | CH |
| 37 | 2-OH | CF₃CH₂— | Me— | Me— | N |
| 38 | 3-OH | CF₃CH₂— | Me— | Me— | N |
| 39 | 4-OH | CF₃CH₂— | Me— | Me— | N |
| 40 | 2-OH | CF₃CH₂— | Et— | Me— | CH |
| 41 | 3-OH | CF₃CH₂— | Et— | Me— | CH |
| 42 | 4-OH | CF₃CH₂— | Et— | Me— | CH |
| 43 | 2-OH | CF₃CH₂— | Et— | Me— | N |
| 44 | 3-OH | CF₃CH₂— | Et— | Me— | N |
| 45 | 4-OH | CF₃CH₂— | Et— | Me— | N |
| 46 | 2-OH | CF₃CH₂— | H | Me— | CH |
| 47 | 3-OH | CF₃CH₂— | H | Me— | CH |
| 48 | 4-OH | CF₃CH₂— | H | Me— | CH |
| 49 | 2-OH | CF₃CH₂— | H | Me— | N |
| 50 | 3-OH | CF₃CH₂— | H | Me— | N |
| 51 | 4-OH | CF₃CH₂— | H | Me— | N |
| 52 | 2-OH | H | H | Me— | CH |
| 53 | 3-OH | H | H | Me— | CH |
| 54 | 4-OH | H | H | Me— | CH |
| 55 | 2-OH | H | H | Me— | N |
| 56 | 3-OH | H | H | Me— | N |
| 57 | 4-OH | H | H | Me— | N |
| 58 | 2-OH | —CH₂O-CO-t-Bu | —CH₂O-CO-t-Bu | H | CH |
| 59 | 3-OH | —CH₂O-CO-t-Bu | —CH₂O-CO-t-Bu | H | CH |
| 60 | 4-OH | —CH₂O-CO-t-Bu | —CH₂O-CO-t-Bu | H | CH |
| 61 | 2-OH | —CH₂O-CO-t-Bu | —CH₂O-CO-t-Bu | Me— | CH |
| 62 | 3-OH | —CH₂O-CO-t-Bu | —CH₂O-CO-t-Bu | Me— | CH |
| 63 | 4-OH | —CH₂O-CO-t-Bu | —CH₂O-CO-t-Bu | Me— | CH |
| 64 | 2-OH | —CH₂CH₂S-CO-i-Pr | —CH₂CH₂S-CO-i-Pr | H | CH |
| 65 | 3-OH | —CH₂CH₂S-CO-i-Pr | —CH₂CH₂S-CO-i-Pr | H | CH |
| 66 | 4-OH | —CH₂CH₂S-CO-i-Pr | —CH₂CH₂S-CO-i-Pr | H | CH |
| 67 | 2-OH | —CH₂CH₂S-CO-i-Pr | —CH₂CH₂S-CO-i-Pr | Me— | CH |
| 68 | 3-OH | —CH₂CH₂S-CO-i-Pr | —CH₂CH₂S-CO-i-Pr | Me— | CH |
| 69 | 4-OH | —CH₂CH₂S-CO-i-Pr | —CH₂CH₂S-CO-i-Pr | Me— | CH |

The compounds used in the present invention are useful as an active ingredient for a pharmaceutical, and specifically are useful against a YMDD-mutation bearing virus, as shown in the test examples mentioned below. A target virus to which the pharmaceutical agent of the present invention is applicable is not particularly limited. Specifically, RNA virus such as human immunodeficiency virus and a DNA virus such as hepatitis B virus are exemplified, and more preferably, hepatitis B virus is exemplified.

Where the compound used in the present invention is used as a pharmaceutical agent, it may be administered alone, and it is preferred that, using a pharmaceutically acceptable additive, a pharmaceutical composition comprising the above compound as an active ingredient is prepared and administered. The composition of the pharmaceutical composition is determined by the solubility of the compound, chemical properties, administration route, dosage regimen and the like. For example, the compound of the present invention can be orally administered, taking the dosage form of a granule, a parvule, a powder, a tablet, a hard syrup, a soft capsule, a troche, a syrup, an emulsion, a soft gelatine capsule, a gel, a paste, a suspension, a liposome and the like, or the compound can be administered intravenously, intramuscularly or subcutaneously as the form of an injection. Also, powders for injection can be prepared from the compound of the present invention so that a parenteral solution can be prepared before using.

As a pharmaceutically acceptable additive, an organic or inorganic, solid or liquid carrier, which is suitable for an oral, enteral, parenteral or local administration, can be used. Examples of a solid carrier used for the preparation of a solid formulation include lactose, sucrose, starch, talc, cellulose, dextrin, kaoline, calcium carbonate, agar, pectin, stearic acid, magnesium stearate, lecithin, sodium chloride and the like. Examples of a liquid carrier used for the preparation of a liquid formulation for oral administration include glycerine, peanut oil, polyvinylpyrrolidone, olive oil, ethanol, benzyl alcohol, propylene glycol, physiological saline, water and the like. The above pharmaceutical composition can also comprise, in addition to the above carriers, an auxiliary agent such as a wetting agent, a suspension aid, a sweetener, a flavor, a coloring agent and a preservative. Further, for the use as a liquid agent, it may be contained in a capsule of a substance which can be absorbed such as gelatin. Examples of a solvent or a suspending agent which is used for the preparation of a formulation for parenteral administration such as an injection, include water, propylene glycol, polyethylene glycol, benzyl alcohol, ethyl oleate, lecithin and the like.

Considering the properties of the known compounds, it can easily be assumed that the compound of the present invention has a high oral absorbency. Therefore, oral administration is a preferred administration route for the pharmaceutical agent of the present invention. The preparation of each of the above agents can be carried out according to standard techniques. Where the agent of the present invention is used for oral administration, the clinical dose is usually 0.1 to 500 mg of the compound per kg adult per day, and preferably 1 to 50 mg of the compound per kg adult per day. This dose may be changed as appropriate, depending on age, disease condition, symptom, the presence or absence of concurrent administration and the like. The above dose may be applied once a day or divided over two to several administrations per day at regular intervals, or may also be applied intermittently every several days. Where the compound of the present invention is used as an injection, the applied dose is 0.01 to 50 mg of the compound per kg adult per day, being preferably 0.1 to 5 mg per kg.

### EXAMPLES

The present invention is further described in the following example. The scope of the present invention is not limited the example. The compound number in the example corresponds to that in Table 1.

### Example 1

Inhibitory Effect against the Replication of YMDD Mutant HBV

The inhibitory effect of the compound of the present invention against the replication of a YMDD mutant HBV which is lamivudine-resistant was measured by a known method (Ono, S. K., et al., J. Clin. Invest. 107, 449-455 (2001)). That is, HuH-7 cell line (derived from human hepatoma) was cultured at 37° C. in a 10% fetal bovine serum-containing Dulbecco's MEM medium on a 60 mm culture dish until the cells were extended to 80 to 90% of the area. 0.9 μg of full length DNA of a wild type HBV or a YMDD mutant HBV was transfected into the cells. After 24 hours of culture, the medium was replaced by a medium containing 1 to 10 μM at final concentration of the test compound or a medium which does not contain the test compound (negative control). After 3 days of culture, the cells were collected. The transfection efficiency was confirmed by concurrently transfecting 0.1 μg of plasmid expressing a β-galactosidase gene into the cells and measuring the β-galactosidase activity. To measure the growth level of HBV, the core particle of the virus was prepared from the cells by a known method (Gunthers, S., et al., J. Virol. 69, 5437-5444 (1995)) and treated at 37° C. for 30 minutes with 10 mM MgCl₂ and 0.1 mg/ml DNase I (final concentrations). By adding ethylenediaminetetraacetic acid at a final concentration of 25 mM, the reaction was terminated, and the mixture was treated at 50° C. for 4 hours with 1% sodium dodecyl sulfate and 0.5 mg/ml proteinase K. After the obtained solution was treated with phenol/chloroform, nucleic acid was precipitated with ethanol. HBV-DNA contained in this sample was detected by Southern blotting. The value obtained by standardizing the amount of single-stranded HBV-DNA in the sample by a β-galactosidase acitivity was defined as a measurement value. Then, the percentage of each measurement value based on a negative control value was determined in each experiment.

The total 3 types of YMDD mutant HBVs were prepared: a HBV having a codon substituting isoleucine for methionine in YMDD motif (M552I), a HBV having a codon substituting valine for methionine (M552V), and a HBV having both of B domain mutations L528M and M552V (L528M/M552V).

Table 2 shows the mean percent control value based on a negative control and its standard deviation, which were obtained by performing the similar experiment in triplicate or quadruplicate for each mutant virus. The number (N) of experiments, which was a base of the average value and the calculation of standard deviation, was shown in a parenthesis. The values in the case of lamivudine were given with reference to a publication (Ono, S. K., et al., J. Clin. Invest. 107, 449-455 (2001)).

TABLE 2

| Test | | | | |
|---|---|---|---|---|
| compound | | | | L528M/ |
| No. 3 | Wild type | M5521 | M552V | M552V |
| 1 μM | 15.3 ± 11.7 | 108.9 ± 5.4 | 62.1 ± 8.3 | 60.9 ± 8.5 |
| | (N = 3) | (N = 2) | (N = 3) | (N = 4) |
| 3 μM | 5.7 ± 4.0 | 36.4 ± 18.1 | 59.7 ± 13.8 | 39.1 ± 8.9 |
| | (N = 3) | (N = 2) | (N = 3) | (N = 4) |
| 5 μM | not determined | 18.5 ± 4.0 | 54.8 ± 21.9 | 29.0 ± 7.8 |
| | | (N = 2) | (N = 2) | (N = 2) |
| 8.7 μM | 2.2 ± 1.6 | 26.0 | 15.7 | 6.0 ± 4.8 |
| | (N = 3) | (N = 1) | (N = 1) | (N = 2) |
| 10 μM | 2.1 ± 2.3 | 13.1 ± 9.5 | 32.1 ± 10.4 | 9.1 ± 6.9 |
| | (N = 2) | (N = 3) | (N = 3) | (N = 4) |
| lamivudine | 1.3 ± 0.2 | 176.8 ± 17.9 | 76.9 ± 15.8 | 126.7 ± |
| 10 μM | | | | 9.6 |

It has been reported that, while lamivudine at a final concentration of 10 μM inhibits almost completely the growth of a wild type HBV, since the drug sensitivity of a YMDD mutant HBV is significantly decreased, the use of the lamivudine, in some cases, may promote the growth of the mutant HBV. As against this lamivudine, test compound No. 3 did not only inhibit the growth of a wild type HBV and a single YMDD mutation bearing HBV, but also inhibited the growth of a YMDD mutant HBV with mutation L528 M in a dose-dependent manner in a dose of 1 to 10 μM of the compound in this experiment.

Industrial Applicability

The phosphonate nucleotide compound used in the present invention which is effective against a YMDD mutant virus has an excellent antiviral activity and a high oral absorbency, and has no toxicity such as bone marrow cell growth inhibition or mutagenicity. Therefore, the present invention can provide an antiviral agent which is important to treat hepatitis B and human immunodeficiency syndrome.

The present disclosure relates to subject matter contained in priority Korean Patent Application No. 10-2001-0035172, filed on Jun. 20, 2001, the contents of which is herein expressly incorporated by reference in its entirety.

1. A method for treating a drug resistant virus disease, which comprises administering a pharmacologically effective amount of a phosphonate nucleotide compound represented by the following formula (I): 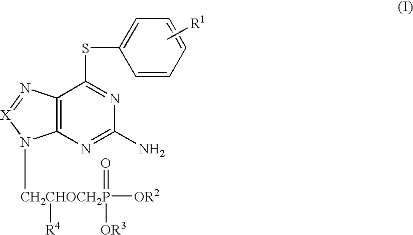
wherein
R¹ is a hydrogen atom, a C₁-C₆ alkoxy group, a C₁-C₄ alkoxy group substituted by one or more halogen atoms, a halogen atom, an amino group, a hydroxyl group or a nitro group;
each of R² and R³ is independently a hydrogen atom, a C₁-C₂₂ alkyl group, an acyloxymethyl group, an acylthioethyl group or an ethyl group substituted by one or more halogen atoms;
R⁴ is a hydrogen atom, a C₁-C₄ alkyl group, a C₁-C₄ hydroxyalkyl group or a C₁-C₄ alkyl group substituted by one ore more halogen atoms; and
X is CH or a nitrogen atom,
or a salt thereof, or a hydrate thereof or a solvate thereof.

2. The method according to claim 1, wherein R¹ is a hydrogen atom, a C₁-C₆ alkoxy group, a C₁-C₄ alkoxy group substituted by one or more halogen atoms, a halogen atom, an amino group, a hydroxyl group or a nitro group; and
each of R² and R³ is independently a hydrogen atom, a C₁-C₂₂ alkyl group or an ethyl group substituted by one or more halogen atoms.

3. The method according to claim 1, wherein R¹ is a hydrogen atom, a C₁-C₆ alkoxy group, a C₁-C₄ alkoxy group substituted by one or more halogen atoms, a halogen atom, an amino group, a hydroxyl group or a nitro group;
each of R² and R³ is independently a hydrogen atom, a C₁-C₂₂ alkyl group or a 2,2,2-trifluoroethyl group; and
R⁴ is a hydrogen atom or a methyl group.

4. The method according to claim 1,
wherein R¹ is a hydrogen atom, a C₁-C₆ alkoxy group, a C₁-C₄ alkoxy group substituted by one or more halogen atoms, a halogen atom, an amino group, a hydroxyl group or a nitro group;
each of R² and R³ is inpendently a hydrogen atom or a 2,2,2-trifluoroethyl group; and
R⁴ is a hydrogen atom or a methyl group.

5. The method according to claim 4, wherein the phosphonate nucleotide compound is selected from the group consisting of the following compounds:
2-amino-9-[2-[bis(2,2,2-trifluoroethyl)phosphonylmethoxy]ethyl]-6-phenylthiopurine;
2-amino-9-[2-[bis(2,2,2-trifluoroethyl)phosphonylmethoxy]ethyl]-6-p-methoxyphenylth iopurine;
2-amino-9-[2-[bis(2,2,2-trifluoroethyl)phosphonylmethoxy]ethyl]-6-m-methoxyphenylt hiopurine;
2-amino-9-[2-[bis(2,2,2-trifluoroethyl)phosphonylmethoxy]ethyl]-6-o-methoxyphenylth iopurine;
2-amino-9-[2-[bis(2,2,2-trifluoroethyl)phosphonylmethoxy]ethyl]-6-p-ethoxyphenylthio purine;
2-amino-9-[2-[bis(2,2,2-trifluoroethyl)phosphonylmethoxy]propyl]-6-phenylthiopurine;
2-amino-9-[2-[bis(2,2,2-trifluoroethyl)phosphonylmethoxy]propyl]-6-p-methoxyphenylthiopurine;
2-amino-9-[2-[bis(2,2,2-trifluoroethyl)phosphonylmethoxy]ethyl]-6-p-butoxyphenylthiopurine;
2-amino-9-[2-[bis(2,2,2-trifluoroethyl)phosphonylmethoxy]ethyl]-6-p-propoxyphenylthiopurine;
2-amino-9-[2-[bis(2,2,2-trifluoroethyl)phosphonylmethoxy]ethyl]-6-p-isopropoxyphenylthiopurine;
2-amino-9-[2-[bis(2,2,2-trifluoroethyl)phosphonylmethoxy]ethyl]-6-p-isobutoxyphenylthiopurine;
2-amino-9-[2-[bis(2,2,2-trifluoroethyl)phosphonylmethoxy]ethyl]-6-p-hydroxyphenylthiopurine;
2-amino-9-[2-[bis(2,2,2-trifluoroethyl)phosphonylmethoxy]ethyl]-6-m-hydroxyphenylthiopurine;
2-amino-9-[2-[bis(2,2,2-trifluoroethyl)phosphonylmethoxy]ethyl]-6-o-hydroxyphenylthiopurine;
2-amino-9-[2-[bis(2,2,2-trifluoroethyl)phosphonylmethoxy]propyl]-6-p-hydroxyphenylthiopurine;
2-amino-9-[2-[bis(2,2,2-trifluoroethyl)phosphonylmethoxy]propyl]-6-m-hydroxyphenylthiopurine;
2-amino-9-[2-[bis(2,2,2-trifluoroethyl)phosphonylmethoxy]propyl]-6-o-hydroxyphenylthiopurine;
2-amino-9-[2-[bis(2,2,2-trifluoroethyl)phosphonylmethoxy]ethyl]-6-m-ethoxyphenylthiopurine;
2-amino-9-[2-[bis(2,2,2-trifluoroethyl)phosphonylmethoxy]ethyl]-6-o-ethoxyphenylthiopurine;
2-amino-9-[2-[bis(2,2,2-trifluoroethyl)phosphonylmethoxy]ethyl]-6-m-butoxyphenylthiopurine;
2-amino-9-[2-[bis(2,2,2-trifluoroethyl)phosphonylmethoxy]ethyl]-6-m-propoxyphenylthiopurine;
2-amino-9-[2-[bis(2,2,2-trifluoroethyl)phosphonylmethoxy]ethyl]-6-m-isopropoxyphenylthiopurine;
2-amino-9-[2-[bis(2,2,2-trifluoroethyl)phosphonylmethoxy]ethyl]-6-m-isobutoxyphenylthiopurine;
2-amino-9-[2-[bis(2,2,2-trifluoroethyl)phosphonylmethoxy]ethyl]-6-o-butoxyphenylthiopurine;
2-amino-9-[2-[bis(2,2,2-trifluoroethyl)phosphonylmethoxy]ethyl]-6-o-propoxyphenylthiopurine;
2-amino-9-[2-[bis(2,2,2-trifluoroethyl)phosphonylmethoxy]ethyl]-6-o-isopropoxyphenylthiopurine;
2-amino-9-[2-[bis(2,2,2-trifluoroethyl)phosphonylmethoxy]ethyl]-6-o-isobutoxyphenylthiopurine;
2-amino-9-[2-[bis(2,2,2-trifluoroethyl)phosphonylmethoxy]propyl]-6-m-methoxyphenylthiopurine;
2-amino-9-[2-[bis(2,2,2-trifluoroethyl)phosphonylmethoxy]propyl]-6-o-methoxyphenylthiopurine;
2-amino-9-[2-[bis(2,2,2-trifluoroethyl)phosphonylmethoxy]propyl]-6-p-ethoxyphenylthiopurine;
2-amino-9-[2-[bis(2,2,2-trifluoroethyl)phosphonylmethoxy]propyl]-6-m-ethoxyphenylthiopurine;
2-amino-9-[2-[bis(2,2,2-trifluoroethyl)phosphonylmethoxy]propyl]-6-o-ethoxyphenylthiopurine;
2-amino-9-[2-[bis(2,2,2-trifluoroethyl)phosphonylmethoxy]propyl]-6-p-butoxyphenylthiopurine;
2-amino-9-[2-[bis(2,2,2-trifluoroethyl)phosphonylmethoxy]propyl]-6-p-propoxyphenylthiopurine;
2-amino-9-[2-[bis(2,2,2-trifluoroethyl)phosphonylmethoxy]propyl]-6-p-isopropoxyphenylthiopurine;
2-amino-9-[2-[bis(2,2,2-trifluoroethyl)phosphonylmethoxy]propyl]-6-p-isobutoxyphenylthiopurine;
2-amino-9-[2-[bis(2,2,2-trifluoroethyl)phosphonylmethoxy]propyl]-6-m-butoxyphenylthiopurine;
2-amino-9-[2-[bis(2,2,2-trifluoroethyl)phosphonylmethoxy]propyl]-6-m-propoxyphenylthiopurine;
2-amino-9-[2-[bis(2,2,2-trifluoroethyl)phosphonylmethoxy]propyl]-6-m-isopropoxyphenylthiopurine;
2-amino-9-[2-[bis(2,2,2-trifluoroethyl)phosphonylmethoxy]propyl]-6-m-isobutoxyphenylthiopurine;
2-amino-9-[2-[bis(2,2,2-trifluoroethyl)phosphonylmethoxy]propyl]-6-o-butoxyphenylthiopurine;
2-amino-9-[2-[bis(2,2,2-trifluoroethyl)phosphonylmethoxy]propyl]-6-o-propoxyphenylthiopurine;
2-amino-9-[2-[bis(2,2,2-trifluoroethyl)phosphonylmethoxy]propyl]-6-o-isopropoxyphenylthiopurine;
2-amino-9-[2-[bis(2,2,2-trifluoroethyl)phosphonylmethoxy]propyl]-6-o-isobutoxyphenylthiopurine;
2-amino-9-[2-[(2,2,2-trifluoroethyl)phosphonylmethoxy]ethyl]-6-phenylthiopurine;
2-amino-9-[2-[(2,2,2-trifluoroethyl)phosphonylmethoxy]ethyl]-6-p-methoxyphenylthiopurine;
2-amino-9-[2-[(2,2,2-trifluoroethyl)phosphonylmethoxy]ethyl]-6-m-methoxyphenylthiopurine;
2-amino-9-[2-[(2,2,2-trifluoroethyl)phosphonylmethoxy]ethyl]-6-o-methoxyphenylthiopurine;
2-amino-9-[2-[(2,2,2-trifluoroethyl)phosphonylmethoxy]ethyl]-6-p-ethoxyphenylthiopurine;
2-amino-9-[2-[(2,2,2-trifluoroethyl)phosphonylmethoxy]ethyl]-6-m-ethoxyphenylthiopurine;
2-amino-9-[2-[(2,2,2-trifluoroethyl)phosphonylmethoxy]ethyl]-6-o-ethoxyphenylthiopurine;
2-amino-9-[2-[(2,2,2-trifluoroethyl)phosphonylmethoxy]ethyl]-6-p-butoxyphenylthiopurine;
2-amino-9-[2-[(2,2,2-trifluoroethyl)phosphonylmethoxy]ethyl]-6-p-propoxyphenylthiopurine;
2-amino-9-[2-[(2,2,2-trifluoroethyl)phosphonylmethoxy]ethyl]-6-p-isopropoxyphenylthiopurine;
2-amino-9-[2-[(2,2,2-trifluoroethyl)phosphonylmethoxy]ethyl]-6-p-isobutoxyphenylthiopurine;
2-amino-9-[2-[(2,2,2-trifluoroethyl)phosphonylmethoxy]ethyl]-6-m-butoxyphenylthiopurine;
2-amino-9-[2-[(2,2,2-trifluoroethyl)phosphonylmethoxy]ethyl]-6-m-propoxyphenylthiopurine;
2-amino-9-[2-[(2,2,2-trifluoroethyl)phosphonylmethoxy]ethyl]-6-m-isopropoxyphenylthiopurine;
2-amino-9-[2-[(2,2,2-trifluoroethyl)phosphonylmethoxy]ethyl]-6-m-isobutoxyphenylthiopurine;
2-amino-9-[2-[(2,2,2-trifluoroethyl)phosphonylmethoxy]ethyl]-6-o-butoxyphenylthiopurine;
2-amino-9-[2-[(2,2,2-trifluoroethyl)phosphonylmethoxy]ethyl]-6-o-propoxyphenylthiopurine;
2-amino-9-[2-[(2,2,2-trifluoroethyl)phosphonylmethoxy]ethyl]-6-o-isopropoxyphenylthiopurine;
2-amino-9-[2-[(2,2,2-trifluoroethyl)phosphonylmethoxy]ethyl]-6-o-isobutoxyphenylthiopurine;
2-amino-9-[2-[(2,2,2-trifluoroethyl)phosphonylmethoxy]propyl]-6-phenylthiopurine;
2-amino-9-[2-[(2,2,2-trifluoroethyl)phosphonylmethoxy]propyl]-6-p-methoxyphenylthiopurine;
2-amino-9-[2-[(2,2,2-trifluoroethyl)phosphonylmethoxy]propyl]-6-m-methoxyphenylthiopurine;
2-amino-9-[2-[(2,2,2-trifluoroethyl)phosphonylmethoxy]propyl]-6-o-methoxyphenylthiopurine;
2-amino-9-[2-[(2,2,2-trifluoroethyl)phosphonylmethoxy]propyl]-6-p-ethoxyphenylthiopurine;
2-amino-9-[2-[(2,2,2-trifluoroethyl)phosphonylmethoxy]propyl]-6-m-ethoxyphenylthiopurine;
2-amino-9-[2-[(2,2,2-trifluoroethyl)phosphonylmethoxy]propyl]-6-o-ethoxyphenylthiopurine;
2-amino-9-[2-[(2,2,2-trifluoroethyl)phosphonylmethoxy]propyl]-6-p-butoxyphenylthiopurine;
2-amino-9-[2-[(2,2,2-trifluoroethyl)phosphonylmethoxy]propyl]-6-p-propoxyphenylthiopurine;
2-amino-9-[2-[(2,2,2-trifluoroethyl)phosphonylmethoxy]propyl]-6-p-isopropoxyphenylthiopurine;
2-amino-9-[2-[(2,2,2-trifluoroethyl)phosphonylmethoxy]propyl]-6-p-isobutoxyphenylthiopurine;
2-amino-9-[2-[(2,2,2-trifluoroethyl)phosphonylmethoxy]propyl]-6-m-butoxyphenylthiopurine;
2-amino-9-[2-[(2,2,2-trifluoroethyl)phosphonylmethoxy]propyl]-6-m-propoxyphenylthiopurine;
2-amino-9-[2-[(2,2,2-trifluoroethyl)phosphonylmethoxy]propyl]-6-m-isopropoxyphenylthiopurine;
2-amino-9-[2-[(2,2,2-trifluoroethyl)phosphonylmethoxy]propyl]-6-m-isobutoxyphenylthiopurine;
2-amino-9-[2-[(2,2,2-trifluoroethyl)phosphonylmethoxy]propyl]-6-o-butoxyphenylthiopurine;
2-amino-9-[2-[(2,2,2-trifluoroethyl)phosphonylmethoxy]propyl]-6-o-propoxyphenylthiopurine;
2-amino-9-[2-[(2,2,2-trifluoroethyl)phosphonylmethoxy]propyl]-6-o-isopropoxyphenylthiopurine;
2-amino-9-[2-[(2,2,2-trifluoroethyl)phosphonylmethoxy]propyl]-6-o-isobutoxyphenylthiopurine;
2-amino-9-[2-[(2,2,2-trifluoroethyl)phosphonylmethoxy]ethyl]-6-p-hydroxyphenylthiopurine;
2-amino-9-[2-[(2,2,2-trifluoroethyl)phosphonylmethoxy]ethyl]-6-m-hydroxyphenylthiopurine;
2-amino-9-[2-[(2,2,2-trifluoroethyl)phosphonylmethoxy]ethyl]-6-o-hydroxyphenylthiopurine;
2-amino-9-[2-[(2,2,2-trifluoroethyl)phosphonylmethoxy]propyl]-6-p-hydroxyphenylthiopurine;
2-amino-9-[2-[(2,2,2-trifluoroethyl)phosphonylmethoxy]propyl]-6-m-hydroxyphenylthiopurine;
2-amino-9-[2-[(2,2,2-trifluoroethyl)phosphonylmethoxy]propyl]-6-o-hydroxyphenylthiopurine;
2-amino-9-[2-(phosphonomethoxy)ethyl]-6-phenylthiopurine;
2-amino-9-[2-(phosphonomethoxy)ethyl]-6-p-methoxyphenylthiopurine;
2-amino-9-[2-(phosphonomethoxy)ethyl]-6-m-methoxyphenylthiopurine;
2-amino-9-[2-(phosphonomethoxy)ethyl]-6-o-methoxyphenylthiopurine;
2-amino-9-[2-(phosphonomethoxy)ethyl]-6-p-ethoxyphenylthiopurine;
2-amino-9-[2-(phosphonomethoxy)ethyl]-6-m-ethoxyphenylthiopurine;
2-amino-9-[2-(phosphonomethoxy)ethyl]-6-o-ethoxyphenylthiopurine;
2-amino-9-[2-(phosphonomethoxy)ethyl]-6-p-butoxyphenylthiopurine;
2-amino-9-[2-(phosphonomethoxy)ethyl]-6-p-propoxyphenylthiopurine;
2-amino-9-[2-(phosphonomethoxy)ethyl]-6-p-isopropoxyphenylthiopurine;
2-amino-9-[2-(phosphonomethoxy)ethyl]-6-p-isobutoxyphenylthiopurine;
2-amino-9-[2-(phosphonomethoxy)ethyl]-6-m-butoxyphenylthiopurine;
2-amino-9-[2-(phosphonomethoxy)ethyl]-6-m-propoxyphenylthiopurine;
2-amino-9-[2-(phosphonomethoxy)ethyl]-6-m-isopropoxyphenylthiopurine;
2-amino-9-[2-(phosphonomethoxy)ethyl]-6-m-isobutoxyphenylthiopurine;
2-amino-9-[2-(phosphonomethoxy)ethyl]-6-o-butoxyphenylthiopurine;
2-amino-9-[2-(phosphonomethoxy)ethyl]-6-o-propoxyphenylthiopurine;
2-amino-9-[2-(phosphonomethoxy)ethyl]-6-o-isopropoxyphenylthiopurine;
2-amino-9-[2-(phosphonomethoxy)ethyl]-6-o-isobutoxyphenylthiopurine;
2-amino-9-[2-(phosphonomethoxy)propyl]-6-phenylthiopurine;
2-amino-9-[2-(phosphonomethoxy)propyl]-6-p-methoxyphenylthiopurine;
2-amino-9-[2-(phosphonomethoxy)propyl]-6-m-methoxyphenylthiopurine;
2-amino-9-[2-(phosphonomethoxy)propyl]-6-o-methoxyphenylthiopurine;
2-amino-9-[2-(phosphonomethoxy)propyl]-6-p-ethoxyphenylthiopurine;
2-amino-9-[2-(phosphonomethoxy)propyl]-6-m-ethoxyphenylthiopurine;
2-amino-9-[2-(phosphonomethoxy)propyl]-6-o-ethoxyphenylthiopurine;
2-amino-9-[2-(phosphonomethoxy)propyl]-6-p-butoxyphenylthiopurine;
2-amino-9-[2-(phosphonomethoxy)propyl]-6-p-propoxyphenylthiopurine;
2-amino-9-[2-(phosphonomethoxy)propyl]-6-p-isopropoxyphenylthiopurine;
2-amino-9-[2-(phosphonomethoxy)propyl]-6-p-isobutoxyphenylthiopurine;
2-amino-9-[2-(phosphonomethoxy)propyl]-6-m-butoxyphenylthiopurine;
2-amino-9-[2-(phosphonomethoxy)propyl]-6-m-propoxyphenylthiopurine;
2-amino-9-[2-(phosphonomethoxy)propyl]-6-m-isopropoxyphenylthiopurine;
2-amino-9-[2-(phosphonomethoxy)propyl]-6-m-isobutoxyphenylthiopurine;
2-amino-9-[2-(phosphonomethoxy)propyl]-6-o-butoxyphenylthiopurine;
2-amino-9-[2-(phosphonomethoxy)propyl]-6-o-propoxyphenylthiopurine;
2-amino-9-[2-(phosphonomethoxy)propyl]-6-o-isopropoxyphenylthiopurine;
2-amino-9-[2-(phosphonomethoxy)propyl]-6-o-isobutoxyphenylthiopurine;
2-amino-9-[2-(phosphonomethoxy)ethyl]-6-p-hydroxyphenylthiopurine;
2-amino-9-[2-(phosphonomethoxy)ethyl]-6-m-hydroxyphenylthiopurine;
2-amino-9-[2-(phosphonomethoxy)ethyl]-6-o-hydroxyphenylthiopurine;
2-amino-9-[2-(phosphonomethoxy)propyl]-6-p-hydroxyphenylthiopurine;
2-amino-9-[2-(phosphonomethoxy)propyl]-6-m-hydroxyphenylthiopurine; and
2-amino-9-[2-(phosphonomethoxy)propyl]-6-o-hydroxyphenylthiopurine.

6. The method according to claim 1, wherein each of R² and R³ is a 2,2,2-trifluoroethyl group; and
R⁴ is a hydrogen atom or a methyl group.

7. The method according to claim 6, wherein the phosphonate nucleotide compound is selected from the group consisting of the following compounds:
2-amino-9-[2-[bis(2,2,2-trifluoroethyl)phosphonylmethoxy]ethyl]-6-phenylthiopurine;
2-amino-9-[2-[bis(2,2,2-trifluoroethyl)phosphonylmethoxy]ethyl]-6-p-methoxyphenylthiopurine;
2-amino-9-[2-[bis(2,2,2-trifluoroethyl)phosphonylmethoxy]ethyl]-6-m-methoxyphenylthiopurine;
2-amino-9-[2-[bis(2,2,2-trifluoroethyl)phosphonylmethoxy]ethyl]-6-o-methoxyphenylthiopurine;
2-amino-9-[2-[bis(2,2,2-trifluoroethyl)phosphonylmethoxy]ethyl]-6-p-ethoxyphenylthiopurine;
2-amino-9-[2-[bis(2,2,2-trifluoroethyl)phosphonylmethoxy]propyl]-6-phenylthiopurine;
2-amino-9-[2-[bis(2,2,2-trifluoroethyl)phosphonylmethoxy]propyl]-6-p-methoxyphenylthiopurine;
2-amino-9-[2-[bis(2,2,2-trifluoroethyl)phosphonylmethoxy]ethyl]-6-p-butoxyphenylthiopurine;
2-amino-9-[2-[bis(2,2,2-trifluoroethyl)phosphonylmethoxy]ethyl]-6-p-propoxyphenylthiopurine;
2-amino-9-[2-[bis(2,2,2-trifluoroethyl)phosphonylmethoxy]ethyl]-6-p-isopropoxyphenylthiopurine;
2-amino-9-[2-[bis(2,2,2-trifluoroethyl)phosphonylmethoxy]ethyl]-6-p-isobutoxyphenylthiopurine;
2-amino-9-[2-[bis(2,2,2-trifluoroethyl)phosphonylmethoxy]ethyl]-6-p-hydroxyphenylthiopurine;
2-amino-9-[2-[bis(2,2,2-trifluoroethyl)phosphonylmethoxy]ethyl]-6-m-hydroxyphenylthiopurine;
2-amino-9-[2-[bis(2,2,2-trifluoroethyl)phosphonylmethoxy]ethyl]-6-o-hydroxyphenylthiopurine;
2-amino-9-[2-[bis(2,2,2-trifluoroethyl)phosphonylmethoxy]propyl]-6-p-hydroxyphenylthiopurine;
2-amino-9-[2-[bis(2,2,2-trifluoroethyl)phosphonylmethoxy]propyl]-6-m-hydroxyphenylthiopurine;
2-amino-9-[2-[bis(2,2,2-trifluoroethyl)phosphonylmethoxy]propyl]-6-o-hydroxyphenylthiopurine;
2-amino-9-[2-[bis(2,2,2-trifluoroethyl)phosphonylmethoxy]ethyl]-6-m-ethoxyphenylthiopurine;
2-amino-9-[2-[bis(2,2,2-trifluoroethyl)phosphonylmethoxy]ethyl]-6-o-ethoxyphenylthiopurine;
2-amino-9-[2-[bis(2,2,2-trifluoroethyl)phosphonylmethoxy]ethyl]-6-m-butoxyphenylthiopurine;
2-amino-9-[2-[bis(2,2,2-trifluoroethyl)phosphonylmethoxy]ethyl]-6-m-propoxyphenylthiopurine;
2-amino-9-[2-[bis(2,2,2-trifluoroethyl)phosphonylmethoxy]ethyl]-6-m-isopropoxyphenylthiopurine;
2-amino-9-[2-[bis(2,2,2-trifluoroethyl)phosphonylmethoxy]ethyl]-6-m-isobutoxyphenylthiopurine;
2-amino-9-[2-[bis(2,2,2-trifluoroethyl)phosphonylmethoxy]ethyl]-6-o-butoxyphenylthiopurine;
2-amino-9-[2-[bis(2,2,2-trifluoroethyl)phosphonylmethoxy]ethyl]-6-o-propoxyphenylthiopurine;
2-amino-9-[2-[bis(2,2,2-trifluoroethyl)phosphonylmethoxy]ethyl]-6-o-isopropoxyphenylthiopurine;
2-amino-9-[2-[bis(2,2,2-trifluoroethyl)phosphonylmethoxy]ethyl]-6-o-isobutoxyphenylthiopurine;
2-amino-9-[2-[bis(2,2,2-trifluoroethyl)phosphonylmethoxy]propyl]-6-m-methoxyphenylthiopurine;
2-amino-9-[2-[bis(2,2,2-trifluoroethyl)phosphonylmethoxy]propyl]-6-o-methoxyphenylthiopurine;
2-amino-9-[2-[bis(2,2,2-trifluoroethyl)phosphonylmethoxy]propyl]-6-p-ethoxyphenylthiopurine;
2-amino-9-[2-[bis(2,2,2-trifluoroethyl)phosphonylmethoxy]propyl]-6-m-ethoxyphenylthiopurine;
2-amino-9-[2-[bis(2,2,2-trifluoroethyl)phosphonylmethoxy]propyl]-6-o-ethoxyphenylthiopurine;
2-amino-9-[2-[bis(2,2,2-trifluoroethyl)phosphonylmethoxy]propyl]-6-p-butoxyphenylthiopurine;
2-amino-9-[2-[bis(2,2,2-trifluoroethyl)phosphonylmethoxy]propyl]-6-p-propoxyphenylthiopurine;
2-amino-9-[2-[bis(2,2,2-trifluoroethyl)phosphonylmethoxy]propyl]-6-p-isopropoxyphenylthiopurine;
2-amino-9-[2-[bis(2,2,2-trifluoroethyl)phosphonylmethoxy]propyl]-6-p-isobutoxyphenylthiopurine;
2-amino-9-[2-[bis(2,2,2-trifluoroethyl)phosphonylmethoxy]propyl]-6-m-butoxyphenylthiopurine;
2-amino-9-[2-[bis(2,2,2-trifluoroethyl)phosphonylmethoxy]propyl]-6-m-propoxyphenylthiopurine;
2-amino-9-[2-[bis(2,2,2-trifluoroethyl)phosphonylmethoxy]propyl]-6-m-isopropoxyphenylthiopurine;
2-amino-9-[2-[bis(2,2,2-trifluoroethyl)phosphonylmethoxy]propyl]-6-m-isobutoxyphenylthiopurine;
2-amino-9-[2-[bis(2,2,2-trifluoroethyl)phosphonylmethoxy]propyl]-6-o-butoxyphenylthiopurine;
2-amino-9-[2-[bis(2,2,2-trifluoroethyl)phosphonylmethoxy]propyl]-6-o-propoxyphenylthiopurine;
2-amino-9-[2-[bis(2,2,2-trifluoroethyl)phosphonylmethoxy]propyl]-6-o-isopropoxyphenylthiopurine; and
2-amino-9-[2-[bis(2,2,2-trifluoroethyl)phosphonylmethoxy]propyl]-6-o-isobutoxyphenylthiopurine.

8. The method according to claim 1, wherein each of R² and R³ is a 2,2,2-trifluoroethyl group; and
R⁴ is a hydrogen atom.

9. The method according to claim 8, wherein the phosphonate nucleotide compound is selected from the group consisting of the following compounds:
2-amino-9-[2-[bis(2,2,2-trifluoroethyl)phosphonylmethoxy]ethyl]-6-phenylthiopurine;
2-amino-9-[2-[bis(2,2,2-trifluoroethyl)phosphonylmethoxy]ethyl]-6-p-methoxyphenylthiopurine;
2-amino-9-[2-[bis(2,2,2-trifluoroethyl)phosphonylmethoxy]ethyl]-6-m-methoxyphenylthiopurine;
2-amino-9-[2-[bis(2,2,2-trifluoroethyl)phosphonylmethoxy]ethyl]-6-o-methoxyphenylthiopurine;
2-amino-9-[2-[bis(2,2,2-trifluoroethyl)phosphonylmethoxy]ethyl]-6-p-ethoxyphenylthiopurine;
2-amino-9-[2-[bis(2,2,2-trifluoroethyl)phosphonylmethoxy]ethyl]-6-p-butoxyphenylthiopurine;
2-amino-9-[2-[bis(2,2,2-trifluoroethyl)phosphonylmethoxy]ethyl]-6-p-propoxyphenylthiopurine;
2-amino-9-[2-[bis(2,2,2-trifluoroethyl)phosphonylmethoxy]ethyl]-6-p-isopropoxyphenylthiopurine;
2-amino-9-[2-[bis(2,2,2-trifluoroethyl)phosphonylmethoxy]ethyl]-6-p-isobutoxyphenylthiopurine;
2-amino-9-[2-[bis(2,2,2-trifluoroethyl)phosphonylmethoxy]ethyl]-6-p-hydroxyphenylthiopurine;
2-amino-9-[2-[bis(2,2,2-trifluoroethyl)phosphonylmethoxy]ethyl]-6-m-hydroxyphenylthiopurine;
2-amino-9-[2-[bis(2,2,2-trifluoroethyl)phosphonylmethoxy]ethyl]-6-o-hydroxyphenylthiopurine;
2-amino-9-[2-[bis(2,2,2-trifluoroethyl)phosphonylmethoxy]ethyl]-6-m-ethoxyphenylthiopurine;
2-amino-9-[2-[bis(2,2,2-trifluoroethyl)phosphonylmethoxy]ethyl]-6-o-ethoxyphenylthiopurine;
2-amino-9-[2-[bis(2,2,2-trifluoroethyl)phosphonylmethoxy]ethyl]-6-m-butoxyphenylthiopurine;
2-amino-9-[2-[bis(2,2,2-trifluoroethyl)phosphonylmethoxy]ethyl]-6-m-propoxyphenylthiopurine;
2-amino-9-[2-[bis(2,2,2-trifluoroethyl)phosphonylmethoxy]ethyl]-6-m-isopropoxyphenylthiopurine;
2-amino-9-[2-[bis(2,2,2-trifluoroethyl)phosphonylmethoxy]ethyl]-6-m-isobutoxyphenylthiopurine;
2-amino-9-[2-[bis(2,2,2-trifluoroethyl)phosphonylmethoxy]ethyl]-6-o-butoxyphenylthiopurine;
2-amino-9-[2-[bis(2,2,2-trifluoroethyl)phosphonylmethoxy]ethyl]-6-o-propoxyphenylthiopurine;
2-amino-9-[2-[bis(2,2,2-trifluoroethyl)phosphonylmethoxy]ethyl]-6-o-isopropoxyphenylthiopurine; and
2-amino-9-[2-[bis(2,2,2-trifluoroethyl)phosphonylmethoxy]ethyl]-6-o-isobutoxyphenylthiopurine.

10. The method according to claim 1, wherein R¹ is a hydrogen atom, a hydroxyl group or a C₁-C₂ alkoxy group;
each of R² and R³ is a 2,2,2-trifluoroethyl group; and
R⁴ is a hydrogen atom.

11. The method according to claim 10, wherein the phosphonate nucleotide compound is selected from the group consisting of the following compounds:
2-amino-9-[2-[bis(2,2,2-trifluoroethyl)phosphonylmethoxy]ethyl]-6-phenylthiopurine;
2-amino-9-[2-[bis(2,2,2-trifluoroethyl)phosphonylmethoxy]ethyl]-6-p-methoxyphenylthiopurine;
2-amino-9-[2-[bis(2,2,2-trifluoroethyl)phosphonylmethoxy]ethyl]-6-m-methoxyphenylthiopurine;
2-amino-9-[2-[bis(2,2,2-trifluoroethyl)phosphonylmethoxy]ethyl]-6-o-methoxyphenylthiopurine;
2-amino-9-[2-[bis(2,2,2-trifluoroethyl)phosphonylmethoxy]ethyl]-6-p-hydroxyphenylthiopurine;
2-amino-9-[2-[bis(2,2,2-trifluoroethyl)phosphonylmethoxy]ethyl]-6-m-hydroxyphenylthiopurine;
2-amino-9-[2-[bis(2,2,2-trifluoroethyl)phosphonylmethoxy]ethyl]-6-o-hydroxyphenylthiopurine; and
2-amino-9-[2-[bis(2,2,2-trifluoroethyl)phosphonylmethoxy]ethyl]-6-p-ethoxyphenylthiopurine.

12. The method according to claim 1, wherein the drug resistant virus is a human immunodeficiency virus or hepatitis B virus.

13. The method according to claim 1, wherein the drug resistant virus is a hepatitis B virus.
